SEQUENCE LISTING (46 sequences)

SEQ ID NO: 1 - protein, 10 aa, Artificial Sequence
  a peptide derived from ORF1ab polyprotein of SARS-CoV-2
SLFDMSKFPL

SEQ ID NO: 2 - protein, 10 aa, Artificial Sequence
  a peptide derived from ORF1ab polyprotein of SARS-CoV-2
LMYKGLPWNV

SEQ ID NO: 3 - protein, 10 aa, Artificial Sequence
  a peptide derived from ORF1ab polyprotein of SARS-CoV-2
FVLWAHGFEL

SEQ ID NO: 4 - protein, 10 aa, Artificial Sequence
  a peptide derived from ORF1ab polyprotein of SARS-CoV-2
YMPASWVMRI

SEQ ID NO: 5 - protein, 10 aa, Artificial Sequence
  a peptide derived from ORF1ab polyprotein of SARS-CoV-2
MMGFKMNYQV

SEQ ID NO: 6 - protein, 10 aa, Artificial Sequence
  a peptide derived from ORF1ab polyprotein of SARS-CoV-2
FELTSMKYFV

SEQ ID NO: 7 - protein, 10 aa, Artificial Sequence
  a peptide derived from ORF1ab polyprotein of SARS-CoV-2
FTYASALWEI

SEQ ID NO: 8 - protein, 9 aa, Artificial Sequence
  a peptide derived from ORF1ab polyprotein of SARS-CoV-2
MMILSDDAV

SEQ ID NO: 9 - protein, 10 aa, Artificial Sequence
  a peptide derived from N polyprotein of SARS-CoV-2
FGMSRIGMEV

SEQ ID NO: 10 - protein, 10 aa, Artificial Sequence
  a peptide derived from ORF1ab polyprotein of SARS-CoV-2
TLMIERFVSL

SEQ ID NO: 11 - protein, 10 aa, Artificial Sequence
  a peptide derived from ORF1ab polyprotein of SARS-CoV-2
YVYNPFMIDV

SEQ ID NO: 12 - protein, 10 aa, Artificial Sequence
  a peptide derived from ORF1ab polyprotein of SARS-CoV-2
NVLAWLYAAV

SEQ ID NO: 13 - protein, 10 aa, Artificial Sequence
  a peptide derived from ORF1ab polyprotein of SARS-CoV-2
TLEPEYFNSV

SEQ ID NO: 14 - protein, 10 aa, Artificial Sequence
  a peptide derived from ORF1ab polyprotein of SARS-CoV-2
YSFLPGVYSV

SEQ ID NO: 15 - protein, 10 aa, Artificial Sequence
  a peptide derived from ORF1ab polyprotein of SARS-CoV-2
FMIDVQQWGF

SEQ ID NO: 16 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 17 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 19 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 20 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 21 - protein, 7096 aa, Severe acute respiratory syndrome coronavirus 2 [sequence omitted]

SEQ ID NO: 22 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 23 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 24 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 25 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 26 - protein, 38 aa, Severe acute respiratory syndrome coronavirus 2
MGYINVFAFPFTIYSLLLCRMNSRNYIAQVDVVNFNLT

SEQ ID NO: 27 - protein, 4 aa, Artificial Sequence
  a linker peptide
NKRK

SEQ ID NO: 28 - DNA, 22 bp, Artificial Sequence
  a PCR primer for the TCR analysis
gtctaccaggcattcgcttcat

SEQ ID NO: 29 - DNA, 24 bp, Artificial Sequence
  a PCR primer for the TCR analysis
tcagctggaccacagccgcagcgt

SEQ ID NO: 30 - DNA, 21 bp, Artificial Sequence
  a PCR primer for the TCR analysis
tcagaaatcctttctcttgac

SEQ ID NO: 31 - DNA, 24 bp, Artificial Sequence
  a PCR primer for the TCR analysis
ctagcctctggaatcctttctctt

SEQ ID NO: 32 - protein, 15 aa, Homo sapiens
CAVRRTYFGNEKLTF

SEQ ID NO: 33 - protein, 13 aa, Homo sapiens
CSARGSDSYEQYF

SEQ ID NO: 34 - protein, 11 aa, Homo sapiens
CAITGANNLFF

SEQ ID NO: 35 - protein, 16 aa, Homo sapiens
CSAEDRGSNSNQPQHF

SEQ ID NO: 36 - protein, 14 aa, Homo sapiens
CAVRDPRNDYKLSF

SEQ ID NO: 37 - protein, 11 aa, Homo sapiens
CATQVNTEAFF

SEQ ID NO: 38 - protein, 12 aa, Homo sapiens
CAFLSGNTPLVF

SEQ ID NO: 39 - protein, 13 aa, Homo sapiens
CASSPAAYSPLHF

SEQ ID NO: 40 - DNA, 19 bp, Artificial Sequence
  a PCR primer for the TCR analysis
tgtaaaacgacggccagtg

SEQ ID NO: 41 - DNA, 16 bp, Artificial Sequence
  a PCR primer for the TCR analysis
ctggccgtcgttttac

SEQ ID NO: 42 - DNA, 20 bp, Artificial Sequence
  a PCR primer for the TCR analysis
caggaaacagctatgaccat

SEQ ID NO: 43 - DNA, 17 bp, Artificial Sequence
  a PCR primer for the TCR analysis
caggaaacagctatgac

SEQ ID NO: 44 - DNA, 54 bp, Artificial Sequence
  a PCR primer for the TCR analysis
gtctcgtgggctcggagatgtgtataagagacagtatcaacgcagagtggccat

SEQ ID NO: 45 - DNA, 58 bp, Artificial Sequence
  a PCR primer for the TCR analysis
tcgtcggcagcgtcagatgtgtataagagacagdvhdvtctgatggctcaaacacagc

SEQ ID NO: 46 - DNA, 58 bp, Artificial Sequence
  a PCR primer for the TCR analysis
tcgtcggcagcgtcagatgtgtataagagacagdbdhhcagggtcagggttctggata

(19) United States
(12) Patent Application Publication
(10) Pub. No.: US 2024/0067679 A1
(43) Pub. Date: Feb. 29, 2024

(54) SARS-COV-2 PROTEIN-DERIVED PEPTIDE AND VACCINE CONTAINING SAME

(71) Applicants: Onco Therapy Science, Inc., Kawasaki-shi, Kanagawa (JP); Cancer Precision Medicine, Inc., Kawasaki-shi, Kanagawa (JP)

(72) Inventors: KAZUMA KIYOTANI, Kawasaki-shi, Kanagawa (JP); YUSUKE NAKAMURA, Kawasaki-shi, Kanagawa (JP); TETSURO HIKICHI, Kawasaki-shi, Kanagawa (JP)

(73) Assignees: Onco Therapy Science, Inc., Kawasaki-shi, Kanagawa (JP); Cancer Precision Medicine, Inc., Kawasaki-shi, Kanagawa (JP)

(21) Appl. No.: 18/270,590

(22) PCT Filed: Dec. 28, 2021
(86) PCT No.: PCT/JP2021/048846

§ 371 Date: Jun. 30, 2023

Related U.S. Application Data
(60) Provisional application No. 63/236,927, filed on Aug. 25, 2021.

(30) Foreign Application Priority Data
Jan. 5, 2021 (JP) 2021-000382
Apr. 30, 2021 (WO) PCT/JP2021/017162

(51) Int. Cl. C07K 14/005; A61K 39/215; C07K 14/725; C12Q 1/6881

(52) U.S. Cl. CPC C07K 14/005; A61K 39/215; C07K 14/7051; C12Q 1/6881; A61K 2039/605; C12N 2770/20022; C12N 2770/20034

(57) ABSTRACT

The present invention provides epitope peptides which are derived from SARS-CoV-2 proteins and have the ability to induce cytotoxic T cells. The present invention also provides polynucleotides encoding the peptides, antigen-presenting cells that present the peptides, and cytotoxic T cells that target the peptides, and methods of inducing the antigen-presenting cells or CTLs. The present invention further provides compositions and pharmaceutical compositions containing them as active ingredients. Moreover, the present invention provides methods of treating and/or preventing coronavirus infectious diseases, and/or methods of suppressing the aggravation of coronavirus infectious diseases by using the peptides, polynucleotides, antigen-presenting cells, cytotoxic T cells, or pharmaceutical compositions of the present invention. The present invention also provides methods of inducing an immune response against coronavirus infection. Furthermore, the present invention provides methods of examining the history of coronavirus infection by detecting TCR sequences of a subject.

The present application claims the benefit of Japanese Patent Application No. 2021-382 filed on Jan. 5, 2021, International Application No. PCT/JP2021/017162 filed on Apr. 30, 2021 and the U.S. Provisional Application No. 63/236,927 filed on Aug. 25, 2021, the entire contents of which are incorporated by reference herein.

### TECHNICAL FIELD

The present invention relates to the field of biological science, more specifically to the field of prevention of virus infectious diseases. In particular, the present invention relates to novel peptides that are effective as vaccines for preventing infection, methods for either or both of preventing and treating infectious diseases using the peptide(s), and pharmaceutical compositions comprising the peptide(s)

### BACKGROUND ART

The respiratory infectious disease (Coronavirus Disease 2019: COVID-19) caused by infection with the novel coronavirus (Severe Acute Respiratory Syndrome Coronavirus 2: SARS-CoV-2) has spread around the world since it was first reported in Wuhan, China.

According to the report from the World Health Organization (WHO), as of December 2020, the number of infected people exceeded 70 million and the number of deaths reached 1.59 million. Moreover, as of April 2021, the number of infected people exceeded 120 million and the number of deaths reached 2.8 million. Effective means for stopping the spread of infection include preventive vaccination; and vaccines against COVID-19 are being put to practical use.

It has been previously known that BCG vaccine has a preventive effect even against infectious diseases other than tuberculosis. A recent study has also reported that BCG vaccination of elderly people aged 65 years and older showed a suppressive effect on incidence of infectious diseases (NPL 1: Giamarellos-Bourboulis E J et al., Cell 2020, 183(2): 315-323. e9).

In addition, an association has been suggested between suppression of fatality rates or growth rates of deaths in COVID-19 and BCG vaccination (NPL 2: Toyoshima Y et al., J Hum Genet 2020, 65(12): 1075-1082; NPL 3: Berg MK et al., Science Advances 2020, 6(32): eabc1463).

In the human defense mechanism against viral infection, innate immune responses led by immune cells (such as macrophages, NK cells, and neutrophils) work first, and then antigen-specific immune responses are developed by acquired immunity (humoral immunity and cellular immunity) led by B cells and T cells. After BCG vaccination, the activity of transcribing inflammatory cytokines is enhanced in immune cells, and infection with a virus in this state (trained immunity) triggers rapid release of inflammatory cytokines in the body. This is presumed to result in humoral immune responses by activated B cells and cellular immune responses by T cells suppressing viral proliferation (NPL 4: Netea MG et al., Cell 2020, 181(5): 969-977).

It is the spike protein that plays a central function in the entry of SARS-CoV-2 into human cells (NPL 5: Hoffmann M et al., Cell 2020, 181(2): 271-280). Most of the vaccines against COVID-19 currently under research and development, including the mRNA vaccine “BNT162b2” approved in England and United States, are primarily aimed at inducing neutralizing antibodies (inducing humoral immunity) against the spike protein of SARS-CoV-2 (NPL 6: Jeyanathan M et al., Nat Rev Immunol 2020, 20(10): 615-632). However, repeated proliferation of SARS-CoV-2 may cause mutations in its viral genome. When a mutation occurs in the gene encoding the spike protein, vaccine-induced neutralizing antibodies may become ineffective. Moreover, studies conducted on people who had recovered from COVID-19 suggest that neutralizing antibodies induced in the human body may be short-lived (NPL 7: Ibarrondo F J et al., N Engl J Med 2020, 383(11): 1085-1087; NPL 8: Long QX et al., Nat Med 2020, 26(8): 1200-1204). Accordingly, problems remain in the development of vaccines aimed at inducing humoral immunity by neutralizing antibodies.

Cellular immunity by T cells is also very important as a defense reaction against infectious diseases. Cytotoxic T lymphocytes (CTLs) are CD8-positive T cells and are induced by presentation of viral antigens (epitope peptides derived from viral proteins) by dendritic cells (DCs). Then, CTLs recognize epitope peptides presented on human leukocyte antigen (HLA) class I molecules expressed on the surface of virus-infected cells and kill these cells. This action destroys the infected cells, where viral particles are replicated, and results in suppression of viral proliferation. Compared to neutralizing antibodies, which mainly inhibit new infection of cells with replicated viral particles, CTLs, which target the replication process itself of viral particles, have a more direct effect in preventing viral proliferation. Accordingly, CTLs are considered to play a role in eliminating viruses from the living body and suppressing aggravation of infectious diseases. The report that the peripheral blood of patients with severe COVID-19 shows a marked decrease in CD8-positive T cells compared to healthy individuals suggests that CTLs are an important factor in suppressing aggravation of COVID-19 (NPL 9: Zheng M et al., Cell Mol Immunol 2020, 17(5): 533-535).

Furthermore, part of CTLs are known to remain in the body as memory T cells for a long time. In 2003, the SARS-CoV epidemic occurred and more than 8,000 people were infected. Because of the fact that, 17 years after the epidemic, CTLs against SARS-CoV were still detected in the blood of people who had recovered (NPL 10: Le Bert N et al., Nature 2020, 584(7821): 457-462), CTLs against SARS-CoV-2 may also remain in the body for a long time after being induced.

Accordingly, a vaccine aimed at eliciting cellular immunity can be an effective preventive measure against COVID-19. It is considered that CTLs induced by a vaccine comprising an epitope peptide derived from a SARS-CoV-2 protein remain in the body as memory T cells and show cytotoxic activity against virus-infected cells rapidly after SARS-CoV-2 infection, leading to suppression of aggravation of COVID-19. Thus, it is desired to identify an epitope peptide derived from a SARS-CoV-2 protein that can induce CTLs in the human body.

Candidate epitope peptides determined based on bioinformatics have been reported; however, it has not been verified whether or not they actually bind to an HLA and have the ability to induce CTLs, and no epitope peptide has been identified (NPL 11: Grifoni A et al., Cell Host Microbe 2020, 27(4): 671-680; NPL12: Crooke SN et al., Sci Rep 2020, 10(1): 14179).

Furthermore, in taking measures to stop the spread of SARS-CoV-2 infection, it is important to accurately understand each individual's history of infection. Currently, it has become mainstream to perform an antibody test in order to examine the history of infection; however, antibody titers may be very low in asymptomatic people and infected people showing only mild symptoms (see NPL 13: Marchi S et al., PLoS One 2021, 16(7): e0253977; NPL 8: Long Q X et al., Nat Med 2020, 26(8): 1200-1204). Such facts may limit antibody tests, and a new testing method is desired. In addition to humoral immunity, led by antibodies, cellular immunity, led by T cells, also plays an important role in controlling viral infection. Cytotoxic T lymphocytes (CTLs) recognize, through their T cell receptors (TCRs), viral protein-derived peptides presented on major histocompatibility complex (MHC) class I molecules on the surface of virus-infected cells, and then injure these cells. The T cell response to viral infection is considered to be manifested as, e.g., a marked increase or decrease in a particular TCR in comprehensive analysis of TCRs derived from peripheral blood T cells. TCRs derived from SARS-CoV-2-specific CTLs may serve as important markers for understanding SARS-CoV-2 infection.

### CITATION LIST

### Non-Patent Literature

[NPL 1] Giamarellos-Bourboulis E J et al., Cell 2020, 183(2): 315-323. e9

[NPL 2] Toyoshima Y et al., J Hum Genet 2020, 65(12): 1075-1082

[NPL 3] Berg MK et al., Science Advances 2020, 6(32): eabc1463

[NPL 4] Netea M G et al., Cell 2020, 181(5): 969-977

[NPL 5] Hoffmann M et al., Cell 2020, 181(2): 271-280

[NPL 6] Jeyanathan M et al., Nat Rev Immunol 2020, 20(10): 615-632

[NPL 7] Ibarrondo F J et al., N Engl J Med 2020, 383(11): 1085-1087

[NPL 8] Long Q X et al., Nat Med 2020, 26(8): 1200-1204

[NPL 9] Zheng M et al., Cell Mol Immunol 2020, 17(5): 533-535

[NPL 10] Le Bert N et al., Nature 2020, 584(7821): 457-462

[NPL 11] Grifoni A et al., Cell Host Microbe 2020, 27(4): 671-680

[NPL 12] Crooke S N et al., Sci Rep 2020, 10(1): 14179

[NPL 13] Marchi S et al., PLoS One 2021, 16(7): e0253977

### SUMMARY OF INVENTION

The present invention provides epitope peptides that bind to HLA-A*02:01 and exhibit the ability to induce CTLs, which are selected from peptides derived from four structural proteins and six non-structural proteins that SARS-CoV-2 (reference sequence: GenBank accession number MN908947 (SEQ ID NO: 16)) has. The present invention also provides epitope peptides that bind to both HLA-A*02:01 and HLA-A*02:06 and exhibit the ability to induce CTLs. The present invention further provides epitope peptides that bind to both HLA-A*02:01 and HLA-A*24:02 and exhibit the ability to induce CTLs. The present invention also provides epitope peptides that binds to all HLA-A*02:01, HLA-A*02:06 and HLA-A*24:02 and exhibit the ability to induce CTLs.

The four structural proteins specifically refer to the following proteins: spike protein

(reference sequence: GenBank accession number QHD43416 (SEQ ID NO: 17)); envelope protein

(reference sequence: GenBank accession number QHD43418 (SEQ ID NO: 18)); matrix protein

(reference sequence: GenBank accession number QHD43419 (SEQ ID NO: 19)); and nucleoprotein

(reference sequence: GenBank accession number QHD43423 (SEQ ID NO: 20)).

On the other hand, the six non-structural proteins specifically refer to the following proteins:

### ORF1ab

(reference sequence: GenBank accession number QHD43415 (SEQ ID NO: 21));

### ORF3a

(reference sequence: GenBank accession number QHD43417 (SEQ ID NO: 22));

### ORF6

(reference sequence: GenBank accession number QHD43420 (SEQ ID NO: 23));

### ORF7a

(reference sequence: GenBank accession number QHD43421 (SEQ ID NO: 24));

### ORF8

(reference sequence: GenBank accession number QHD43422 (SEQ ID NO: 25)); and

### ORF10

(reference sequence: GenBank accession number QHI42199 (SEQ ID NO: 26)).

The epitope peptides of the present invention are demonstrated to be able to induce specific and potent immune responses against COVID-19 targeting HLA-A*24:02-positive individuals, who constitute a large population in Asian countries including Japan, or targeting HLA-A*02:01-positive individuals and HLA-A*02:06-positive individuals, who constitute a large population in Europe and the United States (Cao K et al., Hum Immunol 2001, 62(9): 1009-1030; Gonzalez-Galarza F F et al., Nucleic Acids Res 2020, 48(D1): D783-D788). Some of the epitope peptides of the present invention are demonstrated to be able to induce a specific and potent immune response against COVID-19 also targeting HLA-A*24:02-positive individuals, who constitute a large population in Asian countries including Japan.

Since the amino acid sequences of the epitope peptides provided in the present invention have low similarity to those derived from human proteins, creation of highly safe vaccines that are less likely to cause unexpected side reactions can be expected. On the other hand, since those amino acid sequences are commonly found also in SARS-CoV and MERS-CoV, which were epidemic in the past, they may also be found similarly in proteins of new types of coronaviruses that will emerge in the future. Accordingly, vaccines comprising such epitope peptide can be effective against not only the current SARS-CoV-2 but also coronavirus infectious diseases that will be prevalent in the future.

The present invention also provides compositions comprising one or more types of peptides of the present invention, one or more types of polynucleotides encoding one or more types of peptides of the present invention, APCs of the present invention, exosomes presenting peptides of the present invention, and/or CTLs of the present invention. The compositions of the present invention are preferably pharmaceutical compositions. The pharmaceutical compositions of the present invention can be used for treating and/or preventing coronavirus infectious diseases, as well as suppressing aggravation. They can also be used for inducing an immune response against coronavirus infection. When administered to a subject, a peptide of the present invention is presented on the surface of an APC, and as a result CTLs targeting the peptide are induced. Therefore, another objective of the present invention is to provide compositions inducing CTLs, wherein the compositions comprise one or more types of peptides of the present invention, one or more types of polynucleotides encoding one or more types of peptides of the present invention, APCs of the present invention, and/or exosomes presenting peptides of the present invention.

A further objective of the present invention is to provide methods of inducing APCs having CTL-inducing ability, wherein the methods comprise a step of contacting one or more types of peptides of the present invention with an APC, or a step of introducing a polynucleotide encoding any one peptide of the present invention into an APC.

The present invention further provides a method of inducing CTLs, comprising a step of co-culturing a CD8-positive T cell with an APC that presents on its surface a complex of an HLA antigen and a peptide of the present invention, a step of co-culturing a CD8-positive T cell with an exosome that presents on its surface a complex of an HLA antigen and a peptide of the present invention, or a step of introducing into a CD8-positive T cell a vector comprising a polynucleotide encoding each subunit of a T cell receptor (TCR) capable of binding to a peptide of the present invention presented by an HLA antigen on a cell surface.

A further objective of the present invention is to provide isolated APCs that present on their surface a complex of an HLA antigen and a peptide of the present invention. The present invention further provides isolated CTLs targeting a peptide of the present invention. These APCs and CTLs can be used in immunotherapy for coronavirus infectious diseases.

Another objective of the present invention is to provide methods of inducing an immune response against coronavirus infection in a subject, wherein the methods comprise a step of administering to the subject a peptide(s) of the present invention, a polynucleotide(s) encoding the peptide(s), an APC(s) of the present invention, an exosome(s) presenting a peptide(s) of the present invention, and/or a CTL(s) of the present invention. Another objective of the present invention is to provide methods of treating and/or preventing coronavirus infectious disease, as well as suppressing aggravation in a subject, wherein the methods comprise a step of administering to the subject a peptide(s) of the present invention, a polynucleotide(s) encoding the peptide(s), an APC(s) of the present invention, an exosome(s) presenting a peptide(s) of the present invention, and/or a CTL(s) of the present invention.

The present invention also relates to TCRs expressed by T cells that recognize a SARS-CoV-2 protein-derived peptide. TCR is a protein molecule consisting of a dimer of an α chain and a β chain. Human T cells recognize peptides presented on MHC class I (also known as human leukocyte antigen, HLA) molecules through TCRs. As a result, T cell proliferation, differentiation, cytokine production, cytotoxic substance (perforin and granzymes) secretion and the like are induced.

The TCRα gene comprises Vα, Jα, and Cα genes. The TCRβ gene comprises Vβ, Dβ, Jβ, and Cβ genes. The regions determining the specificity of TCR for a peptide are called complementarity determining regions (CDRs), which include CDR1, CDR2, and CDR3. Of these, CDR3 contacts a peptide directly, and therefore its amino acid sequence is very important in determining specificity. In the TCR a chain, V-J corresponds to CDR3, and in the TCR β chain, V-D and D-J correspond to CDR3. Insertion and deletion of nucleotides generate their diversity.

SARS-CoV-2 infection or vaccination may selectively increase T cells that recognize SARS-CoV-2 protein-derived peptides. For the purpose of examining such an immune response in the form of an altered frequency of detection of a particular TCR in the peripheral blood, the amino acid sequences of TCRs shown herein (in particular, CDR3 amino acid sequences), which have been identified from T cells recognizing SARS-CoV-2 protein-derived peptides, are of great use. That is, an objective of the present invention is to provide methods of knowing the immune response of a subject to SARS-CoV-2, the method comprising determining the detection frequency of TCRs in the peripheral blood of the subject.

In addition to the above, other objects and features of the present invention will become more fully apparent when the following detailed description is read in conjunction with the accompanying figures and examples. However, it is to be understood that both the foregoing summary of the present invention and the following detailed description are of exemplified embodiments, and not restrictive of the present invention or other alternate embodiments of the present invention. In particular, while the present invention is described herein with reference to a number of specific embodiments, it will be appreciated that the description is illustrative of the present invention and is not constructed as limiting of the present invention. Various modifications and applications may occur to those who are skilled in the art, without departing from the spirit and the scope of the present invention, as described by the appended claims.

Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

FIG. 1 is composed of images showing the results of IFN-γ enzyme-linked immunospot (ELISPOT) assays performed using cells induced with peptides derived from SARS-CoV-2 proteins. In the figure, “(+)” indicates IFN-γ production for HLA-A*02:01-expressing target cells (T2 cells) pulsed with a peptide of interest, and “(−)” indicates IFN-γ production for T2 cells not pulsed with any peptide (negative control). Comparison with the negative control shows that peptide-specific IFN-γ production was observed for Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15) (FIG. 1a). On the other hand, Peptide 8 (SEQ ID NO: 8) is shown as an example of typical negative data for which no peptide-specific IFN-γ production was observed (FIG. 1b).

FIG. 2 is composed of line graphs showing the results of measuring IFN-γ produced by cells stimulated with Peptide 2 (SEQ ID NO: 2), Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15), by an enzyme-linked immunosorbent assay (ELISA). These results show that after induction with the peptides, HLA-A*02:01-restricted CTL lines were established that produced IFN-γ in a peptide-specific manner. In the figure, “(+)” indicates IFN-γ production of a CTL line for HLA-A*02:01-expressing target cells (T2 cells) pulsed with a peptide of interest, and “(−)” indicates IFN-γ production of a CTL line for T2 cells not pulsed with any peptide. The R/S ratio represents the ratio of the number of CTL line cells, which are responder cells (Responder cells), to the number of target cells (Stimulator cells) that stimulate them.

FIG. 3 is composed of images showing the results of IFN-γ enzyme-linked immunospot (ELISPOT) assays performed using cells induced with peptides derived from SARS-CoV-2 proteins. In the figure, “(+)” indicates IFN-γ production for HLA-A*24:02-expressing target cells (TISI cells) pulsed with a peptide of interest, and “(−)” indicates IFN-γ production for TISI cells not pulsed with any peptide (negative control). Comparison with the negative control shows that peptide-specific IFN-γ production was observed for Peptide 3 (SEQ ID NO: 3), and Peptide 11 (SEQ ID NO: 11) (FIG. 3a). On the other hand, Peptide 4 (SEQ ID NO: 4) is shown as an example of typical negative data for which no peptide-specific IFN-γ production was observed (FIG. 3b).

FIG. 4 is composed of line graphs showing the results of measuring IFN-γ produced by cells stimulated with Peptide 3 (SEQ ID NO: 3) or Peptide 11 (SEQ ID NO: 11), by an enzyme-linked immunosorbent assay (ELISA). These results show that after induction with the peptides, HLA-A*24:02-restricted CTL lines were established that produced IFN-γ in a peptide-specific manner. In the figure, “(+)” indicates IFN-γ production of a CTL line for HLA-A*24:02-expressing target cells (TISI cells) pulsed with a peptide of interest, and “(−)” indicates IFN-γ production of a CTL line for TISI cells not pulsed with any peptide. The R/S ratio represents the ratio of the number of CTL line cells, which are responder cells (Responder cells), to the number of target cells (Stimulator cells) that stimulate them.

FIG. 5 is composed of images showing the results of IFN-γ enzyme-linked immunospot (ELISPOT) assays performed using cells induced with peptides derived from SARS-CoV-2 proteins. In the figure, “(+)” indicates IFN-γ production for HLA-A*02:06-expressing target cells (HEV0011 cells) pulsed with a peptide of interest, and “(−)” indicates IFN-γ production for HEV0011 cells not pulsed with any peptide (negative control). Comparison with the negative control shows that peptide-specific IFN-γ production was observed for Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) (FIG. 5a). On the other hand, Peptide 2 (SEQ ID NO: 2) is shown as an example of typical negative data for which no peptide-specific IFN-γ production was observed (FIG. 5b).

FIG. 6 is composed of line graphs showing the results of measuring IFN-γ produced by cells stimulated with Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15), by an enzyme-linked immunosorbent assay (ELISA). These results show that after induction with the peptides, HLA-A*02:06-restricted CTL lines were established that produced IFN-γ in a peptide-specific manner. In the figure, “(+)” indicates IFN-γ production of a CTL line for HLA-A*02:06-expressing target cells (HEV0011 cells) pulsed with a peptide of interest, and “(−)” indicates IFN-γ production of a CTL line for HEV0011 cells not pulsed with any peptide. The R/S ratio represents the ratio of the number of CTL line cells, which are responder cells (Responder cells), to the number of target cells (Stimulator cells) that stimulate them.

FIG. 7 is composed of line graphs showing IFN-γ production of CTL clones specific to SARS-CoV-2 protein-derived peptides that were established by limiting dilution method from PBMCs after in vitro CTL induction. IFN-γ production by CTL clones was observed for target cells (+) pulsed with SARS-CoV-2 protein-derived peptides, while no significant IFN-γ production by CTL clones was observed for target cells (−) not pulsed with a peptide. Thus, it was confirmed that the CTL clones recognized the SARS-CoV-2 protein-derived peptides presented on HLAs. The R/S ratio represents the ratio of the number of CTL clone cells, which are responder cells (Responder cells), to the number of target cells (Stimulator cells) that stimulate them.

FIG. 8 is composed of the results of tetramer assays performed on PBMCs collected from individuals not infected with SARS-CoV-2. Populations of tetramer-positive CD8-positive T cells that recognize SARS-CoV-2 protein-derived peptides presented on HLA-A*02:01 were detected in the PBMCs derived from individuals not infected with SARS-CoV-2.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

### I. Definitions

The words “a”, “an”, and “the” as used herein mean “at least one” unless otherwise specifically indicated.

The terms “isolated” and “purified” used in relation with a substance (for example, peptide, antibody, polynucleotide or such) indicate that the substance does not substantially contain at least one substance that may else be included in a natural source. Thus, an isolated or purified peptide refers to a peptide that does not substantially contain another cellular material, for example, carbohydrate, lipid and other contaminating proteins from the cell or tissue source from which the peptide is derived. When the peptide is chemically synthesized, an isolated or purified peptide refers to a peptide that does not substantially contain a precursor substance or another chemical substance. The phrase “does not substantially contain a cellular material” includes peptide preparations in which the peptide is separated from cellular components of the cells from which it is isolated or recombinantly produced. Thus, a peptide that does not substantially contain a cellular material encompasses peptide preparations that contain less than about 30%, 20%, 10%, or 5%, 3%, 2% or 1% (dry weight basis) of other cellular materials. When the peptide is recombinantly produced, an isolated or purified peptide does not substantially contain culture medium, which encompasses peptide preparations that contain culture medium less than about 20%, 10%, or 5%, 3%, 2% or 1% (dry weight basis) of the volume of the peptide preparation. When the peptide is chemically synthesized, an isolated or purified peptide does not substantially contain a precursor substance or other chemical substances, which encompasses peptide preparations that contain a precursor substance or other chemical substances less than about 30%, 20%, 10%, 5%, 3%, 2% or 1% (dry weight basis) of the volume of the peptide preparation. That a particular peptide preparation is an isolated or purified peptide can be confirmed, for example, by the appearance of a single band following sodium dodecyl sulfate (SDS)-polyacrylamide gel electrophoresis and Coomassie Brillliant Blue staining or such of the gel. In a preferred embodiment, the peptides and polynucleotides of the present invention are isolated or purified.

The terms “polypeptide”, “peptide”, “protein” and “protein” are used interchangeably herein, and refer to polymers of amino acid residues. These terms are applied to also non-naturally occurring amino acid polymers comprising one or more non-naturally occurring amino acid residues, in addition to naturally occurring amino acid polymers. Non-naturally occurring amino acids include amino acid analogs, amino acid mimetics, and such.

The term “amino acid” as used herein refers to naturally occurring amino acids, as well as amino acid analogs and amino acid mimetics that function similarly to the naturally occurring amino acids. Naturally occurring amino acids are those encoded by the genetic code, as well as those modified after translation in cells (e.g., hydroxyproline, gamma-carboxyglutamate, and O-phosphoserine, etc.). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine sulfoxide, methionine methyl sulfonium, and such). The phrase “amino acid mimetic” refers to chemical compounds that have different structures but similar functions to general amino acids. Amino acids can be either L-amino acids or D-amino acids, and the peptides of the present invention are preferably L-amino acid polymers.

The terms “polynucleotide”, “oligonucleotide” and “nucleic acid” are used interchangeably herein, and refer to a polymer of nucleotides.

The term “composition” used in the present specification is intended to encompass products that include specified ingredients in specified amounts, and any products generated directly or indirectly from combination of specified ingredients in the specified amounts. When the composition is a pharmaceutical composition, the term “composition” is intended to encompass products including active ingredient(s) and inert ingredient(s), as well as any products generated directly or indirectly from combination, complexation or aggregation of any two or more ingredients, from dissociation of one or more ingredients, or from other types of reactions or interactions of one or more ingredients. Thus, the pharmaceutical compositions of the present invention encompass any compositions made by admixing compounds or cells of the present invention with a pharmaceutically or physiologically acceptable carrier. Without being limited thereto, the terms “pharmaceutically acceptable carrier” or “physiologically acceptable carrier” used in the present specification include liquid or solid bulking agents, diluents, excipients, solvents, and encapsulation materials; and mean pharmaceutically or physiologically acceptable materials, compositions, substances or media.

Unless otherwise specified, the term “virus infectious disease” refers to a coronavirus infectious disease; and examples of coronaviruses include SARS-CoV-2, MERS-CoV, SARS-CoV and such, without being limited thereto. In an exemplary embodiment, the “coronavirus infectious disease” is SARS-CoV-2 infectious disease in an HLA-A24- or HLA-A02-positive subject.

Unless otherwise specified, the terms “cytotoxic T lymphocyte”, “cytotoxic T cell” and “CTL” are used interchangeably herein. Unless otherwise specifically indicated, they refer to a sub-group of T lymphocytes that can recognize non-self cells (for example, tumor/cancer cells, virus-infected cells) and induce the death of such cells.

Unless otherwise specified, the term “HLA-A24” refers to the HLA-A24 type which includes subtypes such as HLA-A*24:01, HLA-A*24:02, HLA-A*24:03, HLA-A*24:04, HLA-A*24:07, HLA-A*24:08, HLA-A*24:20, HLA-A*24:25 and HLA-A*24:88.

Unless otherwise specified, the term “HLA-A02 (or HLA-A2)” refers to the HLA-A02 type which includes subtypes such as HLA-A*02:01, HLA-A*02:02, A*02:03, A*02:04, A*02:05, A*02:06, A*02:07, A*02:10, A*02:11, A*02:13, A*02:16, A*02:18, A*02:19, A*02:28 and A*02:50.

Unless otherwise specified, the term “coronavirus protein” refers to a protein consisting of the full-length amino acid sequence of each protein encoded by coronavirus genome sequences. Preferred examples include structural and non-structural proteins of SARS-CoV-2. Moreover, examples of coronaviruses include, but are not limited to, SARS-CoV-2, MERS-CoV, and SARS-CoV. The genome sequence (reference sequence) of each of these coronaviruses and the amino acid sequences of coronavirus proteins encoded by it can be obtained with, for example, the following Genbank accession numbers:
SARS-CoV-2: MN908947
MERS-CoV: JX869059
SARS-CoV: Tor2: AY274119, BJ01: AY278488, or GZ02: AY390556

Unless otherwise specified, the term “SARS-CoV-2 protein” refers to a protein consisting of the full-length amino acid sequence of each protein encoded by the SARS-CoV-2 genome sequence, and includes the four structural proteins and six non-structural proteins that SARS-CoV-2 has. The four structural sequences and six non-structural proteins are listed in Table 1. In the table, as for the non-structural protein orflab, two ORFs in the reference sequence, 266 . . . 13468 and 13468 . . . 21555, are linked to form a single ORF by ribosomal frameshifting.

TABLE 1

| | | Reference | Coding | Amino |
|---|---|---|---|---|
| | | Sequence: | Sequence (CDS) | Acid |
| | | GenBank | in Genome | Sequence |
| | | accession | Sequence | (SEQ |
| | Protein | number | (MN908947.3) | ID NO) |
| Four | Spike Protein | QHD43416 | 21563 . . . 25384 | 17 |
| Structural | Envelope | QHD43418 | 26245 . . . 26472 | 18 |
| Proteins | Protein | | | |
| | Matrix Protein | QHD43419 | 26523 . . . 27191 | 19 |
| | Nucleoprotein | QHD43423 | 28274 . . . 29533 | 20 |
| Non- | ORF1ab | QHD43415 | 266 . . . | 21 |
| structural | | | 13468, 13468 . . . | |
| Six | | | 21555 | |
| Proteins | ORF3a | QHD43417 | 25393 . . . 26220 | 22 |
| | ORF6 | QHD43420 | 27202 . . . 27387 | 23 |
| | ORF7a | QHD43421 | 27394 . . . 27759 | 24 |
| | ORF8 | QHD43422 | 27894 . . . 28259 | 25 |
| | ORF10 | QHI42199 | 29558 . . . 29674 | 26 |

Unless otherwise specified, the terms “coronavirus-infected cells”, “virus-infected cells”, and “infected cells” are herein used interchangeably, and refer to cells infected with a coronavirus unless otherwise specifically indicated. Examples of coronaviruses include, but are not limited to, SARS-CoV-2, MERS-CoV, and SARS-CoV.

As used herein, the term “infection” includes viral entry and/or viral proliferation in cells or body tissues, and disease conditions resulting from viral entry and viral proliferation. The entry by viral life cycle and the proliferation stage of viral life cycle include, but are not limited to, binding of the viral particle to a cell, introduction of the viral genetic information into the cell, expression of viral proteins, production of new viral particles, and release of the viral particles from the cell.

In the context of a subject or patient, the phrase “HLA antigen of a subject (or patient) is HLA-A24 or HLA-A02” used herein refers to that a subject or patient has the HLA-A24 or HLA-A02 antigen gene homozygously or heterozygously as the MHC (Major Histocompatibility Complex) Class I molecule, and that the HLA-A24 or HLA-A02 antigen is expressed in the cells of the subject or patient as the HLA antigen.

As long as the methods and compositions of the present invention are useful in the context of coronavirus infectious disease “treatment”, the treatment is considered “efficacious” when it achieves clinical advantages, for example, alleviation of clinical symptoms of coronavirus infectious disease, suppression of aggravation in a subject. Fever, cough, chills, rigors, myalgia and the like are commonly known as main symptoms of coronavirus infectious diseases. It is considered that many infected patients have mild symptoms and recover within about one to two weeks. However, part of patients show symptoms of respiratory distress and may have marked dyspnea, hypoxia, or even acute respiratory distress syndrome (ARDS). According to recent reports, particularly in SARS-CoV-2-infected patients, unique symptoms such as dysosmia and dysgeusia have also been observed in patients with relatively mild symptoms. On the other hand, thrombi and cytokine storms in severely ill patients are known to result in aggravation. When treatment is applied prophylactically, the term “efficacious” means that the treatment retards or prevents development of a coronavirus infectious disease, or prevents or alleviates the clinical symptoms of a coronavirus infectious disease. Effectiveness is determined in relation to any publicly known method for diagnosing or treating a coronavirus infectious disease. For example, when any of the specific symptoms mentioned above is suppressed, the effectiveness of the treatment or prevention is indicated.

As long as the methods and compositions of the present invention are useful in the context of coronavirus infectious disease “prevention (prophylaxis)”, the term “prevention (prophylaxis)” herein includes any work that eases the load of disease-associated mortality or morbidity. Prevention (Prophylaxis) can be carried out at the “primary, secondary and tertiary prevention (prophylaxis) levels”. Whereas the primary prevention (prophylaxis) avoids the development of a disease, prevention (prophylaxis) at the secondary and tertiary levels encompasses prevention (prophylaxis) of disease progression and appearance of symptoms, as well as workings intended to reduce adverse effects of the existing disease by restoring functions and reducing disease-associated complications. Alternately, prevention (prophylaxis) can include alleviation of severity of a specific disorder, for example, extensive preventive therapy intended to reduce clinical symptoms such as fever, breathlessness, and upper respiratory infection.

In the context of the present invention, the treatment and/or prevention (prophylaxis) and/or the suppression of aggravation of coronavirus infectious diseases includes retarding the development of or ameliorating at least one symptom of coronavirus infectious diseases. It also includes inhibition of viral proliferation in coronavirus-infected cells. Effective treatment and/or prevention (prophylaxis) of coronavirus infectious diseases reduces mortality, improves the prognosis of individuals affected with coronavirus infectious diseases, and alleviates symptoms associated with coronavirus infectious diseases. For example, alleviation or improvement of symptoms constitutes effective treatment and/or prevention (prophylaxis), and includes 10%, 20%, 30% or more alleviation or stable conditions of symptoms.

In the present invention, inhibition of viral proliferation includes inhibition of the viral replication process in infected cells. When each viral protein expressed in the viral replication process is presented as an antigen in virus-infected cells, CTLs induced by the peptide of the present invention injure cellular functions (gene replication and protein translation) of the infected cells by their cytotoxic activity. Alternatively, the CTLs destroy the infected cells themselves. As a result, the replication process of viral particles, which relies on the cellular functions of the infected cells, is inhibited and viral proliferation is inhibited.

Furthermore, in the present invention, suppression of aggravation means suppression of the worsening or progression of any symptom associated with coronavirus infections. Specifically, the progression of respiratory distress symptoms that requires interventional treatment such as oxygen inhalation or placement of a ventilator or extracorporeal membrane oxygenation (ECMO) is a typical example of aggravation in coronavirus infectious diseases. Thus, stopping, prevention, or alleviation of the progression (worsening) of those symptoms is included in the suppression of aggravation in the present invention.

In the context of the present invention, the term “antibody” refers to immunoglobulins and fragments thereof that are specifically reactive to a designated protein or peptide thereof An antibody can include human antibodies, primatized antibodies, chimeric antibodies, bispecific antibodies, humanized antibodies, antibodies fused to other proteins or radiolabels, and antibody fragments. Furthermore, an “antibody” herein is used in the broadest sense and specifically covers intact monoclonal antibodies, polyclonal antibodies, multispecific antibodies (e.g., bispecific antibodies) formed from two or more intact antibodies, and antibody fragments so long as they exhibit the desired biological activity. An “antibody” may be antibodies of all classes (e.g., IgA, IgD, IgE, IgG and IgM).

The present invention relates to TCRs expressed by T cells that recognize a SARS-CoV-2-derived peptide. TCR is a protein molecule consisting of a dimer of an α chain and a β chain. Human T cells recognize peptides presented on MHC class I (also known as human leukocyte antigen, HLA) molecules through TCRs. As a result, T cell proliferation, differentiation, cytokine production, cytotoxic substance (perforin and granzymes) secretion and the like are induced.

The TCR-α gene comprises Vα, Jα and Cα genes. The TCR-β gene comprises Vβ, Dβ, Jβ and Cβ genes. The regions determining the specificity of TCR for a peptide are called complementarity determining regions (CDRs), which include CDR1, CDR2, and CDR3. Of these, CDR3 contacts a peptide directly, and therefore its amino acid sequence is very important in determining the antigen recognition specificity of TCR. In the TCR-α chain, V-J corresponds to CDR3, and in the TCR-β chain, V-D and D-J correspond to CDR3. Insertion and deletion of nucleotides generate their diversity.

In the present invention, a TCR refers to a protein molecule that is expressed on a T cell and consists of a dimer of an α chain and a β chain. In the present invention, a T cell is a human T cell and can be a human T cell that recognizes a SARS-CoV-2-derived peptide.

Furthermore, in the present invention, a TCR refers to a molecule that is expressed on a T cell, recognizes through itself a peptide presented on a MHC class I (also known as human leukocyte antigen, HLA) molecule, and induces, e.g., T cell proliferation, differentiation, cytokine production, and secretion of cytotoxic substances (perforin and granzymes).

The TCR-α gene comprises Vα, Jα and Cα genes. The TCR-β gene comprises Vβ, Dβ, Jβ and Cβ genes. The regions determining the specificity of TCR for a peptide are called complementarity determining regions (CDRs), which include CDR1, CDR2 and CDR3. Of these, CDR3 contacts a peptide directly, and therefore its amino acid sequence is very important in determining the antigen recognition specificity of TCR. In the TCR-α chain, V-J corresponds to CDR3, and in the TCR-β chain, V-D and D-J correspond to CDR3. Insertion and deletion of nucleotides generate their diversity.

In the present invention, a TCR is expressed on a T cell and may have CDR sequences that specifically recognize a SARS-CoV-2-derived peptide.

In the present invention, a “history of coronavirus infection” means that a subject has been previously infected with a coronavirus, particularly SARS-CoV-2. Even when a subject was previously infected but asymptomatic, the previous infection can be confirmed by detecting the TCRs of the present invention. Moreover, a subject who is currently infected means that the infection was previously established. Thus, a subject who is currently infected has a previous history of coronavirus infection. COVID-19 means a “novel coronavirus infectious disease (COVID-19 infection)” definitively diagnosed by physicians.

Unless otherwise specified, the technical terms and scientific terms used herein all have the same meanings as terms commonly understood by one of ordinary skill in the art to which the present invention belongs.

### II. Peptides

HLA-A24 is an allele commonly seen in Asians, and HLA-A02 is an allele commonly seen in Caucasians (Sette A, Sidney J., Immunogenetics 1999, 50: 201-12; Cao K et al., Hum Immunol 2001, 62(9): 1009-1030; Gonzalez-Galarza F F et al., Nucleic Acids Res 2020, 48(D1): D783-D788). Thus, an effective method of treating coronavirus infectious diseases for a great population of Asians or Caucasians can be provided by providing SARS-CoV-2 protein-derived

CTL-inducing peptides restricted to HLA-A24 or HLA-A02. Thus, the present invention provides SARS-CoV-2 protein-derived peptides that are capable of inducing CTLs in an HLA-A24- or HLA-A02-restrictive manner.

The peptides of the present invention are SARS-CoV-2 protein-derived peptides that are capable of inducing CTLs in an HLA-A24- or HLA-A02-restrictive manner. Peptides capable of inducing CTLs in an HLA-A02-restrictive manner include peptides having the amino acid sequence selected from among SEQ ID NOs: 1, 2, 3, 4, 5, 7, 9, 10, 11, 12, 13, 14 and 15. Peptides capable of inducing CTLs in an HLA-A24-restrictive manner include peptides having the amino acid sequence selected from among SEQ ID NOs: 3 and 11.

CTLs having a cytotoxic activity specific to these peptides can be established by in vitro stimulation of T cells by dendritic cells pulsed with these peptides. The established CTLs show a specific cytotoxic activity against target cells pulsed with each of the peptides.

CTLs are induced following presentation of coronavirus antigens (epitope peptides derived from coronavirus proteins) and then recognize epitope peptides presented on human leukocyte antigen (HLA) class I molecules expressed on the surface of coronavirus-infected cells to kill these cells. Thus, coronavirus antigens are excellent targets for immunotherapy. Therefore, the peptides of the present invention can be suitably used for immunotherapy of coronavirus infectious diseases. A preferred peptide is a nonapeptide (a peptide consisting of 9 amino acid residues) or a decapeptide (a peptide consisting of 10 amino acid residues), and it is more preferably a peptide consisting of the amino acid sequence selected from among SEQ ID NOs: 1, 2, 3, 4, 5, 7, 9, 10, 11, 12, 13, 14 and 15. For example, a peptide having the amino acid sequence of SEQ ID NO: 1, 2, 3, 4, 5, 7, 9, 10, 11, 12, 13, 14 or 15 is suitable for induction of CTLs that show a specific cytotoxic activity against coronavirus-infected cells having HLA-02, and can be suitably used for immunotherapy of coronavirus infectious disease for HLA-A02-positive patients. Furthermore, for example, a peptide having the amino acid sequence of SEQ ID NO: 3 or 11 is suitable for induction of CTLs that show a specific cytotoxic activity against coronavirus-infected cells having HLA-24, and can be suitably used for immunotherapy of coronavirus infectious disease for HLA-A24-positive patients. In a more preferred embodiment, the peptide of the present invention is a peptide consisting of the amino acid sequence selected from among SEQ ID NOs: 1, 2, 3, 4, 5, 7, 9, 10, 11, 12, 13, 14 and 15 for HLA-A02-positive patients, and a peptide consisting of the amino acid sequence selected from among SEQ ID NOs: 3 and 11 for HLA-A24-positive patients.

For the peptides of the present invention, an additional amino acid residue(s) can be made to adjoin the amino acid sequence of the peptide of the present invention, as long as the resultant peptides retain the CTL-inducing ability of the original peptide. The additional amino acid residue(s) may be composed of any types of amino acid(s), as long as they do not impair the CTL-inducing ability of the original peptide. Therefore, the peptides of the present invention encompass peptides having CTL-inducing ability, comprising the amino acid sequence selected from among SEQ ID NOs: 1, 2, 3, 4, 5, 7, 9, 10, 11, 12, 13, 14 and 15. Such peptides are, for example, less than about 40 amino acids, in many cases less than about 20 amino acids, and usually less than about 15 amino acids. Therefore, if the original peptide is a nonapeptide, the peptide of the present invention encompasses peptides that are 10 amino-acid long or 11-40 amino-acid long, which are produced by adjoining additional amino acid(s) to the peptide. Furthermore, if the original peptide is a decapeptide, the peptide of the present invention encompasses peptides that are 11-40 amino-acid long. Such a peptide can be, for example, a peptide that is 11-20 amino-acid long or a peptide that is 11-15 amino-acid long. A preferred example of an additional amino acid residue(s) is an amino acid residue(s) adjacent to the amino acid sequence of the peptide of the present invention in the full-length amino acid sequence of each protein encoded by a genome sequence of SARS-CoV-2 (for example, SEQ ID NOs: 17-26). Therefore, the peptides of the present invention encompass peptides comprising the amino acid sequence selected from among SEQ ID NOs: 1, 2, 3, 4, 5, 7, 9, 10, 11, 12, 13, 14 and 15, and wherein the peptides are peptide fragments of SARS-CoV-2 protein and have CTL-inducing ability.

In general, modifications of one, two or more amino acids in a certain peptide do not affect the functions of the peptide, or in some cases even enhance the desired functions of the original peptide. In fact, modified peptides (i.e., peptides composed of the amino acid sequence in which one, two or several amino acid residues are modified (i.e., substituted, deleted, inserted, and/or added) compared to the original reference sequence) are known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the peptides of the present invention can be peptides comprising the amino acid sequence in which one, two or several amino acids are substituted, deleted, inserted and/or added to the amino acid sequence selected from among SEQ ID NOs: 1, 2, 3, 4, 5, 7, 9, 10, 11, 12, 13, 14 and 15 and having CTL-inducing ability.

One skilled in the art can recognize that individual substitutions to an amino acid sequence that alter a single amino acid or a small percentage of amino acids tend to result in the conservation of the properties of the original amino acid side chain(s). Accordingly, those are frequently referred to as “conservative substitutions” or “conservative modifications”; and modification of a protein by “conservative substitution” or “conservative modification” may result in a modified protein that has similar functions as the original protein. Tables of conservative substitutions presenting functionally similar amino acids are well known in the art. Examples of amino acid side chain characteristics that functionally resemble include, for example, hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins 1984).

Such conservatively modified peptides are also encompassed in peptides of the present invention. However, peptides of the present invention are not restricted thereto and can include non-conservative modifications, so long as the modified peptide retains the CTL-inducing ability of the original peptide. Furthermore, modified peptides do not exclude CTL inducible peptides derived from polymorphic variants, interspecies homologues, and alleles of SARS-CoV-2 proteins.

So long as a peptide retains the CTL-inducing ability of an original peptide, one can modify (i.e., substitute, delete, insert and/or add) a small number (for example, 1, 2 or several) or a small percentage of amino acids. Herein, the term “several” means 5 or fewer amino acids, for example, 4 or 3 or fewer. The percentage of amino acids to be modified is preferably 20% or less, more preferably 15% or less, even more preferably 10% or less or 1 to 5%.

When used in the context of immunotherapy, peptides of the present invention should be presented on the surface of a cell or exosome, preferably as a complex with an HLA antigen. Therefore, it is preferable that the peptides of the present invention possess high binding affinity to the HLA antigen. To that end, the peptides can be modified by substitution, deletion, insertion, and/or addition of the amino acid residues to yield a modified peptide having improved binding affinity. Since the regularity of the sequences of peptides displayed by binding to HLA antigens is already known (Falk, et al., Immunogenetics 1994 40 232-41; Chuj oh, et al., Tissue Antigens 1998: 52: 501-9; Takiguchi, et al., Tissue Antigens 2000: 55: 296-302.), modifications based on such regularity can be introduced into the peptides of the present invention.

For example, in peptides having binding affinity for HLA Class I, the second amino acid from the N terminus and the C-terminal amino acid are generally anchor residues involved in the binding to HLA Class I (Rammensee H G, et al., Immunogenetics. 1995; 41(4): 178-228.).

For example, peptides having high HLA-A02-binding affinity tend to have the second amino acid from the N terminus substituted with leucine or methionine and/or the C-terminal amino acid substituted with valine or leucine. Thus, in order to enhance HLA-A02-binding affinity, it may be desirable to substitute the second amino acid from the N terminus with leucine or methionine and/or to substitute the C-terminal amino acid with valine or leucine. Accordingly, the present invention encompasses peptides having an amino acid sequence selected from those of SEQ ID NOs: 1, 2, 3, 4, 5, 7, 9, 10, 11, 12, 13, 14 and 15 in which the second amino acid from the N terminus has been substituted with leucine or methionine and/or the C-terminus has been substituted with valine or leucine.

Similarly, the present invention encompasses peptides comprising an amino acid sequence in which one, two, or several amino acids are substituted, deleted, inserted, and/or added in the sequences of SEQ ID NOs: 1, 2, 3, 4, 5, 7, 9, 10, 11, 12, 13, 14 and 15, which peptides have one or both of the features: (a) the second amino acid from the N terminus is leucine or methionine; and (b) the C-terminal amino acid is valine or leucine. In a preferred embodiment, the peptides of the present invention comprise an amino acid sequence comprising one or both of the substitution of the second amino acid from the N terminus with leucine or methionine; and the substitution of the C-terminal amino acid with valine or leucine, in the amino acid sequences of SEQ ID NOs: 1, 2, 3, 4, 5, 7, 9, 10, 11, 12, 13, 14 and 15.

For example, among peptides having high HLA-A02-binding affinity, peptides having high HLA-A*02:06-binding affinity tend to have the second amino acid from the N terminus substituted with valine or glutamine and/or the C-terminal amino acid substituted with valine or leucine. Thus, in order to enhance HLA-A*02:06-binding affinity, it may be desirable to substitute the second amino acid from the N terminus with valine or glutamine and/or to substitute the C-terminal amino acid with valine or leucine.

Accordingly, the present invention encompasses peptides having an amino acid sequence selected from those of SEQ ID NOs: 3, 4, 7, 10, 11, 12 and 15 in which the second amino acid from the N terminus has been substituted with valine or glutamine and/or the C-terminus has been substituted with valine or leucine (WO 2010/047310, Table 1).

Similarly, the present invention encompasses peptides comprising an amino acid sequence in which one, two, or several amino acids are substituted, deleted, inserted, and/or added in the sequences of SEQ ID NOs: 3, 4, 7, 10, 11, 12 and 15, which peptides have one or both of the features: (a) the second amino acid from the N terminus is valine or glutamine; and (b) the C-terminal amino acid is valine or leucine. In a preferred embodiment, the peptides of the present invention comprise an amino acid sequence comprising one or both of the substitution of the second amino acid from the N terminus with valine or glutamine; and the substitution of the C-terminal amino acid with valine or leucine, in the amino acid sequences of SEQ ID NOs: 3, 4, 7, 10, 11, 12 and 15.

For example, peptides having high HLA-A24-binding affinity tend to have the second amino acid from the N terminus substituted with phenylalanine, tyrosine, methionine, or tryptophan. Similarly, peptides in which the C-terminal amino acid has been substituted with phenylalanine, leucine, isoleucine, tryptophan, or methionine tend to have high HLA-A24-binding affinity. Thus, in order to enhance HLA-A24-binding affinity, it may be desirable to substitute the second amino acid from the N terminus with phenylalanine, tyrosine, methionine, or tryptophan and/or to substitute the C-terminal amino acid with phenylalanine, leucine, isoleucine, tryptophan, or methionine. Accordingly, the present invention encompasses peptides having an amino acid sequence selected from those of SEQ ID NOs: 3 and 11 in which the second amino acid from the N terminus has been substituted with phenylalanine, tyrosine, methionine or tryptophan and/or the C terminus has been substituted with phenylalanine, leucine, isoleucine, tryptophan, or methionine.

Similarly, the present invention encompasses peptides comprising an amino acid sequence in which one, two, or several amino acids are substituted, deleted, inserted, and/or added in the sequences of SEQ ID NOs: 3 and 11, which peptides have one or both of the features: (a) the second amino acid from the N terminus is phenylalanine, tyrosine, methionine or tryptophan; and (b) the C-terminal amino acid is phenylalanine, leucine, isoleucine, tryptophan or methionine. In a preferred embodiment, the peptides of the present invention comprise an amino acid sequence comprising one or both of the substitution of the second amino acid from the N terminus with phenylalanine, tyrosine, methionine or tryptophan; and the substitution of the C-terminal amino acid with phenylalanine, leucine, isoleucine, tryptophan or methionine, in the amino acid sequences of SEQ ID NOs: 3 and 11.

Substitution(s) may be introduced into amino acid(s) not only at the terminus/termini, but also at a position(s) of potential T cell receptor (TCR) recognition site(s) of the peptides. Several research studies have demonstrated that a peptide that has amino acid substitutions, such as CAP1, p53₍₂₆₄₋₂₇₂₎, Her-2/neu₍₃₆₉₋₃₇₇₎ or gp100₍₂₀₉₋₂₁₇₎, may have equal to or better activity than that of the original peptide (Zaremba et al. Cancer Res. 57, 4570-4577, 1997; T. K. Hoffmann et al. J Immunol. (2002) Feb 1, 168(3): 1338-47; S. 0. Dionne et al. Cancer Immunol immunother. (2003) 52: 199-206; and S. O. Dionne et al. Cancer Immunology, Immunotherapy (2004) 53, 307-14).

The present invention also contemplates that one, two or several amino acids can be added to the N terminus and/or C terminus of the peptides of the present invention (for example, peptides consisting of the amino acid sequence selected from among SEQ ID NOs: 1, 2, 3, 4, 5, 7, 9, 10, 11, 12, 13, 14 and 15). Such modified peptides that retain CTL-inducing ability are also included in the present invention. For example, when a peptide in which one, two or several amino acids are added to the N terminus and/or C terminus of a peptide consisting of the amino acid sequence of SEQ ID NO: 1, 2, 3, 4, 5, 7, 9, 10, 11, 12, 13, 14 or 15 is contacted with an APC(s), it is incorporated into the APC(s) and processed to become a peptide consisting of the amino acid sequence of SEQ ID NO: 1, 2, 3, 4, 5, 7, 9, 10, 11, 12, 13, 14 or 15. It can then induce CTLs through presentation on the cell surface of an APC via the antigen presentation pathway.

However, when the amino acid sequence of a peptide is identical to a portion of the amino acid sequence of an endogenous or exogenous protein having a different function, side effects such as autoimmune disorders and/or allergic symptoms against specific substances may be induced. Therefore, it is preferable to perform homology searches using available databases to avoid situations in which the amino acid sequence of the peptide matches the amino acid sequence of another protein. When it becomes clear from the homology searches that no peptide exists with as few as 1 or 2 amino acid differences as compared to the objective peptide, the objective peptide can be modified in order to increase its binding affinity with HLA antigens, and/or increase its CTL-inducing ability without danger of such side effects.

Peptides in which one, two or several amino acids of a peptide of the present invention are modified are predicted to be able to retain CTL-inducing ability of the original peptide; however, it is preferable to verify the CTL-inducing ability of the modified peptides. Herein, the “peptide having CTL-inducing ability (CTL inducibility)” refers to a peptide that induces CTLs through APCs stimulated with the peptide. “CTL induction” includes induction of differentiation into CTLs, induction of CTL activation, induction of CTL proliferation, induction of CTL's cytotoxic activity, induction of CTL-mediated dissolution of target cells, and induction of increase of IFN-gamma production of CTLs.

The CTL-inducing ability can be confirmed by inducing and stimulating APCs that retain an HLA antigen (for example, B lymphocytes, macrophages, and dendritic cells) with a peptide, and mixing it with CD8-positive T cells; and then measuring IFN-gamma released by CTLs against the target cells. For the APCs, human peripheral blood mononuclear leukocyte-derived dendritic cells can be preferably used. As a reaction system, transgenic animals generated to express an HLA antigen can be used. Alternatively, for example, the target cells may be radio-labelled with ⁵¹Cr or such, and the cytotoxic activity of the peptide-induced CTLs may be calculated from the radioactivity emitted from the target cells. Alternatively, in the presence of peptide-stimulated APCs, it is possible to evaluate the CTL-inducing ability by measuring the IFN-gamma produced and released by CTLs, and visualizing the inhibition zone on the media using anti-IFN-gamma monoclonal antibodies.

In addition to the modifications above, the peptides of the present invention can be linked to other peptides as long as the resultant linked peptide retains the CTL-inducing ability. An example of an appropriate peptide to be linked with the peptides of the present invention includes CTL-inducing peptide derived from other coronavirus proteins. Further, the peptides of the present invention can also be linked with each other. Suitable linkers for use in linking peptides are known in the art, and for example, linkers such as AAY (P. M. Daftarian et al., J Trans Med 2007, 5:26), AAA, NKRK (SEQ ID NO: 27) (R. P. M. Sutmuller et al., J Immunol. 2000, 165: 7308-15), or K (S. Ota et al., Can Res. 62, 1471-6, K. S. Kawamura et al., J Immunol. 2002, 168: 5709-15) can be used. Peptides can be linked in various arrangements (for example, catenulate, repeated, etc.), and one can also link three or more peptides.

The peptides of the present invention can also be linked to other substances as long as the resultant linked peptide retains the CTL-inducing ability. Examples of an appropriate substance to be linked with a peptide of the present invention include, for example, a peptide, a lipid, a sugar or sugar chain, an acetyl group, and a naturally-occurring or synthetic polymer. The peptides of the present invention can be modified by glycosylation, side-chain oxidation, phosphorylation or such, as long as their CTL-inducing ability is not impaired. One can also perform such types of modifications to confer additional functions (for example, targeting function and delivery function) or to stabilize the peptide.

For example, to increase the in vivo stability of a peptide, it is known in the art to introduce D-amino acids, amino acid mimetics or non-naturally occurring amino acids, and this concept may also be applied to peptides of the present invention. Peptide stability can be assayed by several methods. For example, stability can be tested by using a peptidase as well as various biological media such as human plasma and serum (see, e.g., Verhoef et al., Eur J Drug Metab Pharmacokin 1986, 11: 291-302).

Further, as stated above, among the modified peptides in which one, two, or several amino acid residues have been substituted, deleted, inserted and/or added, those having the same or higher activity as compared to original peptides can be screened for or selected. Thus, the present invention also provides methods of screening for or selecting modified peptides that have the same or higher activity than that of the original peptide. Specifically, the present invention provides a method of screening for a peptide having CTL-inducing ability, wherein the method comprises the steps of:
(a) generating candidate sequences consisting of an amino acid sequence in which one, two, or several amino acid residues are substituted, deleted, inserted and/or added to the original amino acid sequence consisting of the amino acid sequence selected from among SEQ ID NOs: 1, 2, 3, 4, 5, 7, 9, 10, 11, 12, 13, 14 and 15;
(b) selecting from among the candidate sequences generated in (a), a candidate sequence that does not have a significant homology (sequence identity) with any known human gene product;
(c) contacting a peptide consisting of the candidate sequence selected in (b) with APCs;
(d) contacting the APCs of (c) with CD8-positive T cells; and
(e) selecting a peptide that has an equal to or higher CTL-inducing ability than that of a peptide consisting of the original amino acid sequence.

In an embodiment, APCs to be contacted with a peptide are APCs positive for either one of HLA-A02 or HLA-A24, or for both.

Herein, the peptide of the present invention is also described as a “SARS-CoV-2 peptide(s)” or a “SARS-CoV-2 polypeptide(s)”.

In a certain embodiment of the present invention, a portion of TCR can also comprise one, two, or three complementarity determining regions (CDRs) of one or both of the α and β chains of the TCR. In a preferred embodiment, the portion of TCR comprises CDR3 of one or both of the α and β chains of the TCR. The amino acid sequences of the preferred CDR3 identified in the present invention are as follows:

CDR3 of a human T cell receptor α chain specified by any one of the amino acid sequences selected from the group consisting of SEQ ID NOs: 32, 34, 36 and 38; and

CDR3 of a human T cell receptor β chain specified by any one of the amino acid sequences selected from the group consisting of SEQ ID NOs: 33, 35, 37 and 39.

In a certain embodiment of the present invention, the amino acid sequences of CDR3 of T cell receptor α and β chains can be combined, for example, as follows:

CDR3 of T cell receptor α chain CDR3 of T cell receptor β chain
SEQ ID NO: 32 SEQ ID NO: 33;
SEQ ID NO: 34 SEQ ID NO: 35;
SEQ ID NO: 36 SEQ ID NO: 37; and
SEQ ID NO: 38 SEQ ID NO: 39.

Furthermore, CDR3s comprising conservative modifications are also included in the CDR3s in the present invention. However, the peptides of the present invention are not limited thereto, and may comprise non-conservative modifications as long as the modified CDR3 retains the function of the original CDR3.

In the present invention, when the same antigen recognition specificity as that of the TCR from which the original CDR3 is derived is conferred on a TCR to which a modified CDR3 is transplanted, it means that the original function of the CDR3 is retained in the modified CDR3. Such specific recognition can be confirmed by any known method, and preferred methods include, for example, tetramer assays using peptides administered to a subject who has acquired HLA molecules and TCRs (e.g., Altman et al. Science. 1996, 274, 94-6; McMichael et al. J Exp Med. 1998, 187, 1367-71) and ELISPOT assays. It can be confirmed by performing an ELISPOT assay that T cells expressing TCRs on their cell surface recognize cells by the TCRs, and that signals are transduced intracellularly and then cytokines such as IFN-γ are released from the T cells. Cytotoxic activity of T cells against target cells can be examined using methods well known in the art. Preferred methods include, for example, a chromium release assay using coronavirus-infected HLA-positive cells as a target cell.

### III. Preparation of Peptides of the Present Invention

Well known techniques can be used to prepare peptides of the present invention. For example, recombinant DNA technology or chemical synthesis can be used to prepare peptides of the present invention. Peptides of the present invention can be synthesized individually, or as longer polypeptides including two or more peptides. Peptides of the present invention can be isolated from host cells or synthesis reaction products after they are produced in the host cells using recombinant DNA technology or after they are chemically synthesized. That is, peptides of the present invention can be purified or isolated so as not to substantially contain other host-cell proteins and fragments thereof, or any other chemical substances.

The peptides of the present invention may contain modifications, such as glycosylation, side chain oxidation, or phosphorylation provided such modifications do not destroy the biological activity of the original peptide. Other illustrative modifications include incorporation of D-amino acids or other amino acid mimetics that may be used, for example, to increase the serum half life of the peptides.

A peptide of the present invention can be obtained through chemical synthesis based on the selected amino acid sequence. Examples of conventional peptide synthesis methods that can be adapted to the synthesis include the methods described in the documents below:
(i) Peptide Synthesis, Interscience, New York, 1966;
(ii) The Proteins, Vol. 2, Academic Press, New York, 1976;
(iii) “Peptide Synthesis” (in Japanese), Maruzen Co., 1975;
(iv) “Basics and Experiment of Peptide Synthesis” (in Japanese), Maruzen Co., 1985;
(v) “Development of Pharmaceuticals” (in Japanese), Continued Vol. 14 (peptide synthesis), Hirokawa, 1991;
(vi) WO99/67288; and
(vii) Barany G. & Merrifield R. B., Peptides Vol. 2, Solid Phase Peptide Synthesis, Academic Press, New York, 1980, 100-18.

Alternatively, the peptides of the present invention can be obtained adapting any known genetic engineering methods for producing peptides (e.g., Morrison J, J Bacteriology 1977, 132: 349-51; Clark-Curtiss & Curtiss, Methods in Enzymology (Wu et al.) 1983, 101: 347-62). For example, first, a suitable vector harboring a polynucleotide encoding the peptide of the present invention in an expressible form (e.g., downstream of a regulatory sequence corresponding to a promoter sequence) is prepared and transformed into a suitable host cell. The host cell is then cultured to produce the peptide of the present invention. The peptide of the present invention can also be produced in vitro using an in vitro translation system.

### IV. Polynucleotides

The present invention also provides a polynucleotide which encodes any of the peptides of the present invention. These include polynucleotides derived from the naturally occurring SARS-CoV-2 gene (e.g., GenBank Accession No. MN908947 (SEQ ID NO: 16)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described above without altering the encoded polypeptide. Such nucleic acid variations are “silent variations”, which are one species of conservatively modified variations. Every nucleic acid sequence herein which encodes a peptide also describes every possible silent variation of the nucleic acid. One of ordinary skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence.

The polynucleotide of the present invention can be composed of DNA, RNA, and derivatives thereof. A DNA is suitably composed of bases such as A, T, C and G, and T is replaced by U in an RNA.

The polynucleotide of the present invention can encode multiple peptides of the present invention with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (e.g., plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage SL & Iyer RP, Tetrahedron 1992, 48: 2223-311; Matthes et al., EMBO J 1984, 3: 801-5.

### V. Exosomes

The present invention further provides intracellular vesicles, referred to as exosomes, that present complexes formed between the peptides of the present invention and HLA antigens on their surface. Exosomes can be prepared, for example, using the methods detailed in JPH11-510507 and WO99/03499, and can be prepared using APCs obtained from patients who are subject to treatment and/or prevention (prophylaxis). The exosomes of the present invention can be inoculated as vaccines, in a fashion similar to the peptides of the present invention.

The type of the HLA antigens included in the above-described complexes must match that of the subject in need of treatment and/or prevention (prophylaxis). For example, HLA-A24 (for example, HLA-A*24:02) is an allele widely and generally seen in Asian countries including Japan, and HLA-A02 (for example, HLA-A*02:01 or HLA-A*02:06) is an allele widely and generally seen in Europe and the United States. These HLA antigen types are considered to be suitable for treatment in Asian or Caucasian patients. Typically in clinic, it is possible to select an appropriate peptide that has a high level of binding affinity for a specific HLA antigen or that has CTL-inducing ability by antigen presentation mediated by a specific HLA antigen, by studying in advance the HLA antigen type of the patient in need of treatment.

The exosomes of the present invention present on their surface a complex of a peptide of the present invention and HLA-A24 or HLA-A02. When the HLA that forms a complex with a peptide of the present invention is HLA-A02, the peptide of the present invention is preferably a peptide having the amino acid sequence selected from among SEQ ID NOs: 1, 2, 3, 4, 5, 7, 9, 10, 11, 12, 13, 14 and 15 or a modified peptide thereof, and more preferably a peptide consisting of the amino acid sequence selected from among SEQ ID NOs: 1, 2, 3, 4, 5, 7, 9, 10, 11, 12, 13, 14 and 15 or a modified peptide thereof.

When the HLA that forms a complex with a peptide of the present invention is HLA-A24, the peptide of the present invention is preferably a peptide having the amino acid sequence selected from among SEQ ID NOs: 3 and 11 or a modified peptide thereof, and more preferably a peptide consisting of the amino acid sequence selected from among SEQ ID NOs: 3 and 11 or a modified peptide thereof.

### VI. Antigen-Presenting Cells (APCs)

The present invention further provides APCs that present on their surface complexes formed between HLA antigens and peptides of the present invention. Alternatively, the present invention provides APCs having on their cell surface complexes formed between HLA antigens and peptides of the present invention. The APCs of the present invention can be isolated APCs. When used in the context of cells (APCs, CTLs, etc.), the term “isolated” means that the cells are separated from another type of cells. The APCs of the present invention may be APCs induced from APCs derived from the patient to be subjected to treatment and/or prevention (prophylaxis), and can be administered as a vaccine by themselves or in combination with other drugs including a peptide(s), an exosome(s) or a CTL(s) of the present invention.

The APCs of the present invention are not limited to specific types of cells, and include cells known to present proteinaceous antigens on their cell surface so as to be recognized by lymphocytes, for example, dendritic cells (DCs), Langerhans cells, macrophages, B cells, and activated T cells. Since DC is a representative APC that has the strongest CTL-inducing activity among APCs, DCs can be preferably used as the APCs of the present invention.

For example, APCs of the present invention can be obtained by inducing DCs from peripheral blood monocytes and then stimulating them in vitro, ex vivo, or in vivo with the peptides of the present invention. When the peptide of the present invention is administered to a subject, APCs presenting the peptide of the present invention are induced in the body of the subject. Therefore, after the peptides of the present invention are administered to a subject, the APCs of the present invention can be obtained by collecting APCs from the subject. Alternatively, the APCs of the present invention can be obtained by contacting APCs collected from a subject with a peptide of the present invention.

In order to induce an immune response against coronavirus-infected cells in a subject, the APCs of the present invention can be administered to the subject by themselves or in combination with other drugs including peptide(s), exosome(s) or CTL(s) of the present invention. For example, the ex vivo administration can comprise the following steps of:
(a) collecting APCs from a first subject;
(b) contacting the APCs of step (a) with a peptide; and
(c) administering the APCs of step (b) to a second subject.

The first subject and the second subject may be the same individual, or may be different individuals. When the first subject and the second subject are different individuals, it is preferable that the HLAs of the first subject and the second subject are of the same type. The APC obtained in step (b) above can be a vaccine for treatment and/or prevention (prophylaxis) of coronavirus infectious disease. In a certain mode, the methods of the present invention may further additionally comprise the step of collecting the APCs after step (b). The collected APCs may then be administered to a second subject.

The APCs of the present invention obtained by a method such as described above have CTL-inducing ability. The term “CTL-inducing ability (CTL inducibility)” used in the context of an APC(s) refers to the ability of the APC to be able to induce a CTL(s) when placed in contact with a CD8-positive T cell(s). The CTL(s) induced by the APC of the present invention is a CTL(s) specific to SARS-CoV-2 protein and demonstrates a specific cytotoxic activity against SARS-CoV-2 infected cells.

In addition to the above-described methods, the APCs of the present invention can be prepared by introducing a polynucleotide encoding a peptide of the present invention into APCs in vitro. The polynucleotide to be introduced can be in the form of DNA or RNA. The method of introduction is not particularly limited, and examples thereof include various methods conventionally performed in the art such as lipofection, electroporation and the calcium phosphate method. More specifically, methods described in Cancer Res 1996, 56: 5672-7; J Immunol 1998, 161: 5607-13; J Exp Med 1996, 184: 465-72, and JP2000-509281 can be used. By introducing a polynucleotide encoding a peptide of the present invention into an APC, the polynucleotide is transcribed and translated in the cell, and then the produced peptide is processed by MHC Class I and proceeds through a presentation pathway to present the peptide of the present invention on the cell surface of the APC.

In a preferred embodiment, the APC of the present invention is an APC that presents on its cell surface a complex formed between a peptide of the present invention and HLA-A02 (more preferably HLA-A*02:01 or HLA-A*02:06). When the HLA that forms a complex with a peptide of the present invention is HLA-A02, the peptide of the present invention is preferably a peptide having the amino acid sequence selected from among SEQ ID NOs: 1, 2, 3, 4, 5, 7, 9, 10, 11, 12, 13, 14 and 15 or a modified peptide thereof, and more preferably a peptide consisting of the amino acid sequence selected from among SEQ ID NOs: 1, 2, 3, 4, 5, 7, 9, 10, 11, 12, 13, 14 and 15.

Alternatively, in a preferred embodiment, the APC of the present invention is an APC that presents on its cell surface a complex formed between a peptide of the present invention and HLA-A24 (more preferably HLA-A*24:02). When the HLA that forms a complex with a peptide of the present invention is HLA-A24, the peptide of the present invention is preferably a peptide having the amino acid sequence selected from among SEQ ID NOs: 3 and 11 or a modified peptide thereof, and more preferably a peptide consisting of the amino acid sequence selected from among SEQ ID NOs: 3 and 11.

The APC(s) of the present invention is preferably an APC(s) induced by a method comprising a step described (a) or (b) below:
(a) contacting an APC(s) expressing HLA-A02 (more preferably HLA-A*02:01 or HLA-A*02:06) or HLA-A24 (more preferably HLA-A*24:02) with a peptide of the present invention; or
(b) introducing a polynucleotide encoding a peptide of the present invention into an APC(s) expressing HLA-A02 (more preferably HLA-A*02:01 or HLA-A*02:06) or HLA-A24 (more preferably HLA-A*24:02).

The peptide of the present invention to be contacted with the HLA-A02-expressing APC(s) is preferably a peptide having the amino acid sequence selected from among SEQ ID NOs: 1, 2, 3, 4, 5, 7, 9, 10, 11, 12, 13, 14 and 15 or a modified peptide thereof, and more preferably a peptide consisting of the amino acid sequence selected from among SEQ ID NOs: 1, 2, 3, 4, 5, 7, 9, 10, 11, 12, 13, 14 and 15. The peptide of the present invention to be contacted with the HLA-A24-expressing APC(s) is preferably a peptide having the amino acid sequence selected from among SEQ ID NOs: 3 and 11 or a modified peptide thereof, and more preferably a peptide consisting of the amino acid sequence selected from among SEQ ID NOs: 3 and 11.

The present invention provides use of a peptide of the present invention for the manufacture of a pharmaceutical composition that induces an APC(s) having CTL-inducing ability. In addition, the present invention provides a method or process of manufacturing a pharmaceutical composition that induces an APC(s) having CTL-inducing ability. Further, the present invention provides a peptide of the present invention for inducing an APC(s) having CTL-inducing ability.

VII. Cytotoxic T lymphocytes (CTLs)

The CTL induced by a peptide of the present invention can be used as a vaccine in a similar manner to the peptide of the present invention for enhancing an immune response targeting coronavirus-infected cell in vivo. Thus, the present invention provides CTLs that are induced or activated by a peptide of the present invention. The CTLs of the present invention are CTLs that target a peptide of the present invention, and are capable of binding to a complex of a peptide of the present invention and an HLA antigen. Binding of a CTL to the complex is mediated via a T cell receptor (TCR) present on the cell surface of the CTL. The CTLs of the present invention can be isolated CTLs.

The CTLs of the present invention can be obtained by (1) administering a peptide of the present invention to a subject, (2) stimulating APCs and CD8-positive T cells, or peripheral blood mononuclear cells (PBMCs) derived from a subject with a peptide of the present invention in vitro, (3) contacting in vitro CD8-positive T cells or PBMCs with APCs or exosomes that present on their surface a complex of an HLA antigen and a peptide of the present invention, or (4) introducing into CD8-positive T cells a vector comprising a polynucleotide encoding each subunit of a T cell receptor (TCR) capable of binding to a peptide of the present invention presented on cell surface via an HLA antigen. The exosomes and APCs used in the method of (2) or (3) above can be prepared by methods described in the “V. Exosomes” and “VI. Antigen-presenting cells (APCs)” sections, respectively, and the details of the method of (4) above will be described in the “VIII. T cell receptor (TCR)” section. In a certain embodiment, the methods of the present invention may further additionally comprise the step of collecting induced CTLs after each step.

The CTLs of the present invention can be administered by themselves to patients who are subject to treatment and/or prevention (prophylaxis), or in combination with other drugs including peptide(s), APC(s) or exosome(s) of the present invention for the purpose of regulating effects. Further, the CTLs of the present invention can be CTLs induced from CD8-positive T cells derived from the patients who are subject to administration of the CTLs. The CTLs of the present invention act specifically on target cells that present the peptides of the present invention, for example, the same peptides used to induce the CTLs of the present invention. The target cells may be cells that endogenously express SARS-CoV-2 protein, such as coronavirus-infected cells, or cells transfected with the SARS-CoV-2 gene. Cells that present a peptide of the present invention on their cell surface due to stimulation by the peptide can become a target of attack by the CTLs of the present invention. The cells targeted by the CTLs of the present invention are preferably cells that are positive for HLA-A02 (more preferably HLA-A*02:01 or HLA-A*02:06) or HLA-A24 (more preferably HLA-A*24:02).

In a preferred embodiment, the CTLs of the present invention target specifically cells that express both SARS-CoV-2 protein and HLA-A02 (more preferably HLA-A*02:01 or HLA-A*02:06) or HLA-A24 (more preferably HLA-A*24:02). Herein, that the CTL “targets” cells refers to CTL recognition of cells that present on their cell surface a complex of HLA and a peptide of the present invention and demonstration of a cytotoxic activity against the cells. Further, “specifically target” refers to that the CTLs demonstrate a cytotoxic activity against those cells, but do not show a damaging activity to other cells. The expression “recognize cells” used in the context of CTLs refers to binding to a complex of HLA and a peptide of the present invention presented on cell surface via its TCR, and demonstrating a specific cytotoxic activity against the cell. Therefore, the CTLs of the present invention are preferably CTLs that can bind via TCR to a complex formed between a peptide of the present invention and HLA-A02 (more preferably HLA-A*02:01 or HLA-A*02:06) or HLA-A24 (more preferably HLA-A*24:02) presented on cell surface.

Furthermore, the CTLs of the present invention are preferably CTLs induced by a method comprising a step described in (a) or (b) below:
(a) contacting in vitro CD8-positive T cells with APCs or exosomes that present on their surface a complex of a peptide of the present invention and HLA-A02 (more preferably HLA-A*02:01 or HLA-A*02:06) or HLA-A24 (more preferably HLA-A*24:02); or
(b) introducing into CD8-positive T cells a polynucleotide encoding each subunit of a TCR capable of binding to a peptide of the present invention presented on cell surface by HLA-A02 (more preferably HLA-A*02:01 or HLA-A*02:06) or HLA-A24 (more preferably HLA-A*24:02).

### VIII. T Cell Receptors (TCRs)

The present invention also provides compositions comprising a polynucleotide encoding each subunit of a TCR capable of binding to a peptide of the present invention presented on cell surface by an HLA antigen, and methods of using the same. The polynucleotide confers CD8-positive T cells with specificity against coronavirus-infected cells through expression of a TCR capable of binding to a peptide of the present invention presented on cell surface by an HLA antigen. Polynucleotides encoding an alpha chain(s) and a beta chain(s) can be identified as the TCR subunit of the CTL induced by a peptide of the present invention by using known methods in the art (WO2007/032255 and Morgan et al., J Immunol, 171, 3288 (2003)). For example, PCR methods are preferred for TCR analysis. Without being limited thereto, PCR primers for analysis may be, for example, 5′-R Primer (5′-gtctaccaggcattcgcttcat-3′) (SEQ ID NO: 28) as a 5′ side primer; and 3-TRa-C Primer (5′-tcagctggaccacagccgcagcgt-3′) (SEQ ID NO: 29) specific to TCR-alpha-chain C-region, 3-TRb-C1 Primer (5′-tcagaaatcctttctcttgac-3′) (SEQ ID NO: 30) specific to TCR-beta-chain Cl-region or 3-TR-beta-C2 Primer (5′-ctagcctctggaatcctttctctt-3′) (SEQ ID NO: 31) specific to TCR-beta-chain C2-region as 3′ side primers. The TCRs formed by introducing the identified polynucleotides into CD8-positive T cells can bind with high binding affinity to the target cells that present a peptide of the present invention, and mediates efficient killing of the target cells presenting a peptide of the present invention in vivo and in vitro.

A polynucleotide encoding each TCR subunit can be incorporated into an appropriate vector, for example, retrovirus vector. These vectors are well known in the art. The polynucleotide or a vector comprising thereof in an expressible form can be introduced into a CD8-positive T cell, for example, a CD8-positive T cell derived from a patient. The present invention provides off-the-shelf compositions that allow rapid and easy production of modified T cells that have superior coronavirus-infected cell-killing properties by rapid modification of the patient's own T cells (or T cells derived from another subject).

Herein, a specific TCR is a TCR that can confer a specific cytotoxic activity against target cells by specifically recognizing a complex of a peptide of the present invention and an HLA antigen presented on the surface of the target cell when the TCR is present on the surface of a CD8-positive T cell. Specific recognition of the above-described complex can be confirmed by any known method, and preferable examples thereof include HLA multimer staining analysis using HLA molecules and peptides of the present invention and ELISPOT assay methods. Specific TCR-mediated recognition of target cell by T cell introduced with the above-described polynucleotide and signal transduction in the cell can be confirmed by carrying out an ELISPOT assay. When the above-described TCR is present on the surface of a CD8-positive T cell, whether the TCR can confer a target cell-specific cytotoxic activity against the CD8-positive T cell can also be confirmed by known methods. Preferable methods include, for example, measuring the cytotoxic activity against target cells by a chrome release assay method or such.

The present invention provides, in the context of HLA-A02, CTLs prepared by transforming CD8-positive T cells with a polynucleotide encoding each subunit of TCR that binds to, for example, a peptide having the amino acid sequence selected from among SEQ ID NOs: 1, 2, 3, 4, 5, 7, 9, 10, 11, 12, 13, 14 and 15. The present invention provides, in the context of HLA-A24, CTLs prepared by transforming CD8-positive T cells with a polynucleotide encoding each subunit of TCR that binds to, for example, a peptide having the amino acid sequence selected from among SEQ ID NOs: 3 and 11.

The transformed CTLs are capable of homing in vivo and may be propagated by a well-known in vitro culturing method (for example, Kawakami et al., J Immunol., 142, 3452-61 (1989)). The CTLs of the present invention can be used to form an immunogenic composition useful for disease treatment or prevention (prophylaxis) in a patient in need of treatment or prevention (prophylaxis) (the contents are incorporated herein for reference WO2006/031221).

The present invention relates to TCRs expressed by T cells that recognize a SARS-CoV-2-derived peptide. TCR is a protein molecule consisting of a dimer of an α chain and a β chain. Human T cells recognize peptides presented on MHC class I (also known as human leukocyte antigen, HLA) molecules through TCRs. As a result, T cell proliferation, differentiation, cytokine production, cytotoxic substance (perforin and granzymes) secretion and the like are induced.

The TCR-α gene comprises Vα, Jα and Cα genes. The TCR-β gene comprises Vβ, Dβ, Jβ and Cβ genes. The regions determining the specificity of TCR for a peptide are called complementarity determining regions (CDRs), which include CDR1, CDR2 and CDR3. Of these, CDR3 contacts a peptide directly, and therefore its amino acid sequence is very important in determining the antigen recognition specificity of TCR. In the TCR-α chain, V-J corresponds to CDR3, and in the TCR-β chain, V-D and D-J correspond to CDR3. Insertion and deletion of nucleotides generate their diversity.

Alternatively, in a certain embodiment of the present invention, a portion of TCR can also comprise one, two, or three complementarity determining regions (CDRs) of one or both of the α and β chains of the TCR. In a preferred embodiment, the portion of TCR comprises CDR3 of one or both of the α and β chains of the TCR. The amino acid sequences of the preferred CDR3 identified in the present invention are as follows:

CDR3 of a human T cell receptor α chain specified by any one of the amino acid sequences selected from the group consisting of SEQ ID NOs: 32, 34, 36 and 38; and

CDR3 of a human T cell receptor β chain specified by any one of the amino acid sequences selected from the group consisting of SEQ ID NOs: 33, 35 and 39.

In a certain embodiment of the present invention, the amino acid sequences of CDR3 of T cell receptor α and β chains can be combined, for example, as follows:

CDR3 of T cell receptor α chain CDR3 of T cell receptor β chain
SEQ ID NO: 32 SEQ ID NO: 33;
SEQ ID NO: 34 SEQ ID NO: 35;
SEQ ID NO: 36 SEQ ID NO: 37; and
SEQ ID NO: 38 SEQ ID NO: 39.

In a certain embodiment, α chain and β chain of TCR, TCR consisting of them, polynucleotides encoding them of the present invention may be isolated ones.

### IX. Pharmaceutical Compositions

The present invention further provides compositions or pharmaceutical compositions, comprising at least one active ingredient selected from below:
(a) a peptide of the present invention;
(b) a polynucleotide encoding a peptide of the present invention in an expressible form;
(c) an APC of the present invention;
(d) an exosome of the present invention; and
(e) a CTL of the present invention.

The pharmaceutical compositions of the present invention can comprise as needed a carrier(s), an excipient(s) or such commonly used in pharmaceuticals without particular limitations, in addition to the active ingredient(s) described above. An example of a carrier that can be used in a pharmaceutical composition of the present invention includes sterilized water, physiological saline, phosphate buffer, culture fluid and such. Therefore, the present invention also provides pharmaceutical compositions, comprising at least one active ingredient selected from (a) to (e) below and a pharmaceutically acceptable carrier:
(a) a peptide of the present invention;
(b) a polynucleotide encoding a peptide of the present invention in an expressible form;
(c) an APC of the present invention;
(d) an exosome of the present invention; and
(e) a CTL of the present invention.

Further, the pharmaceutical compositions of the present invention can comprise, as needed, stabilizers, suspensions, preservatives, surfactants, solubilizing agents, pH adjusters, aggregation inhibitors and such.

The SARS-CoV-2 protein expression significantly up-regulates in coronavirus-infected cells compared with coronavirus non-infected cells. Thus, a peptide of the present invention or a polynucleotide encoding the peptide can be used to any one of purposes selected from treatment, prevention (prophylaxis), and suppression of aggravation of coronavirus infectious disease, and combinations thereof Therefore, the present invention provides pharmaceutical compositions for any one of purposes selected from treatment, prevention (prophylaxis), and suppression of aggravation of coronavirus infectious disease, or plurality of purposes, the pharmaceutical composition comprising one or more types of peptides or polynucleotides of the present invention as an active ingredient. Alternatively, the peptides of the present invention can be made to be presented on the surface of exosomes or APCs for use as pharmaceutical compositions. In addition, CTLs of the present invention targeting any one of the peptides of the present invention can also be used as an active ingredient of the pharmaceutical compositions of the present invention. The pharmaceutical compositions of the present invention may comprise a therapeutically effective amount or a pharmaceutically effective amount of the above-described active ingredient.

The pharmaceutical compositions of the present invention may also be used as a vaccine. In the context of the present invention, the term “vaccine” (also called “immunogenic composition”) refers to a composition that has a function of inducing an immune response that leads to anti-infective action against coronavirus when inoculated into an animal. Thus, a pharmaceutical composition of the present invention can be used to induce an immune response that leads to anti-invective action against coronavirus. The immune response induced by a peptide, a polynucleotide, an APC, a CTL and a pharmaceutical composition of the present invention is not particularly limited as long as it is an immune response that leads to anti-infective action against coronavirus, and examples include induction of coronavirus-infected cell-specific CTLs and induction of coronavirus-infected cell-specific cytotoxic activity.

The pharmaceutical compositions of the present invention can be used for any one of treatment, prevention (prophylaxis), and suppression of aggravation of coronavirus infectious diseases, or combination thereof in human subjects or patients. The pharmaceutical compositions of the present invention can be used preferably to a subject positive for HLA-A02 or HLA-A24. Further, the pharmaceutical compositions of the present invention can be used preferably to treat and/or prevent coronavirus infectious diseases, and/or suppress aggravation in a subject having HLA-A02 or HLA-A24.

In another embodiment, the present invention provides use of an active ingredient selected from below in the manufacture of a pharmaceutical composition for either or both of treating and preventing coronavirus infectious disease:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

Alternatively, the present invention further provides an active ingredient selected from below for use in either or both of treating and preventing coronavirus infectious disease:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

Alternatively, the present invention further provides a method or process for manufacturing a pharmaceutical composition for either or both of treating and preventing coronavirus infectious disease, wherein the method or process comprises a step of formulating at least one active ingredient selected from below with a pharmaceutically or physiologically acceptable carrier:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

In another embodiment, the present invention further provides a method or process for manufacturing a pharmaceutical composition for either or both of treating and preventing coronavirus infectious disease, wherein the method or process comprises a step of mixing an active ingredient selected from below with a pharmaceutically or physiologically acceptable carrier:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

In another embodiment, the present invention further provides a method for either or both of treating and preventing coronavirus infectious disease, which comprises a step of administering to a subject at least one active ingredient selected from below:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

In the present invention, peptides having the amino acid sequence selected from among SEQ ID NOs: 1, 2, 3, 4, 5, 7, 9, 10, 11, 12, 13, 14 and 15 are identified as HLA-A02-restricted epitope peptides that can induce a potent and specific immune response. Therefore, pharmaceutical compositions of the present invention comprising at least one peptide having the amino acid sequence selected from among SEQ ID NOs: 1, 2, 3, 4, 5, 7, 9, 10, 11, 12, 13, 14 and 15 are suitable particularly for administration to a subject having HLA-A02 (for example, HLA-A*02:01) as an HLA antigen. The same applies to pharmaceutical compositions comprising a polynucleotide encoding any of these peptides (i.e., polynucleotides of the present invention), an APC or exosome that presents these peptides (i.e., APCs or exosomes of the present invention), or a CTL targeting these peptides (i.e., CTLs of the present invention). That is, pharmaceutical compositions comprising an active ingredient in association with a peptide having the amino acid sequence selected from among SEQ ID NOs: 1, 2, 3, 4, 5, 7, 9, 10, 11, 12, 13, 14 and 15 are suitable for administration to subjects having HLA-A02 (i.e., HLA-A02-positive subjects). In a more preferred embodiment, the pharmaceutical composition of the present invention is a pharmaceutical composition that comprises a peptide having the amino acid sequence of SEQ ID NO: 2, 3, 4, 7, 10, 11, 12 or 15.

Alternatively, in the present invention, peptides having the amino acid sequence selected from among SEQ ID NOs: 3, 4, 7, 10, 11, 12 and 15 are identified as HLA-A02-restricted epitope peptides that can induce a potent and specific immune response. Therefore, pharmaceutical compositions of the present invention comprising at least one peptide having the amino acid sequence selected from among SEQ ID NOs: 3, 4, 7, 10, 11, 12 and 15 are suitable particularly for administration to a subject having HLA-A02 (for example, HLA-A*02:06) as an HLA antigen. The same applies to pharmaceutical compositions comprising a polynucleotide encoding any of these peptides (i.e., polynucleotides of the present invention), an APC or exosome that presents these peptides (i.e., APCs or exosomes of the present invention), or a CTL targeting these peptides (i.e., CTLs of the present invention). That is, pharmaceutical compositions comprising an active ingredient in association with a peptide having the amino acid sequence selected from among SEQ ID NOs: 3, 4, 7, 10, 11, 12 and 15 are suitable for administration to subjects having HLA-A02 (i.e., HLA-A02-positive subjects). In a more preferred embodiment, the pharmaceutical composition of the present invention is a pharmaceutical composition that comprises a peptide having the amino acid sequence of SEQ ID NO: 3, 4, 7, 10, 11, 12 or 15.

Similarly, in the present invention, peptides having the amino acid sequence selected from among SEQ ID NOs: 3 and 11 are identified as HLA-A24-restricted epitope peptides that can induce a potent and specific immune response. Therefore, pharmaceutical compositions of the present invention comprising at least one peptide having the amino acid sequence selected from among SEQ ID NOs: 3 and 11 are suitable particularly for administration to a subject having HLA-A24 (for example, HLA-A*24:02) as an HLA antigen. The same applies to pharmaceutical compositions comprising a polynucleotide encoding any of these peptides (i.e., polynucleotides of the present invention), an APC or exosome that presents these peptides (i.e., APCs or exosomes of the present invention), or a CTL targeting these peptides (i.e., CTLs of the present invention). That is, pharmaceutical compositions comprising an active ingredient in association with a peptide having the amino acid sequence selected from among SEQ ID NOs: 3 and 11 are suitable for administration to subjects having HLA-A24 (i.e., HLA-A24-positive subjects). In a more preferred embodiment, the pharmaceutical composition of the present invention is a pharmaceutical composition that comprises peptides respectively having the amino acid sequences of SEQ ID NOs: 3 and 11.

Coronavirus infectious diseases to be treated and/or prevented by pharmaceutical compositions of the present invention are not particularly limited as long as they are coronavirus infectious diseases, and coronaviruses include SARS-CoV-2, MERS-CoV, SARS-CoV, and such.

In addition to the active ingredients described above, the pharmaceutical compositions of the present invention can comprise the other peptides that have the ability to induce CTLs against coronavirus-infected cells (for example, other CTL-inducing peptides derived from viral structural proteins or non-structural proteins of SARS-CoV-2, MERS-CoV and SARS-CoV), the other polynucleotides encoding the other peptides, the other cells that present the other peptides, or such.

The pharmaceutical compositions of the present invention may also optionally comprise the other therapeutic substances as an active ingredient, as long as they do not inhibit the anti-infective effects against coronavirus of the above-described active ingredients such as peptides of the present invention. For example, the pharmaceutical compositions of the present invention may optionally comprise anti-inflammatory compositions, analgesics, chemotherapeutics including antiviral agents, and the like. In addition to including the other therapeutic substances to a pharmaceutical composition of the present invention itself, one can also administer the pharmaceutical composition of the present invention sequentially or concurrently with one or more other pharmaceutical compositions. The dose of the pharmaceutical composition of the present invention and the other pharmaceutical compositions depend on, for example, the type of pharmaceutical composition used and the disease being treated, as well as the scheduling and routes of administration.

It should be understood that in consideration of the formulation type, the pharmaceutical composition of the present invention may include other components conventional in the art, in addition to the ingredients specifically mentioned herein.

The present invention also provides articles of manufacture or kits that comprise a pharmaceutical composition of the present invention. The articles of manufacture or kits of the present invention can include a container that houses the pharmaceutical composition of the present invention. An example of an appropriate container includes a bottle, a vial or a test tube, but is not limited thereto. The container may be formed of various materials such as glass or plastic. A label may be attached to the container, and the disease or disease state to which the pharmaceutical composition of the present invention should be used may be described in the label. The label may also indicate directions for administration and such.

The articles of manufacture or kits of the present invention may further comprise a second container that houses pharmaceutically acceptable diluents optionally, in addition to the container that houses the pharmaceutical composition of the present invention. The articles of manufacture or kits of the present invention may further comprise the other materials desirable from a commercial standpoint and the user's perspective, such as the other buffers, diluents, filters, injection needles, syringes, package inserts with instructions for use.

As needed, the pharmaceutical composition of the present invention can be provided in a pack or dispenser device that can contain one or more units of dosage forms containing active ingredients. The pack can include, for example, a metallic foil or a plastic foil such as a blister pack. Instructions for administration can be attached to the pack or dispenser device.

### (1) Pharmaceutical Compositions Comprising Peptide(s) as an Active Ingredient

The pharmaceutical composition comprising a peptide of the present invention can be formulated by conventional formulation methods as needed. The pharmaceutical compositions of the present invention may comprise as needed in addition to the peptide of the present invention, carriers, excipients and such commonly used in pharmaceuticals without particular limitations. Examples of carriers that can be used in pharmaceutical compositions of the present invention include sterilized water (for example, water for injection), physiological saline, phosphate buffer, phosphate buffered saline, Tris buffered saline, 0.3% glycine, culture fluid, and such. Further, the pharmaceutical compositions of the present invention may comprise as needed stabilizers, suspensions, preservatives, surfactants, solubilizing agents, pH adjusters, aggregation inhibitors, and such. The pharmaceutical compositions of the present invention can induce specific immunity against coronavirus-infected cells, and thus can be applied for the purpose of treatment or prevention (prophylaxis) of coronavirus infectious diseases.

For example, the pharmaceutical compositions of the present invention can be prepared by dissolving in pharmaceutically or physiologically acceptable water-soluble carriers such as sterilized water (for example, water for injection), physiological saline, phosphate buffer, phosphate buffered saline, and Tris buffered saline and adding, as needed, stabilizers, suspensions, preservatives, surfactants, solubilizing agents, pH adjusters, aggregation inhibitors and such, and then sterilizing the peptide solution. The method of sterilizing a peptide solution is not particularly limited, and is preferably carried out by filtration sterilization. Filtration sterilization can be performed using, for example, a filtration sterilization filter of 0.22 micro-m or less in pore diameter. The filtration-sterilized peptide solution can be administered to a subject, for example, as an injection, without being limited thereto. The pharmaceutical compositions of the present invention may be prepared as a freeze-dried formulation by freeze-drying the above-described peptide solution. The freeze-dried formulation can be prepared by filling the peptide solution prepared as described above into an appropriate container such as an ampule, a vial or a plastic container, followed by freeze drying and encapsulation into the container with a wash-sterilized rubber plug or such after pressure recovery. The freeze-dried formulation can be administered to a subject after it is re-dissolved in pharmaceutically or physiologically acceptable water-soluble carriers such as sterilized water (for example, water for injection), physiological saline, phosphate buffer, phosphate buffered saline, Tris buffered saline and such before administration. Preferred examples of pharmaceutical compositions of the present invention include injections of such a filtration-sterilized peptide solution, and freeze-dried formulations that result from freeze-drying the peptide solution. The present invention further encompasses kits comprising such a freeze-dried formulation and re-dissolving solution. The present invention also encompasses kits comprising a container that houses the freeze-dried formulation, which is a pharmaceutical composition of the present invention, and a container that houses a re-dissolving solution thereof.

The pharmaceutical compositions of the present invention can comprise a combination of two or more types of the peptides of the present invention. The combination of peptides can take a cocktail form of mixed peptides, or can be conjugated with each other using standard techniques. For example, peptides can be chemically linked or expressed as single fusion polypeptide sequences. By administering a peptide of the present invention, the peptide is presented on APCs by an HLA antigen at a high density, and then subsequently CTLs that react specifically to a complex formed between the presented peptide and the HLA antigen are induced. Alternatively, APCs (for example, DCs) are removed from a subject, and subsequently stimulated with peptides of the present invention to obtain APCs that present any of the peptides of the present invention on their cell surface. These APCs are re-administered to a subject to induce CTLs in the subject, and as a result, the aggressiveness towards coronavirus-infected cells can be increased. Neutralizing antibodies may lose their ability to block infection (viral immune escape) due to a mutation in their target epitope. In general, the effect of an antigen mutation is particularly significant in monoclonal antibodies, which depend on a single epitope for antigen-binding specificity. On the other hand, when the pharmaceutical composition comprises multiple CTL epitopes (cocktail), even if any of the epitopes is mutated, CTLs recognizing other epitopes are effective. Mixing epitopes derived from multiple proteins is an effective strategy for avoiding a reduction in therapeutic effect due to viral immune escape.

The pharmaceutical compositions of the present invention may also comprise an adjuvant known for effectively establishing cellular immunity. An adjuvant refers to a compound that enhances the immune response against an antigen that has immunological activity when administered together (or successively) with the antigen. Known adjuvants described in literatures, for example, Clin Microbiol Rev 1994, 7: 277-89, can be used. Examples of a suitable adjuvant include aluminum salts (aluminum phosphate, aluminum hydroxide, aluminum oxyhydroxide and such), alum, cholera toxin, Salmonella toxin, Incomplete Freund's adjuvant (IFA), Complete Freund's adjuvant (CFA), ISCOMatrix, GM-CSF and other immunostimulatory cytokines, oligodeoxynucleotide containing the CpG motif (CpG7909 and such), oil-in-water emulsions, Saponin or its derivatives (QS21 and such), lipopolysaccharide such as Lipid A or its derivatives (MPL, RC529, GLA, E6020 and such), lipopeptides, lactoferrin, flagellin, double-stranded RNA or its derivatives (poli IC and such), bacterial DNA, imidazoquinolines (Imiquimod, R848 and such), C-type lectin ligand (trehalose-6,6′-dibehenate (TDB) and such), CD1d ligand (alpha-galactosylceramide and such), squalene emulsions (MF59, AS03, AF03 and such), PLGA, and such, without being limited thereto. In a certain mode, the pharmaceutical composition of the present invention may comprise an adjuvant in an amount sufficient to stimulate an immune response. The adjuvant may be contained in another container separate from the pharmaceutical composition comprising a peptide of the present invention in the kits comprising the pharmaceutical composition of the present invention. In this case, the adjuvant and the pharmaceutical composition may be administered to a subject in succession, or mixed together immediately before administration to a subject. Such kits comprising a pharmaceutical composition comprising a peptide of the present invention and an adjuvant are also provided by the present invention. When the pharmaceutical composition of the present invention is a freeze-dried formulation, the kit can further comprise a re-dissolving solution. Further, the present invention provides kits comprising a container that houses a pharmaceutical composition of the present invention and a container that stores an adjuvant. The kit can further comprise as needed a container that stores the re-dissolving solution.

When an oil adjuvant is used as an adjuvant, the pharmaceutical composition of the present invention may be prepared as an emulsion. Emulsions can be prepared, for example, by mixing and stirring the peptide solution prepared as described above and an oil adjuvant. The peptide solution may be one that has been re-dissolved after freeze-drying. The emulsion may be either of the W/O-type emulsion and O/W-type emulsion, and the W/O-type emulsion is preferred for obtaining a high immune response-enhancing effect. IFA can be preferably used as an oil adjuvant, without being limited thereto. Preparation of an emulsion can be carried out immediately before administration to a subject, and in this case, the pharmaceutical composition of the present invention may be provided as a kit comprising the peptide solution of the present invention and an oil adjuvant. When the pharmaceutical composition of the present invention is a freeze-dried formulation, the kit can further comprise a re-dissolving solution. In general, an adjuvant to be combined with a peptide for administration is mixed in an amount sufficient for immunological stimulation. Alternatively, a peptide can be administered along with a sufficient amount of an adjuvant for immunological stimulation. In an embodiment, an effective amount of an adjuvant to enhance an immune response to a peptide in a subject can be mixed with the peptide, or administered to the subject along with the peptide.

Further, the pharmaceutical composition of the present invention may be a liposome formulation within which a peptide of the present invention is encapsulated, a granular formulation in which a peptide is bound to beads with several micrometers in diameter, or a formulation in which a lipid is bound to a peptide.

In another embodiment of the present invention, the peptide of the present invention may also be administered in the form of a pharmaceutically acceptable salt. Preferred examples of salts include salts with alkali metals (lithium, potassium, sodium and such), salts with alkaline-earth metals (calcium, magnesium and such), salts with other metals (copper, iron, zinc, manganese and such), salts with organic bases, salts with amines, salts with organic acids (acetic acid, formic acid, propionic acid, fumaric acid, maleic acid, succinic acid, tartaric acid, citric acid, malic acid, oxalic acid, benzoic acid, methanesulfonic acid and such), and salts with inorganic acids (hydrochloric acid, phosphoric acid, hydrobromic acid, sulfuric acid, nitric acid and such). Therefore, pharmaceutical compositions comprising a pharmaceutically acceptable salt of a peptide of the present invention are also encompassed by the present invention. Further, the “peptide of the present invention” also encompasses, in addition to the free peptide, pharmaceutically acceptable salts thereof.

In some embodiments, the pharmaceutical compositions of the present invention may further include a component which primes CTLs. Lipids have been identified as substances capable of priming CTLs in vivo against viral antigens. For example, palmitic acid residues can be attached to the epsilon- and alpha-amino groups of a lysine residue and then linked to a peptide of the present invention. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinyl-seryl-serine (P3CSS) can be used to prime CTLs when covalently attached to an appropriate peptide (see, e.g., Deres et al., Nature 1989, 342: 561-4).

Examples of methods for administering the peptides or pharmaceutical compositions of the present invention include oral, epidermal, subcutaneous, intramuscular, intraosseous, peritoneal, and intravenous injections, as well as systemic administration or local administration to the vicinity of the targeted sites, but are not limited thereto. A preferred administration method includes subcutaneous injection to the vicinity of lymph nodes such as the armpit or groin. The administration can be performed by single administration or boosted by multiple administrations. The peptides of the present invention can be administered to a subject in a therapeutically or pharmaceutically effective amount for treating coronavirus infectious disease or in a therapeutically or pharmaceutically effective amount for inducing immunity (more specifically CTLs) against coronavirus-infected cells. The dose of the peptides of the present invention can be appropriately adjusted according to the disease target for treatment or prevention (prophylaxis), the patient's age and weight, the method of administration and such. For each of the peptides of the present invention, the dose is usually 0.001 mg-1000 mg, for example, 0.01 mg-100 mg, for example, 0.1 mg-30 mg, for example, 0.1 mg-10 mg, for example, 0.5 mg-5 mg. The dosing interval can be once every several days to several months, and for example, the dosing can be done in a once-per-week interval. A skilled artisan can appropriately select a suitable dosage.

In a preferred embodiment, the pharmaceutical compositions of the present invention comprise a therapeutically effective amount of a peptide of the present invention and a pharmaceutically or physiologically acceptable carrier. In another embodiment, the pharmaceutical compositions of the present invention comprise a therapeutically effective amount of a peptide of the present invention, a pharmaceutically or physiologically acceptable carrier, and an adjuvant. The pharmaceutical compositions of the present invention can comprise 0.001 mg-1000 mg, preferably 0.01 mg-100 mg, more preferably 0.1 mg-30 mg, even more preferably 0.1 mg-10 mg, for example, 0.5 mg-5 mg of a peptide of the present invention. When a pharmaceutical composition of the present invention is an injection, it can comprise a peptide of the present invention at a concentration of 0.001 mg/ml-1000 mg/ml, preferably 0.01 mg/ml-100 mg/ml, more preferably 0.1 mg/ml-30 mg/ml, even more preferably 0.1 mg/ml-10 mg/ml, for example, 0.5 mg/ml-5 mg/ml. In this case, for example, 0.1 to 5 ml, preferably 0.5 ml to 2 ml of the pharmaceutical composition of the present invention can be administered to a subject by injection. On the other hand, when a pharmacological composition of the present invention comprises an adjuvant, the adjuvant can be contained in an amount that is effective in enhancing the immune response of a subject against the peptide.

Further, the present invention provides methods of any one of purposes selected from treatment of, prevention (prophylaxis) of and suppression of aggravation of coronavirus infectious disease, or multiple purposes, which comprise administering to a subject a therapeutically effective amount of a peptide of the present invention or a pharmaceutical composition of the present invention. As described above, the peptides of the present invention can be administered to a subject in a single dose of usually 0.001 mg-1000 mg, for example, 0.01 mg-100 mg, for example, 0.1 mg-30 mg, for example, 0.1 mg-10 mg, or for example, 0.5 mg-5 mg. In a preferred embodiment, the peptides of the present invention are administered to a subject together with an adjuvant. Further, the dosing interval can be once every several days to several months, preferably once every several days to every month, for example, once every week or once every two weeks. On the other hand, when an adjuvant is administered with a peptide in the method of the present invention, the adjuvant can be administered in an amount that is effective in enhancing the immune response of a subject against the peptide.

### (2) Pharmaceutical Compositions Containing Polynucleotides as the Active Ingredient

The pharmaceutical compositions of the present invention can also contain polynucleotides encoding the peptides of the present invention in an expressible form. Herein, the phrase “in an expressible form” means that the polynucleotide, when introduced into a cell, will be expressed as a peptide of the present invention. In an exemplified embodiment, the sequence of the polynucleotide of the present invention includes regulatory elements necessary for expression of the peptide of the present invention. The polynucleotide(s) of the present invention can be equipped with a sequence necessary to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859, 5,589,466, 5,804,566, 5,739,118, 5,736,524, 5,679,647; and WO98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The peptides of the present invention can also be expressed by viral or bacterial vectors. Examples of expression vectors include attenuated viral hosts, such as vaccinia or fowlpox. For example, as a vector to express the peptide of the present invention, vaccinia virus can be used. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 1991, 351: 456-60. A wide variety of other vectors useful for therapeutic administration or immunization, e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide of the present invention into a patient can be either direct, in which case the patient can be directly exposed to a vector harboring the polynucleotide of the present invention, or indirect, in which case, cells are first transformed with the vector harboring the polynucleotide of the present invention in vitro, then the cells are transplanted into the patient. These two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11(5): 155-215. Methods commonly known in the art of recombinant DNA technology which can also be used for the present invention are described in Ausubel et al., Current Protocols in Molecular Biology; John Wiley & Sons, NY, 1993; and Krieger, Gene Transfer and Expression, A Laboratory Manual, Stockton Press, NY, 1990. Administration may be performed by oral, intradermal, subcutaneous, or intravenous injection, and such. A systemic administration or a local administration to the vicinity of the targeted sites is used. The administration can be performed by single administration or boosted by multiple administrations. The polynucleotides of the present invention can be administered to a subject in a therapeutically or pharmaceutically effective dose for treating coronavirus infectious disease or in a therapeutically or pharmaceutically effective dose for inducing immunity (more specifically CTLs) against coronavirus-infected cells. The dose of a polynucleotide in a suitable carrier or the dose of a polynucleotide in cells transformed with a polynucleotide encoding a peptide of the present invention can be appropriately adjusted according to the disease to be treated, the patient's age and weight, the method of administration and such, and this may be usually 0.001 mg-1000 mg, for example, 0.01 mg-100 mg, for example, 0.1 mg-30 mg, for example, 0.1 mg-10 mg, or for example, 0.5 mg-5 mg. The dosing interval can be once every several days to several months, and for example, the dosing can be done in a once-per-week interval. A skilled artisan can appropriately select a suitable dosage.

### X. Methods of Using Peptides, Exosomes, APCs and CTLs

The peptides and polynucleotides of the present invention can be used to induce APCs and CTLs. CTLs can also be induced using the exosomes and APCs of the present invention. The peptides, polynucleotides, exosomes, and APCs can be used in combination with any other compound(s) as long as their CTL-inducing ability is not inhibited. Therefore, CTLs of the present invention can be induced using a pharmaceutical composition comprising any of the peptides, polynucleotides, APCs and exosomes of the present invention. Further, APCs of the present invention can be induced using a pharmaceutical composition comprising a peptide or polynucleotide of the present invention.

### (1) Methods of Inducing APCs

The present invention provides methods of inducing APCs having CTL-inducing ability, using a peptide(s) or polynucleotide(s) of the present invention.

The methods of the present invention comprise a step of contacting an APC with a peptide of the present invention in vitro, ex vivo, or in vivo. For example, a method of contacting APCs with the peptide ex vivo may comprise the steps below:
(a) collecting APCs from a subject; and
(b) contacting the APCs of step (a) with a peptide of the present invention.

The above-described APCs are not limited to a particular type of cell, and cells known to present a proteinaceous antigen on their cell surface to be recognized by lymphocytes, for example, DCs, Langerhans cells, macrophages, B cells, and activated T cells can be used. DCs have the most potent CTL-inducing ability among APCs, and thus it is preferable to use DCs. Any peptides of the present invention can be used by themselves or in combination with other peptides of the present invention. Further, peptides of the present invention can be used in combination with other CTL-inducing peptides (for example, other CTL-inducing peptides derived from viral structural proteins or non-structural proteins of SARS-CoV-2, MERS-CoV and SARS-CoV). In a certain mode, the methods of the present invention may further additionally comprise the step of collecting the APCs after step (b).

Meanwhile, when a peptide of the present invention is administered to a subject, APCs are contacted with the peptide in vivo, and as a result, APCs having a high CTL-inducing ability are induced in the body of the subject. Therefore, the methods of the present invention may comprise a step of administering a peptide of the present invention to a subject. Similarly, when a polynucleotide of the present invention is administered to a subject in an expressible form, a peptide of the present invention is expressed in vivo, the expressed peptide is contacted with APCs in vivo, and as a result APCs having a high CTL-inducing ability are induced in the body of the subject. Therefore, the present invention may also comprise a step of administering a polynucleotide of the present invention to a subject.

In order to induce APCs having CTL-inducing ability, the present invention may comprise a step of introducing a polynucleotide of the present invention into APCs. For example, the method may comprise the steps below:
(a) collecting APCs from a subject; and
(b) introducing a polynucleotide encoding a peptide of the present invention into the APCs of step (a).

Step (b) can be performed as described in the above “VI. Antigen-presenting cells (APCs)” section.

Thus, in one embodiment, the present invention provides a method of inducing APCs having CTL-inducing ability, which comprises the step (a) or (b) below:
(a) contacting APCs with a peptide of the present invention; or
(b) introducing a polynucleotide encoding a peptide of the present invention into APCs.

Furthermore, the present invention provides a method of preparing APCs having CTL-inducing ability, which comprises the step (a) or (b) below:
(a) contacting APCs with a peptide of the present invention; or
(b) introducing a polynucleotide encoding a peptide of the present invention into APCs.

The above-described methods can be performed in vitro, ex vivo, or in vivo, and it is preferable to perform them in vitro or ex vivo. APCs used in the above-described methods may be derived from a subject scheduled for administration of the induced APCs, or they may be derived from a different subject. In a certain mode, the methods of the present invention may further additionally comprise the step of collecting the APCs after step (b).

When APCs derived from a subject (donor) different from the subject scheduled for administration are used, the subject of administration and the donor must have the identical HLA type. In the methods of the present invention, when a peptide having the amino acid sequence selected from among SEQ ID NOs: 1, 2, 3, 4, 5, 7, 9, 10, 11, 12, 13, 14 and 15 or a modified peptide thereof is used as a peptide of the present invention, the HLA type is preferably HLA-A02 (more preferably HLA-A*02:01 or HLA-A*02:06) in both the subject of administration and the donor. Alternatively, APCs used in the above-described methods are preferably APCs that express HLA-A02 (more preferably HLA-A*02:01 or HLA-A*02:06).

When a peptide having the amino acid sequence selected from among SEQ ID NOs: 3 and 11 or a modified peptide thereof is used as a peptide of the present invention, the HLA type is preferably HLA-A24 (more preferably HLA-A*24:02) in both the subject of administration and the donor. Alternatively, APCs used in the above-described methods are preferably APCs that express HLA-A24 (more preferably HLA-A*24:02).

The APCs can be prepared using known methods from PBMCs after PBMCs are separated from blood collected from a donor by a specific gravity centrifugal method or such.

In another embodiment, the present invention also provides pharmaceutical compositions that comprise a peptide of the present invention or a polynucleotide encoding the peptide for inducing an APC(s) having CTL-inducing ability.

Alternatively, the present invention further provides use of a peptide of the present invention or a polynucleotide encoding the peptide in the manufacture of a pharmaceutical composition for inducing an APC(s) having CTL-inducing ability.

Alternatively, the present invention further provides peptides of the present invention or polynucleotides encoding the peptides for use in the induction of an APC(s) having CTL-inducing ability.

Alternatively, the present invention further provides methods or processes of manufacturing a pharmaceutical composition for inducing an APC(s), wherein the method or process comprises a step of formulating a peptide of the present invention or a polynucleotide encoding the peptide with a pharmaceutically or physiologically acceptable carrier.

In another embodiment, the present invention further provides methods or processes of manufacturing a pharmaceutical composition for inducing an APC(s) having CTL-inducing ability, wherein the method or process comprises a step of mixing a peptide of the present invention or a polynucleotide encoding the peptide with a pharmaceutically or physiologically acceptable carrier.

APCs induced by the methods of the present invention can induce CTLs specific to coronavirus protein (i.e., CTLs of the present invention).

### (2) Methods of Inducing CTLs

The present invention also provides methods of inducing CTLs using peptides, polynucleotides, exosomes or APCs of the present invention.

When a peptide(s), a polynucleotide(s), an exosome(s) or an APC(s) of the present invention is administered to a subject, CTLs are induced in the body of the subject and the strength of the immune response targeting coronavirus-infected cells is enhanced. Therefore, the methods of the present invention may comprise a step of administering a peptide(s), a polynucleotide(s), an APC(s) or an exosome(s) of the present invention to a subject.

Alternatively, CTLs can be induced by using them in vitro or ex vivo. For example, the methods of the present invention may include the following steps:
(a) collecting APCs from a subject;
(b) contacting the APCs of step (a) with a peptide of the present invention; and
(c) co-culturing the APCs of step (b) with CD8-positive T cells.

The induced CTLs may be returned to the subject afterwards.

The APCs to be co-cultured with the CD8-positive T cells in step (c) above can also be prepared by introducing into APCs a polynucleotide encoding a peptide of the present invention as described above in the “VI. Antigen-presenting cells (APCs)” section. However, the APCs to be used in the methods of the present invention are not limited thereto, and any APCs that present on their surface a complex of an HLA antigen and a peptide of the present invention can be used.

In the methods of the present invention, instead of such APCs, exosomes that present on their surface a complex of an HLA antigen and a peptide of the present invention can also be used. That is, the methods of the present invention can comprise a step of co-culturing with exosomes that present on their surface a complex of an HLA antigen and a peptide of the present invention. Such exosomes can be prepared by the above-described methods in the “V. Exosomes” section.

Further, CTLs can also be induced by introducing into a CD8-positive T cell a vector comprising a polynucleotide encoding each subunit of a TCR capable of binding to a peptide of the present invention presented by an HLA antigen on the cell surface. Such transformation can be carried out as described above in the “VIII. T cell receptors (TCRs)” section.

Accordingly, in one embodiment, the present invention provides methods of inducing CTLs, comprising a step selected from below:
(a) co-culturing CD8-positive T cells with APCs that present on their surface a complex of an HLA antigen and a peptide of present invention;
(b) co-culturing CD8-positive T cells with exosomes that present on their surface a complex of an HLA antigen and a peptide of present invention; and
(c) introducing into CD8-positive T cells, a vector comprising a polynucleotide encoding each subunit of a TCR capable of binding to a peptide of the present invention presented by an HLA antigen on a cell surface.

The above-described methods can be performed in vitro, ex vivo, or in vivo, and it is preferable to perform them in vitro or ex vivo. When performed in vitro or ex vivo, in a certain mode, the methods of the present invention may comprise the step of collecting induced CTLs after any one of the steps. APCs or exosomes and CD8-positive T cells used in the above-described methods may be derived from a subject scheduled for administration of the induced CTLs, or they may be derived from a different subject. When APCs or exosomes and CD8-positive T cells derived from a subject (donor) different from the subject scheduled for administration are used, the subject of administration and the donor must have the identical HLA type. For example, when a peptide having the amino acid sequence selected from among SEQ ID NOs: 1, 2, 3, 4, 5, 7, 9, 10, 11, 12, 13, 14 and 15 or a modified peptide thereof is used as peptides of the present invention, the HLA type in both the subject of administration and the donor is preferably HLA-A02 (more preferably HLA-A*02:01 or HLA-A*02:06). Alternatively, APCs or exosomes used in the above-described methods are preferably APCs or exosomes that present on their surface a complex of HLA-A02 (more preferably HLA-A*02:01 or HLA-A*02:06) and a peptide of the present invention (a peptide having the amino acid sequence selected from among SEQ ID NOs: 1, 2, 3, 4, 5, 7, 9, 10, 11, 12, 13, 14 and 15 or a modified peptide thereof). In this case, the induced CTLs show a specific cytotoxic activity against cells that present a complex of HLA-A02 and a peptide of the present invention (for example, coronavirus-infected HLA-A02-positive cells).

When a peptide having the amino acid sequence selected from among SEQ ID NOs: 3 and 11 or a modified peptide thereof is used as peptides of the present invention, the HLA type in both the subject of administration and the donor is preferably HLA-A24 (more preferably HLA-A*24:02). Alternatively, APCs or exosomes used in the above-described methods are preferably APCs or exosomes that present on their surface a complex of HLA-A24 (more preferably HLA-A*24:02) and a peptide of the present invention (a peptide having the amino acid sequence selected from among SEQ ID NOs: 3 and 11 or a modified peptide thereof). In this case, the induced CTLs show a specific cytotoxic activity against cells that present a complex of HLA-A24 and a peptide of the present invention (for example, coronavirus-infected HLA-A24-positive cells).

In another embodiment, the present invention also provides compositions or pharmaceutical compositions for inducing CTLs, comprising at least one active ingredient selected from below:
(a) a peptide of the present invention;
(b) a polynucleotide encoding a peptide of the present invention in an expressible form;
(c) an APC that presents on its surface a peptide of the present invention; and
(d) an exosome that presents on its surface a peptide of the present invention.

In another embodiment, the present invention also provides use of an active ingredient selected from below in the manufacture of compositions or pharmaceutical compositions for inducing CTLs:
(a) a peptide of the present invention;
(b) a polynucleotide encoding a peptide of the present invention in an expressible form;
(c) an APC that presents on its surface a peptide of the present invention; and
(d) an exosome that presents on its surface a peptide of the present invention.

Alternatively, the present invention further provides an active ingredient selected from below for use in inducing CTLs:
(a) a peptide of the present invention;
(b) a polynucleotide encoding a peptide of the present invention in an expressible form;
(c) an APC that presents on its surface a peptide of the present invention; and
(d) an exosome that presents on its surface a peptide of the present invention.

Alternatively, the present invention further provides a method or process for manufacturing a composition or pharmaceutical composition for inducing CTLs, which is a method or process that comprises a step of formulating an active ingredient selected from below with a pharmaceutically or physiologically acceptable carrier:
(a) a peptide of the present invention;
(b) a polynucleotide encoding a peptide of the present invention in an expressible form;
(c) an APC that presents on its surface a peptide of the present invention; and
(d) an exosome that presents on its surface a peptide of the present invention.

In another embodiment, the present invention further provides a method or process for manufacturing a composition or pharmaceutical composition for inducing CTLs, which is a method or process that comprises a step of mixing an active ingredient selected from below with a pharmaceutically or physiologically acceptable carrier:
(a) a peptide of the present invention;
(b) a polynucleotide encoding a peptide of the present invention in an expressible form;
(c) an APC that presents on its surface a peptide of the present invention; and
(d) an exosome that presents on its surface a peptide of the present invention.

### XI. Methods of Inducing an Immune Response

The present invention further provides methods of inducing an immune response against coronavirus infection. Coronaviruses include SARS-CoV-2, MERS-CoV, SARS-CoV and such, but are not limited thereto. It is preferable that the coronavirus-infected cells express HLA-A24 or HLA-A02.

The present invention also provides methods of inducing an immune response against coronavirus-infected cells. The peptides of the present invention are derived from structural or non-structural proteins of SARS-CoV-2, and are amino acid sequences commonly found also in SARS-CoV proteins and MERS-CoV proteins. Thus, when an immune response against coronavirus-infected cells is induced, proliferation of viruses in the coronavirus-infected cells is inhibited as a result. Accordingly, the present invention further provides methods of inhibiting proliferation of viruses in coronavirus-infected cells. The methods of the present invention are suitable, in particular, for inhibiting proliferation of coronaviruses in coronavirus-infected cells expressing HLA-A24 or HLA-A02.

The methods of the present invention may comprise a step of administering a composition comprising any of the peptides of the present invention or a polynucleotide(s) encoding the peptide(s). The methods of the present invention also contemplate administration of APCs or exosomes presenting any of the peptides of the present invention. The details can be referred to the “IX. Pharmaceutical compositions” section, particularly portions describing regarding use of the pharmaceutical compositions of the present invention as vaccines. In addition, exosomes and APCs that can be used in the methods of the present invention for inducing an immune response are described in detail in “V. Exosomes”, “VI. Antigen-presenting cells (APCs)” and in Items (1) and (2) of “X. Methods of using peptides, exosomes, APCs and CTLs” described above.

In another embodiment, the present invention provides pharmaceutical compositions or vaccines for inducing an immune response against coronavirus infection, wherein the pharmaceutical composition or vaccine comprises an active ingredient selected from below:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

Alternatively, the present invention also provides pharmaceutical compositions or vaccines for inducing an immune response against coronavirus-infected cells, wherein the pharmaceutical composition or vaccine comprises an active ingredient selected from below:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

Alternatively, the present invention further provides pharmaceutical compositions or vaccines for inhibiting proliferation of coronaviruses in coronavirus-infected cells, wherein the pharmaceutical composition or vaccine comprises an active ingredient selected from below:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

In another embodiment, the present invention provides use of an active ingredient selected from below in the manufacture of pharmaceutical compositions or vaccines for inducing an immune response against coronavirus infection:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

Alternatively, the present invention also provides use of an active ingredient selected from below in the manufacture of pharmaceutical compositions or vaccines for inducing an immune response against coronavirus-infected cells:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

Alternatively, the present invention further provides use of an active ingredient selected from below in the manufacture of pharmaceutical compositions or vaccines for inhibiting proliferation of coronaviruses in coronavirus-infected cells:
(a) a peptide of the present invention;
(b) a polynucleotide encoding a peptide of the present invention in an expressible form;
(c) an APC that presents on its surface a peptide of the present invention;
(d) an exosome that presents on its surface a peptide of the present invention; and
(e) a CTL of the present invention.

The present invention further provides methods or processes for manufacturing pharmaceutical compositions that induce an immune response against coronavirus infection, which is a method that may comprise a step of mixing or formulating a peptide of the present invention with a pharmaceutically acceptable carrier.

Alternatively, the present invention provides methods for inhibiting proliferation of coronaviruses in coronavirus-infected cells or methods of inducing an immune response against coronavirus infection, which comprises a step of administering to a subject vaccines or pharmaceutical compositions comprising an active ingredient selected from below:
(a) a peptide of the present invention;
(b) a polynucleotide encoding a peptide of the present invention in an expressible form;
(c) an APC that presents a peptide of the present invention on its surface;
(d) an exosome that presents a peptide of the present invention on its surface; and
(e) a CTL of the present invention.

In the context of the present invention, coronavirus infectious diseases can be treated by administering a peptide, a polynucleotide, an APC, an exosome and/or a CTL of the present invention. Alternatively, an immune response against coronavirus infection can be induced by administering a peptide, a polynucleotide, an APC, an exosome and/or a CTL of the present invention. Examples of such coronaviruses include SARS-CoV-2, MERS-CoV, SARS-CoV and such, but are not limited thereto. Further, an immune response against coronavirus-infected cells can be induced by administering a peptide, a polynucleotide, an APC, an exosome and/or a CTL of the present invention. Therefore, it is preferable to confirm whether the subject to be treated is infected with coronavirus or not before administering a vaccine or pharmaceutical composition comprising an active ingredient described above.

Thus, in one embodiment, the present invention provides a method of treating a coronavirus infectious disease in a patient in need of the infectious disease treatment, wherein the method comprises the steps below:
(i) measuring an expression level of a SARS-CoV-2 gene or a protein encoded thereby in a biological sample collected from a subject infected with coronavirus;
(ii) identifying a subject infected with coronavirus based on the expression level of the SARS-CoV-2 gene or the protein encoded thereby measured in (i); and
(iii) administering to the subject infected with coronavirus at least one active ingredient selected from the group consisting of (a) to (e) above.

Alternatively, the present invention further provides vaccines and pharmaceutical compositions comprising at least one active ingredient selected from the group consisting of (a) to (e) above for administration to a subject infected with coronavirus. The present invention further provides a method of identifying or selecting a subject to be treated with at least one active ingredient selected from the group consisting of (a) to (e) above, wherein the method comprises the steps below:
(i) measuring an expression level of a SARS-CoV-2 gene or a protein encoded thereby in a biological sample collected from a subject infected with coronavirus;
(ii) identifying a subject with coronavirus-infected cells expressing the SARS-CoV-2 gene or the protein encoded thereby based on the expression level of the SARS-CoV-2 gene or the protein encoded thereby measured in (i); and
(iii) identifying or selecting the subject identified in (ii) as a subject who may be treated with at least one active ingredient selected from the group consisting of (a) to (e) above.

Biological samples collected from a subject for measuring the expression level of a SARS-CoV-2 gene or a protein encoded thereby in the above-described methods are not particularly limited, and for example, tissue samples containing coronavirus-infected cells collected by biopsy or such can be preferably used. Alternatively, detecting coronavirus RNAs in throat swab or saliva is generally performed in identifying coronavirus-infected subject. Thus, in the present invention, a coronavirus gene includes genome RNA of coronavirus, or mRNAs transcribed therefrom. The expression level of a SARS-CoV-2 gene or a protein encoded thereby in a biological sample can be measured by known methods, and for example, methods that detect transcription products of the SARS-CoV-2 gene by probes or PCR methods (for example, cDNA microarray method, Northern blot method, RT-PCR method or such), methods that detect translation products of the SARS-CoV-2 gene by antibodies or such (for example, Western blot method, immunostaining method, immunochromatography method or such), and such can be used. Further, biological samples may be blood samples, and in this case, the blood level of an antibody against SARS-CoV-2 protein is measured, and the expression level of SARS-CoV-2 protein may be assessed based on the blood level. The blood level of an antibody against SARS-CoV-2 protein can be measured using known methods, and for example, enzyme immunoassay (EIA), enzyme-linked immunosorbent assay (ELISA), radioimmunoassay (RIA) and such using the SARS-CoV-2 protein or a peptide of the present invention as an antigen can be used.

Alternatively, the expression level of a SARS-CoV-2 protein in a subject may be assessed by detecting CTLs specific to a peptide of the present invention. The level of CTLs specific to a peptide of the present invention can be measured, for example, by isolating PBMCs from the blood collected from a subject and measuring their cytotoxic activity against target cells pulsed with the peptide of the present invention. Cytotoxic activity can be measured, for example, by the amount of interferon y released. Complexes of the peptide of the present invention and HLA, mentioned below, can also be used to measure CTL levels. The determination of whether the coronavirus-infected cells that the subject has express a SARS-CoV-2 protein may be made by comparison with the measurement results in the same type of biomaterial collected from a subject not infected with the coronavirus. That is, when the level of a measuring object in a biological sample collected from a coronavirus-infected subject is elevated compared to that in the same type of biomaterial collected from a subject not infected with the coronavirus (normal control level), the cells of the coronavirus-infected subject can be judged to express the SARS-CoV-2 protein.

In a preferred embodiment, it is preferable to confirm the HLA type of the subject before administering at least one active ingredient selected from the group consisting of (a) to (e) above. For example, for the subjects to be administered with an active ingredient in association with a peptide having the amino acid sequence selected from among SEQ ID NOs: 1, 2, 3, 4, 5, 7, 9, 10, 11, 12, 13, 14 and 15, it is preferable to select HLA-A02-positive subjects.

For the subjects to be administered with an active ingredient in association with a peptide having the amino acid sequence selected from among SEQ ID NOs: 3 and 11, it is preferable to select HLA-A24-positive subjects. Methods for determining HLA haplotypes, so-called HLA typing, are well known to those skilled in the art. Alternatively, the lymphocyte cytotoxicity test (LCT method), in which HLA types are determined by the reactivity of antibodies specific to each HLA with HLA antigens on lymphocytes, and the like are also known.

The present invention further provides complexes of a peptide of the present invention and HLA. The complexes of the present invention described above may be monomers or multimers. When a complex of the present invention is a multimer, the number of polymerization is not particularly limited, and it can be a multimer of any number of polymerization. Examples include a tetramer, pentamer, hexamer and such, but are not limited thereto. The multimers of the present invention also encompass dextramers (WO2002/072631) and streptamers (Knabel M et al., Nat Med. 2002 June; 8(6): 631-7.). Complexes of a peptide of the present invention and HLA can be prepared according to known methods (for example, Altman J D et al., Science. 1996, 274(5284): 94-6; WO2002/072631; WO2009/003492; Knabel M et al., Nat Med. 2002 June; 8(6): 631-7; and such). The complexes of the present invention, for example, can be used in the quantification of CTLs specific to a peptide of the present invention. For example, a blood sample is collected from a subject administered with a pharmaceutical composition of the present invention, and CD4-negative cells are prepared after separation of PBMCs and contacted with a fluorescent dye-conjugated complex of the present invention. Then, the percentage of CTLs specific to a peptide of the present invention can be measured by flow cytometry analysis. For example, immune response-inducing effects by a pharmaceutical composition of the present invention can be monitored by measuring CTLs specific to a peptide of the present invention before, during and/or after administration of the pharmaceutical composition of the present invention.

### XII. Antibodies

The present invention further provides antibodies that bind to the peptide of the present invention. Preferable antibodies bind specifically to a peptide of the present invention, but do not bind (or weakly bind) to one that is not the peptide of the present invention. The binding specificity of an antibody can be confirmed by inhibition assay. That is, if the binding between an antibody to be analyzed and a polypeptide (SEQ ID NOs: 17-26) consisting of an amino acid sequence of each protein encoded by a full-length genome sequence of SARS-CoV-2 is inhibited in the presence of a peptide of the present invention, this antibody is shown to specifically bind to the peptide of the present invention. Antibodies against peptides of the present invention can be used in assays of disease diagnosis and prognosis, as well as subject selection for administration of the pharmaceutical compositions of the present invention and monitoring of the pharmaceutical compositions of the present invention.

The present invention also provides various immunological assays for detecting and/or quantifying peptides of the present invention or fragments thereof. Such immunological assays include radioimmunoassay, immunochromatography, enzyme-linked immunosorbent assay (ELISA), enzyme-linked immunofluorescence assay (ELIFA) and such, without being limited thereto, and are performed within the scope of the various immunological assay formats well known in the art.

The antibodies of the present invention can be used in immunological imaging methods that can detect coronavirus-infected cells, and examples thereof include radioactive scintigraphic imaging using a labelled antibody of the present invention, without being limited thereto. Such assay methods are used clinically in the detection, monitoring, and prognosis of coronavirus-infected cells; and examples of such coronaviruses of coronavirus infectious diseases include SARS-CoV-2, MERS-CoV, SARS-CoV and such, without being limited thereto.

The antibodies of the present invention can be used in any arbitrary form such as monoclonal antibodies or polyclonal antibodies, and may further include anti-sera obtained by immunizing an animal such as a rabbit with a peptide of the present invention, all classes of polyclonal antibodies and monoclonal antibodies, human antibodies, as well as chimeric antibodies and humanized antibodies generated through gene recombination.

The peptide of the present invention or a fragment thereof used as an antigen for obtaining antibodies can be obtained by chemical synthesis or genetic engineering techniques based on the amino acid sequences disclosed herein.

The peptide used as an immunizing antigen may be a peptide of the present invention or a fragment of a peptide of the present invention. Further, the peptide may be bound to or conjugated with a carrier for increasing immunogenicity. Keyhole limpet hemocyanin (KLH) is well-known as a carrier. Methods for binding KLH to a peptide are also well known in the art.

Any mammal can be immunized with an antigen described above, and it is preferable to consider the compatibility with the parent cell used in cell fusion when generating a monoclonal antibody. Generally, animals of the order Rodentia, Lagomorpha or Primate can be used. Animals of the order Rodentia include, for example, mice, rats and hamsters. Animals of the order Lagomorpha include, for example, rabbits. Animals of the order Primate include, for example, Catarrhini monkeys (old world monkeys) such as cynomolgus monkey (Macaca fascicularis), rhesus monkeys, hamadryas, and chimpanzee.

Methods of immunizing animals with an antigen are known in the art. Intraperitoneal injection and subcutaneous injection of an antigen are standard methods for immunizing mammals. More specifically, an antigen is diluted and suspended in an appropriate amount of phosphate buffered saline (PBS), physiological saline, or such. As needed, an antigen suspension solution can be administered to mammals after being mixed with an appropriate amount of a standard adjuvant such as Freund's complete adjuvant and emulsified. Then, it is preferable to administer the antigen mixed with an appropriate amount of a Freund's incomplete adjuvant several times every 4 to 21 days. A suitable carrier may be used for immunization. After the above immunization, the serum can be examined by standard method with respect to increase in the quantity of the desired antibody.

Polyclonal antibodies against a peptide of the present invention can be prepared by collecting blood from mammals that have been confirmed with an increase in the serum level of the desired antibody after immunization, and separating the serum from blood by any conventional method. A polyclonal antibody may be a polyclonal antibody-containing serum, or a polyclonal antibody-containing fraction may be isolated from the serum. Immunoglobulin G or M can be prepared from fractions that recognize only a peptide of the present invention by, for example, using an affinity column conjugated with the peptide of the present invention, and then further purifying the fractions using a protein A or protein G column.

In order to prepare monoclonal antibodies, upon confirming an increase in the serum level of the desired antibody after immunization, immune cells are collected from the mammals and subjected to cell fusion. Immune cells used for cell fusion may be preferably obtained from the spleen. For the other parent cells fused with the above immune cells, for example, a mammalian myeloma cell, and more preferably a myeloma cell that has acquired a property for drug selection of fusion cells can be used.

The above immune cells can be fused with myeloma cells by known methods, for example, the method of Milstein et al. (Galfre and Milstein, Methods Enzymol 73: 3-46 (1981)).

Hybridomas obtained by cell fusion can be selected by culturing them in a standard selection medium such as the HAT medium (a medium containing hypoxanthine, aminopterin and thymidine). Cell culturing is typically continued in the HAT medium for a sufficient period of time (for example, several days to several weeks) to allow death of all other cells (non-fused cells) besides the desired hybridomas. Then, hybridoma cells producing the desired antibody can be screened and cloned by performing a standard limiting dilution.

In addition to the above methods of immunizing a non-human animal with an antigen for hybridoma preparation, human lymphocytes such as EB virus-infected lymphocytes can be immunized in vitro with a peptide, cells expressing the peptide, or lysates thereof. Then, the immunized lymphocytes can be fused with immortalized human-derived myeloma cells such as U266 to obtain hybridomas producing a desired human antibody capable of binding to the peptide (JP S63-17688).

Next, the obtained hybridoma is transplanted into the abdominal cavity of a mouse, and the ascites is extracted. The obtained monoclonal antibody can be purified by, for example, ammonium sulfate precipitation, protein A or protein G column, DEAE ion-exchange chromatography, or affinity column conjugated with the peptide of the present invention.

Alternatively, antibody-producing immune cells such as the immunized lymphocytes can be immortalized by a cancer gene and used for the preparation of monoclonal antibodies.

The monoclonal antibodies obtained as such can also be prepared by recombination using genetic engineering techniques (see, e.g., Borrebaeck and Larrick, Therapeutic Monoclonal Antibodies published in United Kingdom by MacMillan Publishers LTD (1990)). For example, an antibody-encoding DNA can be cloned from immune cells such as antibody-producing hybridoma or immunized lymphocytes and inserted into a suitable vector, and then this is introduced into host cells to prepare a recombinant antibody. The present invention also provides recombinant antibodies prepared as described above.

Further, the antibodies of the present invention may be antibody fragments or modified antibodies, as long as they bind to the peptides of the present invention. For example, the antibody fragments may be Fab, F(ab′)₂, Fv, or a single chain Fv (scFv) in which Fv fragments derived from an H chain and an L chain are linked with a suitable linker (Huston et al., Proc Natl Acad Sci USA 85: 5879-83 (1988)). More specifically, antibody fragments may be generated by treating an antibody with an enzyme such as papain or pepsin. Alternatively, a gene encoding an antibody fragment may be constructed, inserted into an expression vector, and expressed in an appropriate host cell (see, e.g., Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

Antibodies may be modified by conjugation with various molecules such as polyethyleneglycol (PEG). The present invention provides such modified antibodies. Modified antibodies can be obtained by chemically modifying the antibodies. These modification methods are conventional in the art.

Alternatively, the antibodies of the present invention can be obtained as chimeric antibodies of a non-human antibody-derived variable region and a human antibody-derived constant region, or as humanized antibodies comprising a non-human antibody-derived complementarity determining region (CDR) and a human antibody-derived framework region (FR) and constant region. Such antibodies can be prepared according to known techniques. Humanization can be carried out by substituting a human antibody sequence(s) with a corresponding non-human antibody CDR sequence(s) (see, e.g., Verhoeyen et al., Science 239: 1534-6 (1988)). Thus, such humanized antibodies are chimeric antibodies in which the substantially less than an intact human variable domain has been substituted with a corresponding sequence from a non-human species.

Intact human antibodies comprising a human variable region in addition to the human framework and constant regions can also be used. Such antibodies can be generated using various techniques known in the art. For example, in vitro methods include use of recombinant libraries of human antibody fragments presented on bacteriophages (for example, Hoogenboom & Winter, J. Mol. Biol. 227: 381 (1991)). Similarly, human antibodies can also be generated by introducing human immunoglobulin gene loci into transgenic animals, for example, mice, in which the endogenous immunoglobulin genes have been partially or completely inactivated. This approach is described in, for example, U.S. Pat. Nos. 6,150,584, 5,545,807, 5,545,806, 5,569,825, 5,625,126, 5,633,425 and 5,661,016.

Antibodies obtained as described above may be purified to homogeneity. For example, antibody separation and purification can be performed according to separation methods and purification methods used for general proteins. For example, an antibody can be separated and isolated by appropriately selecting and combining use of column chromatographies such as affinity chromatography, filter, ultrafiltration, salting-out, dialysis, SDS-polyacrylamide gel electrophoresis and isoelectric focusing electrophoresis (Antibodies: A Laboratory Manual. Ed Harlow and David Lane, Cold Spring Harbor Laboratory (1988)), but are not limited thereto. Protein A column and protein G column can be used as the affinity column. Exemplary protein A columns to be used include, for example, Hyper D, POROS and Sepharose F.F. (Pharmacia).

Besides affinity chromatography, exemplary chromatography includes, for example, ion-exchange chromatography, hydrophobic chromatography, gel filtration, reversed-phase chromatography, adsorption chromatography and such (Strategies for Protein Purification and Characterization: A Laboratory Course Manual. Ed Daniel R. Marshak et al., Cold Spring Harbor Laboratory Press (1996)). The chromatography procedures can be carried out by liquid-phase chromatography such as HPLC and FPLC.

The antigen-binding activity of an antibody of the present invention can be measured, for example, by using absorbance measurement, enzyme-linked immunosorbent assay (ELISA), enzyme immunoassay (EIA), radioimmunoassay (RIA), and/or immunofluorescence (IF). In the case of ELISA, an antibody of the present invention is immobilized onto a plate, a peptide of the present invention is applied to the plate, and then a sample containing the desired antibody, such as culture supernatant of antibody-producing cells or purified antibodies, is applied. Next, a secondary antibody that recognizes the primary antibody and is labelled with an enzyme such as alkaline phosphatase is applied and the plate is incubated. Then, after washing, an enzyme substrate such as p-nitrophenyl phosphate is applied to the plate, and the antigen-binding activity of the sample is evaluated by measuring absorbance. To assess the binding activity of an antibody, peptide fragments such as C-terminal or N-terminal fragments may be used as an antigen. BIAcore (Pharmacia) may be used to evaluate the activity of an antibody of the present invention.

It is possible to detect or measure a peptide of the present invention using the above methods, by exposing an antibody of the present invention to a sample assumed to contain the peptide of the present invention, and detecting or measuring an immune complex formed between the antibody and the peptide.

For example, an antibody of the present invention can be used to detect a peptide of the present invention present in the blood sample (for example, serum sample) of a subject. Alternatively, an antibody of the present invention present in the blood sample (for example, serum sample) of a subject can also be detected using a peptide of the present invention. The result of measuring a peptide of the present invention or an antibody of the present invention in the blood sample of a subject can be utilized to the subject selection for administration of the pharmaceutical compositions of the present invention or monitoring of the efficacy of the pharmaceutical compositions of the present invention.

### XIII. Vectors and Host Cells

The present invention provides vectors comprising a polynucleotide encoding a peptide of the present invention and host cells introduced with the vectors. A vector of the present invention may be used to keep a polynucleotide of the present invention in a host cell, to express a peptide of the present invention in a host cell, or to administer a polynucleotide of the present invention for gene therapy.

When E. coli is a host cell and a vector is amplified and produced in a large amount in E. coli (for example, JM109, DH5-alpha, HB101 or XL1-Blue), the vector needs to have a “replication origin” for amplification in E. coli and a marker gene for selection of transformed E. coli (for example, a drug resistance gene selected by a drug such as ampicillin, tetracycline, kanamycin, chloramphenicol). For example, the M13-series vectors, pUC-series vectors, pBR322, pBluescript, pCR-Script and such can be used. In addition, pGEM-T, pDIRECT and pT7 can be used for cloning as well as the above vectors. When a vector is used in the production of a peptide of the present invention, an expression vector can be used. For example, an expression vector for expression in E. coli needs to have the above features for amplification in E. coli. When E. coli such as JM109, DH5-alpha, HB101 or XL1-Blue are used as a host cell, the vector needs to have a promoter, for example, lacZ promoter (Ward et al., Nature 341: 544-6 (1989); FASEB J 6: 2422-7 (1989)), araB promoter (Better et al., Science 240: 1041-3 (1988)), T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may contain a signal sequence for peptide secretion. An exemplary signal sequence that directs the peptide to be secreted to the periplasm of the E. coli is the pelB signal sequence (Lei et al., J Bacteriol 169: 4379 (1987)). Means for introducing the vectors into the target host cells include, for example, the calcium chloride method and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vectors derived from yeast (e.g., “Pichia Expression Kit” (Invitrogen), pNV11, SP-Q01) and expression vectors derived from Bacillus subtilis (e.g., pPL608, pKTH50) can be used for producing the polypeptide of the present invention.

In order to express the vector in animal cells such as CHO, COS or NIH3T3 cells, the vector needs to carry a promoter necessary for expression in such cells, for example, the SV40 promoter (Mulligan et al., Nature 277: 108 (1979)), the MMLV-LTR promoter, the EF1-alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

### XIV. Method of Detecting a History of SARS-CoV-2 Infection

### Detection of a Cellular Response Due to SARS-CoV-2 Infection in Subjects

Significant IFN-γ production was shown in T cells stimulated with a particular SARS-CoV-2 protein-derived peptide (SEQ ID NO: 7, 10 or 12). Thus, CTL clones specific to any of those peptides were established and TCR sequence analysis was performed to identify the amino acid sequences of CDR3 of TCRs expressed by the CTL clones specific to the SARS-CoV-2 protein-derived peptide (Table 4). When TCRα or TCRβ or a pair of them comprising CDR3 of an amino acid sequence shown in Table 4 is detected in a subject, it means that a peptide-specific CTL response has been induced in the subject. Accordingly, an increase in a particular pair of genes in a T cell population stimulated with a SARS-CoV-2 protein-derived peptide may be useful as a surrogate marker for detecting a CTL response in a subject after stimulation. When such a CTL response is confirmed, it means that the subject was previously infected with SARS-CoV-2. In the context of the present invention, a “peptide-specific CTL response” is to be understood as meaning that a TCR formed by a pair of α and β subunits specifically recognizes a complex formed by a peptide of the present invention and an HLA molecule. As discussed above, the CTL-inducing ability of the peptides defined by the specific sequences of the present invention can be maintained even after amino acid modifications. Accordingly, in addition to stimulation with a specific peptide, even when a T cell is induced by a variant peptide, its antigen specificity is considered “peptide-specific” as long as its TCR specifically recognizes such a complex formed by the original peptide.

In a preferred embodiment, the present invention provides methods for detecting a T cell response due to SARS-CoV-2 infection in a subject, the method comprising the steps of:
(a) providing a sample obtained from a subject, wherein the sample comprises a T cell;
(b) detecting the presence of a T cell specific to a SARS-CoV-2 protein-derived peptide that has been induced by SARS-CoV-2 infection in the sample; and
(c) when the presence of the T cell is shown in (b) using a T cell receptor (TCR) as an index, the probability of previous SARS-CoV-2 infection is indicated.
   The probability of previous SARS-CoV-2 infection can be determined, for example, by detecting one or both of the α and β subunits comprising CDR3 which consists of an amino acid sequence shown in Table 4.

Specifically, for example, PBMCs are collected from the blood of a subject (HLA-A*02:01- or HLA-A*02:06-positive) whose previous history of SARS-CoV-2 infection is to be examined, and TCR repertoire is analyzed. On the other hand, for example, an individual not infected with SARS-CoV-2 (HLA-A*02:01- or HLA-A*02:06-positive) is used as a comparative control, repertoire analysis result is similarly obtained for its PBMCs, and the two are compared; when a greater number of T cells specific to SARS-CoV-2 protein-derived peptides are detected than in the comparative control, the subject is indicated to have a previous history of SARS-CoV-2 infection. As the comparative control, for example, human PBMCs before SARS-CoV-2 infection of humans was reported can be used. Specifically, human PBMCs prepared from the blood collected before December 2019 can be used as the comparative control, but it is not limited thereto.

In the present invention, any biological sample obtained from a subject can be used to detect a T cell response as long as the sample comprises T cells. For example, blood or a blood-derived sample can be used as a biological sample for the present invention. In the present invention, a blood-derived sample comprises a cell population comprising T cells. Methods for obtaining a cell population comprising T cells are well known to those skilled in the art.

A T cell receptor (TCR) consists of V, D, J and C genes.

It is considered that as much as 10 raised to the power of 18 of diversity is generated in TCRs by rearrangements of the V and J genes and further by insertions and deletions of nucleotides randomly occurring between the V-D-J genes (CDR3). Thus, there are T cells expressing various TCRs in the human body.

Examining TCR diversity (how often and what TCRs are detected) in a certain T cell population is called TCR repertoire analysis.

When TCR repertoire analysis is performed, cDNAs in which an adaptor has been added to the 5′ end are synthesized from RNAs derived from a T cell population in order to amplify various TCR genes by a PCR method without bias. A next-generation sequencer (Next-Generation Sequencing: NGS) is used to determine the nucleotide sequences of a large amount of DNA fragments (sequence library) obtained using an adaptor-specific forward primer and a TCR-α- or TCR-β-specific reverse primer.

A next-generation sequencer is a device that has the ability to determine the nucleotide sequences of millions of DNA fragments in parallel. In TCR repertoire analysis, it is necessary to determine long nucleotide sequences spanning the V, D, J and C genes constituting TCRs. Thus, among next-generation sequencers, MiSeq (Illumina), which excels in long-read analysis (determination of nucleotide sequences of approximately 300 bp), is often used.

The detection frequency of each CDR3 in a T cell population constituting a sample can be easily known by comprehensively analyzing the nucleotide sequences of mRNAs encoding

TCRs contained in the sample (it practically means analyzing the nucleotide sequences of cDNAs reverse-transcribed from the nucleotide sequences of such mRNAs). Examples of the nucleotide sequences of primers useful for determining the nucleotide sequences of CDR3 are shown below:

Forward primer (adaptor sequence common to TCR-α
and TCR-ß, SEQ ID NO: 44):
5′-GTCTCGTGGGCTCGGAGATGTGTATAAGAGACAGTATCAACGCAGAG
TGGCCAT-3′
Reverse primer (for TCR-α, SEQ ID NO: 46):
5′-TCGTCGGCAGCGTCAGATGTGTATAAGAGACAGDBDHHCAGGGTCAG
GGTTCTGGATA-3′
Reverse primer (for TCR-ß, SEQ ID NO: 45):
5′-TCGTCGGCAGCGTCAGATGTGTATAAGAGACAGDVHDVTCTGATGGC
TCAAACACAGC-3′

More specifically, the present invention provides methods of detecting a T cell response, the method comprising the steps of:
(a) extracting gDNA from PBMCs derived from a subject or synthesizing cDNAs by using RNAs extracted from the PBMCs as templates;
(b) determining the frequency of each amino acid sequence in TCRs by decoding of TCRα gene sequences and TCRβ gene sequences from the gDNA or cDNAs; and
(c) when a TCR responsive to a SARS-CoV-2-derived peptide is detected in the amino acid sequences of TCRs determined in (b), the presence of SARS-CoV-2-specific T cells induced by infection is indicated.

In a certain embodiment of the present invention, the detection frequency of a TCR responsive to a SARS-CoV-2-derived peptide can be compared to that in a comparative control. For example, a TCR repertoire analysis result of PBMCs derived from individuals not infected with SARS-CoV-2 can be used as a comparative control. Accordingly, when the detection frequency of a TCR responsive to a SARS-CoV-2-derived peptide is greater than that in a comparative control, it indicates that a SARS-CoV-2-specific T cell response has been induced.

Prior to the methods of the present invention, the step of collecting peripheral blood lymphocytes (PBMCs) from the subject can be comprised. By repeating the series of analyses over time, changes in the frequency of a TCR responsive to a SARS-CoV-2-derived peptide can be traced. For example, changes in the frequency of a TCR responsive to a SARS-CoV-2-derived peptide of the present invention can be traced after inoculation with the SARS-CoV-2-derived peptide or SARS-CoV-2 infection to evaluate the effect of enhancing an immune response.

Accordingly, the methods of detecting a T cell response of the present invention allow knowing the results of inducing an immune response in a subject to whom a peptide of the present invention has been administered. More specifically, the present invention provides methods of detecting a T cell response, the method comprising administering a peptide of the present invention to a subject and detecting a T cell response in the subject after the administration. Moreover, in the present invention, a subject in whom a sufficient T cell response has not been detected can be additionally selected as a target for booster inoculation with the peptide of the present invention.

The embodiments of the present invention are exemplified below based on the above explanation; however, the present invention is not limited to these embodiments.

[1] A peptide of less than 15 amino acids having cytotoxic T cell (CTL)-inducing ability, which comprises the amino acid sequence selected from the group below:
(a) the amino acid sequence selected from the group consisting of SEQ ID NOs: 1, 2, 3, 4, 5, 7, 9, 10, 11, 12, 13, 14 and 15; and
(b) the amino acid sequence in which one, two or several amino acids are substituted, deleted, inserted and/or added to the amino acid sequence selected from the group consisting of SEQ ID NOs: 1, 2, 3, 4, 5, 7, 9, 10, 11, 12, 13, 14 and 15.

[2] The peptide of [1], which has either or both of features below to the amino acid sequence selected from the group consisting of SEQ ID NOs: 1, 2, 3, 4, 5, 7, 9, 10, 11, 12, 13, 14 and 15:
(a) the second amino acid from the N terminus is substituted with an amino acid selected from the group consisting of leucine and methionine; and
(b) the C-terminal amino acid is substituted with an amino acid selected from the group consisting of valine and leucine.

[3] The peptide of [1], which has either or both of features below to the amino acid sequence selected from the group consisting of SEQ ID NOs: 3, 4, 7, 10, 11, 12 and 15:
(a) the second amino acid from the N terminus is substituted with an amino acid selected from the group consisting of valine and glutamine; and
(b) the C-terminal amino acid is substituted with an amino acid selected from the group consisting of valine and leucine.
   [4] The peptide of [1], which has either or both of features below to the amino acid sequence selected from the group consisting of SEQ ID NOs: 3 and 11:
(a) the second amino acid from the N terminus is substituted with an amino acid selected from the group consisting of phenylalanine, tyrosine, methionine and tryptophan; and
(b) the C-terminal amino acid is substituted with an amino acid selected from the group consisting of phenylalanine, leucine, isoleucine, tryptophan and methionine.

[5] The peptide of [1], which consists of the amino acid sequence selected from the group consisting of SEQ ID NOs: 1, 2, 3, 4, 5, 7, 9, 10, 11, 12, 13, 14 and 15.

[6] A polynucleotide, which encodes the peptide of any one of [1] to [5].

[7] A composition comprising a pharmaceutically acceptable carrier and at least one active ingredient selected from the group consisting of (a) to (e) below:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[8] The composition of [7], which is a composition for inducing a CTL(s), wherein the active ingredient is at least one active ingredient selected from the group consisting of (a) to (d) below:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen; and
(d) an exosome that presents on its cell surface a complex of the peptide of any one of [1] to [5] and an HLA antigen.

[9] The composition of [7], which is a pharmaceutical composition.

[10] The composition of [9], which is a pharmaceutical composition for one or more uses selected from the group consisting of (i) treatment of coronavirus infectious disease, (ii) prevention (prophylaxis) of coronavirus infectious disease and (iii) suppression of aggravation of coronavirus infectious disease.

[11] The composition of [9], which is for inducing an immune response against coronavirus infection.

[12] The composition of [10] or [11], wherein coronavirus of the coronavirus infectious disease is selected from the group consisting of SARS-CoV-2, MERS-CoV and SARS-CoV.

[13] The composition of any one of [7] to [12], which is formulated for administration to a subject positive for HLA-A02 or HLA-A24.

[14] A method of inducing an APC(s) having CTL-inducing ability, which comprises a step selected from the group consisting of (a) and (b) below:
(a) contacting an APC(s) with the peptide of any one of [1] to [5] in vitro, ex vivo or in vivo; and
(b) introducing a polynucleotide encoding the peptide of any one of [1] to [5] into an APC(s).

[15] A method of inducing a CTL(s), which comprises a step selected from the group consisting of (a) to (c) below:
(a) co-culturing a CD8-positive T cell(s) with an APC(s) that presents on its surface a complex of an HLA antigen and the peptide of any one of [1] to [5];
(b) co-culturing a CD8-positive T cell(s) with an exosome(s) that presents on its surface a complex of an HLA antigen and the peptide of any one of [1] to [5]; and
(c) introducing into a CD8-positive T cell(s) a polynucleotide encoding each subunit of a T cell receptor (TCR) capable of binding to the peptide of any one of [1] to [5] presented by an HLA antigen on a cell surface.

[16] An APC that presents on its surface a complex of an HLA antigen and the peptide of any one of [1] to [5].

[17] The APC of [16], which is induced by the method of [14].

[18] A CTL that targets the peptide of any one of [1] to [5].

[19] The CTL of [18], which is induced by the method of [15].

[20] A method of inducing an immune response against coronavirus infection, which comprises administering to a subject at least one active ingredient selected from the group consisting of (a) to (e) below:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[21] A method of any one purpose selected from treatment of, prevention of, and suppression of aggravation of coronavirus infectious disease, or multiple purposes thereof, which comprises administering to a subject at least one active ingredient selected from the group consisting of (a) to (e) below:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[22] An antibody that binds to the peptide of any one of [1] to [5].

[23] A method of screening for a peptide having CTL-inducing ability, which comprises the steps of:
(a) generating candidate sequences consisting of an amino acid sequence in which one, two or several amino acid residues are substituted, deleted, inserted and/or added to an original amino acid sequence consisting of the amino acid sequence selected from among SEQ ID NOs: 1, 2, 3, 4, 5, 7, 9, 10, 11, 12, 13, 14 and 15;
(b) selecting from among the candidate sequences generated in (a), a candidate sequence that does not have significant homology (sequence identity) with any known human gene product;
(c) contacting an APC(s) with a peptide consisting of the candidate sequence selected in (b);
(d) contacting the APC(s) of (c) with a CD8-positive T cell(s); and
(e) selecting a peptide having an equal to or higher CTL-inducing ability than that of a peptide consisting of the original amino acid sequence.

[24] Use of at least one active ingredient selected from the group consisting of (a) to (e) below in the manufacture of a composition for inducing an immune response against coronavirus infection:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[25] Use of at least one active ingredient selected from the group consisting of (a) to (e) below in the manufacture of a pharmaceutical composition for any one purpose selected from treatment of, prevention of, and suppression of aggravation of coronavirus infectious disease, or multiple purposes thereof:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[26] Use of at least one active ingredient selected from the group consisting of (a) to (e) below for inducing an immune response against coronavirus infection:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[27] Use of at least one active ingredient selected from the group consisting of (a) to (e) below for any one purpose selected from treatment of, prevention of, and suppression of aggravation of coronavirus infectious disease, or multiple purposes thereof:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[28] A method of inducing cytotoxic activity against a coronavirus-infected cell(s), which comprises a step of administering to a subject at least one active ingredient selected from the group consisting of (a) to (e) below:
(a) one or more types of peptides of any one of [1] to [5];
(b) one or more types of polynucleotides encoding the peptide(s) of any one of [1] to [5] in an expressible form;
(c) an antigen-presenting cell (APC) that presents on its cell surface a complex of the peptide of any one of [1] to [5] and an HLA antigen;
(d) an exosome that presents on its cell surface a complex of the peptide of any one of [1] to [5] and an HLA antigen; and
(e) a CTL that targets the peptide of any one of [1] to [5].

[29] A freeze-dried formulation comprising one or more types of peptides of any one of [1] to [5].

[30] A pharmaceutical composition, which is prepared by a method that comprises dissolving one or more types of peptides of any one of [1] to [5] in a water-soluble carrier, and performing filtration sterilization.

[31 ] A filtration-sterilized aqueous solution, which is an aqueous solution that comprises one or more types of peptides of any one of [1] to [5] and a water-soluble carrier.

[32] An emulsion comprising one or more types of peptides of any one of [1] to [5], a water-soluble carrier and an oil adjuvant.

[33] A kit comprising a container that houses the pharmaceutical composition of any one of [9] to [13] and a container that houses an adjuvant.

[34] A kit comprising a container that stores a freeze-dried formulation comprising the peptide of any one of [1] to [5], a container that stores an adjuvant, and a container that stores a re-dissolving solution for the freeze-dried formulation.

[35] A kit comprising a container that houses the composition of any one of [7] to [13] and a container that houses an adjuvant.

[36] A T cell receptor α chain comprising CDR3 specified by any amino acid sequence selected from the group consisting of SEQ ID NOs: 32, 34, 36 and 38, or CDR3 functionally equivalent thereto.

[37] A T cell receptor β chain comprising CDR3 specified by any amino acid sequence selected from the group consisting of SEQ ID NOs: 33, 35, 37 and 39, or CDR3 functionally equivalent thereto.

[38] A T cell receptor consisting of a combination of any one of the T cell receptor α chain of and any one of the T cell receptor β chain of [37].

[39] The T cell receptor of [38], wherein the amino acid sequences of CDR3 of the T cell receptor α chain and β chain are any one of the following combinations: CDR3 of T cell receptor α chain CDR3 of T cell receptor β chain
SEQ ID NO: 32 SEQ ID NO: 33;
SEQ ID NO: 34 SEQ ID NO: 35;
SEQ ID NO: 36 SEQ ID NO: 37; and
SEQ ID NO: 38 SEQ ID NO: 39.

[40] A polynucleotide encoding any one of the T cell receptor α chain of [36] and any one of the T cell receptor β chain of [37].

[41] A TCR which recognizes the peptide any one of [1]-[5] presented on an APC by an HLA antigen.

[42] A method of determining SARS-CoV-2 infectious disease, the method comprising the steps of:
(a) extracting gDNA from PBMC derived from a subject, extracting cDNAs using RNAs extracted from PBMC as templates, or synthesizing cDNAs using RNAs extracted from the PBMC as templates;
(b) determining TCR repertoire by comprehensive decoding of TCRa gene sequences and TCRβ gene sequences from the gDNA or cDNAs with a next-generation sequencer (NGS); and
(c) evaluating the presence of SARS-CoV-2-specific T cell induced by the infection by profiling the TCR repertoire using a TCR responsive to a SARS-CoV-2-derived peptide as an index.

[43] The method of [42], wherein the TCR responsive to a SARS-CoV-2-derived peptide is the TCR of [41].

[44] The method of [43], wherein the TCR comprises the amino acid sequence selected from the group consisting of SEQ ID NOs: 32-39.

The present invention is explained herein in detail with reference to its specific embodiments. However, it should be understood that the above explanation is in fact an illustrative and explanatory explanation, and is intended to explain the present invention and preferred embodiments thereof. Through routine experimentation, one skilled in the art will readily recognize that various changes and modifications can be made therein without departing from the spirit and scope of the present invention. Thus, the present invention is not confined to the above explanation, but is intended to be defined by the appended claims and equivalents thereto.

Hereinbelow, the present invention is described in more detail with reference to the Examples. Nevertheless, while the following materials, method and Examples may serve to assist one of ordinary skill in making and using certain embodiments of the present invention, there are only intended to illustrate aspects of the present invention and thus in no way to limit the scope of the present invention. One of ordinary skill in the art can use methods and materials similar or equivalent to those described herein in the practice or testing of the present invention.

### Example 1

### Materials and Methods

### Cell Lines

T2 cells (HLA-A*02:01/−), human lymphoblast cell line, were purchased from ATCC. TISI cells (HLA-A*24:02/−), human lymphoblastoid cell line, were purchased from the International Histocompatibility Working Group.

Selection of Peptides Derived from SARS-CoV-2

SARS-CoV-2 protein-derived 9 mer and 10 mer peptides that would be expected to bind to HLA-A*02:01 were determined using “NetMHC4.0” binding prediction server (http://www.cbs.dtu.dk/services/NetMHC/) (Nielsen M et al., Protein Sci 2003, 12(5): 1007-1017; Andreatta M et al., Bioinformatics 2016, 32(4): 511-517). Of these, peptides commonly found in SARS-CoV Tor2 (GenBank accession number AY274119), SARS-CoV BJ01 (GenBank accession number AY278488) and SARS-CoV GZO2 (GenBank accession number AY390556), and also in MERS-CoV (GenBank accession number JX869059) were selected as candidate epitopes (Kiyotani K et al., J Hum Genet 2020, 65(7): 569-575).

### Peptide Synthesis

The peptides were synthesized by Cosmo Bio Co., Ltd. (Tokyo, Japan) according to solid synthesis method and purified by reversed-phase high-performance liquid chromatography (HPLC). The quality (purity: 90% or higher) of the peptides was guaranteed by HPLC and mass spectrometry analysis. Peptides were dissolved in dimethyl sulfoxide (final concentration: 20 mg/mL) and stored at −80° C.

### In Vitro CTL Induction

Monocyte-derived dendritic cells (DCs) were used as antigen-presenting cells to induce cytotoxic T cells (CTLs) specific to a peptide presented on a human leukocyte antigen (HLA). DCs were prepared in vitro as has already been reported in the literature (Nakahara S et al., Cancer Res 2003, 63(14): 4112-4118). Specifically, peripheral blood mononuclear cells (PBMCs) collected from healthy volunteers (HLA-A*02:01- or HLA-A*24:02-positive) were seeded onto a tissue culture dish (Corning) to allow adhesion of monocytes in PBMCs to the dish. The cells were cultured in the presence of 1000 IU/mL granulocyte-macrophage colony-stimulating factor (R&D System) and 1000 IU/mL interleukin (IL)-4 (R&D System) for seven days. AIM-V medium (Invitrogen) containing inactivated Type AB serum (MP Biomedicals) (2% ABS/AIM-V medium) was used as a medium. DCs differentiated from monocytes by cytokine and autologous CD8-positive T cells obtained by using CD8-Positive Isolation Kit (Invitrogen) were mixed at 1:20 ratio (1.5×10⁴ DC cells and 3×10⁵ CD8-positive T cells) in a 48-well plate (Corning). Peptides were further added thereto (peptide final concentration: 20 μg/ml). Volume of 2% ABS/AIM-V medium per well was 0.5 ml, and IL-7 (R&D System) and IL-21 (Cell Genix) were added thereto (final concentrations: IL-7, 10 ng/ml; IL-21, 30 ng/ml). Three days after the start of culture, DCs and peptide were again added thereto (peptide final concentration: 20 μg/ml). DCs were prepared at time of use by the same way as described above. Seven days after the start of culture, IL-2 (Novartis), IL-7 and IL-15 (Novoprotein) were added thereto (final concentrations: IL-2, 48 IU/ml; IL-7, 5 ng/ml; and IL-15, 5 ng/ml) (Wolff M et al., Nat Protoc 2014, 9(4): 950-966).

### CTL Proliferation Procedure

CTLs were proliferated using a method similar to that reported by Riddell et al. (Walter E A et al., N Engl J Med 1995, 333(16): 1038-1044; Riddell SR et al., Nat Med 1996, 2(2): 216-223). In tissue culture flasks (FALCON), CTLs were cultured in 5% ABS/AIM-V medium (culture medium volume: 25 ml/flask) along with two human B lymphoblastoid cell lines (5 x 10⁶ cells each) treated with mitomycin C and an anti-CD3 antibody (BD biosciences, final concentration: 40 ng/ml). On the day following the start of the culture, IL-2 was added to the culture (final IL-2 concentration: 120 IU/ml). On days 5, 8, and 11, the medium was replaced with 5% ABS/AIM-V medium containing 60 IU/ml IL-2 (final IL-2 concentration: 30 IU/ml) (Yoshimura S et al., PLoS One 2014, 9(1): e85267).

### Confirmation of IFN-γ Production

To confirm peptide-specific IFN-γ production of CTLs induced using the peptide, IFN-γ ELISPOT assay and IFN-γ ELISA were performed. Peptide-pulsed T2 cells or TISI cells were prepared as target cells. IFN-γ ELISPOT assay and IFN-γ ELISA were performed according to the procedure recommended by the assay kit manufacturer.

### Results

Selection of HLA-A*02:01-Binding Peptides Derived from SARS-CoV-2 Proteins

Table 2 shows the 9 mer and 10 mer peptides derived from SARS-CoV-2 proteins predicted to bind to HLA-A*02:01 by “NetMHC 4.0” in the order of high binding affinity. A total of 15 peptides were selected as candidate epitope peptides that may have the ability to bind to HLA-A*02:01.

TABLE 2
SARS COV-2 Protein-derived Peptides
Predicted to Bind to HLA-A*02:01

| | | | | Amino Acid | Binding Affinity | |
|---|---|---|---|---|---|---|
| Peptide | Protein | Position | Length | Sequence | (nM) | SEQ ID NO |
| 1 | ORF1ab | 7041 | 10 | SLFDMSKFPL | 6 | 1 |
| 2 | ORF1ab | 6077 | 10 | LMYKGLPWNV | 7 | 2 |
| 3 | ORF1ab | 6108 | 10 | FVLWAHGFEL | 8 | 3 |
| 4 | ORF1ab | 3654 | 10 | YMPASWVMRI | 12 | 4 |
| 5 | ORF1ab | 5982 | 10 | MMGFKMNYQV | 18 | 5 |
| 6 | ORF1ab | 6115 | 10 | FELTSMKYFV | 18 | 6 |
| 7 | ORF1ab | 4089 | 10 | FTYASALWEI | 21 | 7 |
| 8 | ORF1ab | 5147 | 9 | MMILSDDAV | 35 | 8 |
| 9 | N | 315 | 10 | FGMSRIGMEV | 37 | 9 |
| 10 | ORF1ab | 5245 | 10 | TLMIERFVSL | 42 | 10 |
| 11 | ORF1ab | 6160 | 10 | YVYNPFMIDV | 51 | 11 |
| 12 | ORF1ab | 3466 | 10 | NVLAWLYAAV | 72 | 12 |
| 13 | ORF1ab | 5740 | 10 | TLEPEYENSV | 79 | 13 |
| 14 | ORF1ab | 3098 | 10 | YSFLPGVYSV | 81 | 14 |
| 15 | ORF1ab | 6165 | 10 | FMIDVQQWGF | 84 | 15 |

The number in “Position” indicates the number of the first amino acid of the peptide counted from the N terminus of the protein.
Binding affinity (nM) was calculated using “NetMHC4.0”.
Induction of HLA-A*02:01-Restricted CTLs by Peptides Derived from SARS-CoV-2 Proteins

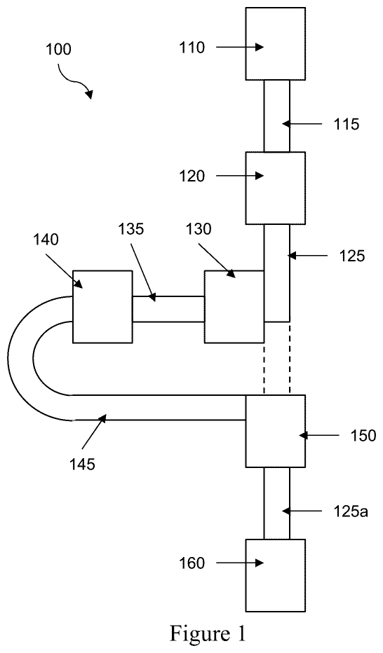

Using HLA-A*02:01-positive PBMCs, CTLs specific to peptides derived from SARS-CoV-2 proteins were induced according to the protocol described in “Materials and Methods”. Peptide-specific IFN-γ production by the cells was confirmed by an ELISPOT assay (FIG. 1). Peptide-specific IFN-γ production was observed for Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15) (FIG. 1a). On the other hand, no peptide-specific IFN-γ production was observed for the other peptides shown in Table 2. For example, peptide-specific IFN-γ production was not observed for Peptide 8 (SEQ ID NO: 8) (FIG. 1b). Although all the peptides had the possibility to bind to HLA-A*02:01, as a result, 13 peptides were identified that bind to HLA-A*02:01 and have CTL-inducing ability.

Establishment of HLA-A*02:01-Restricted CTL Lines Specific to Peptides Derived from SARS-CoV-2 Proteins

HLA-A*02:01-restricted CTL lines were established by proliferating the cells that showed IFN-γ production specific to Peptide 2 (SEQ ID NO: 2), Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15) in the HLA-A*02:01 restricted-IFN-γ ELISPOT assay. As a result of measuring IFN-γ by ELISA, IFN-γ production by CTL lines for HLA-A*02:01-expressing target cells (T2 cells) pulsed with Peptide 2 (SEQ ID NO: 2), Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15) was observed (FIG. 2). This clearly demonstrated that Peptide 2 (SEQ ID NO: 2), Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) bind to HLA-A*02:01 and have CTL-inducing ability.

Induction of HLA-A*24:02-Restricted CTLs by Peptides Derived from SARS-CoV-2 Proteins

It was verified whether Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15), which had been confirmed to have the ability to induce HLA-A*02:01-restricted CTLs, have the ability to induce HLA-A*24:02-restricted CTLs.

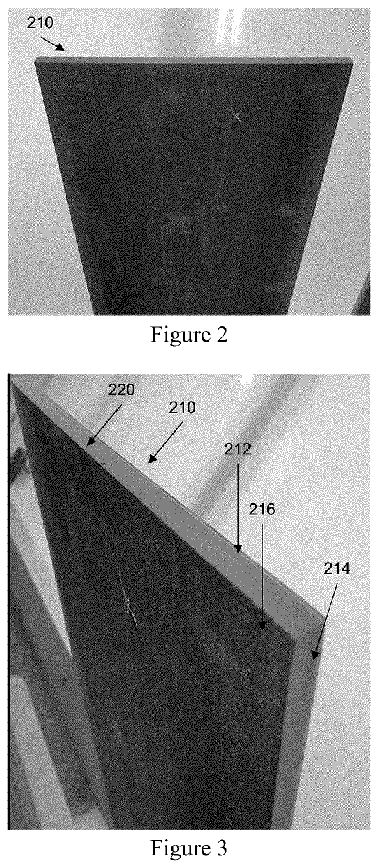

Using HLA-A*24:02-positive PBMCs, HLA-A*24:02-restricted CTLs were induced according to the protocol described in “Materials and Methods”. Peptide-specific IFN-γ production by the cells was confirmed by an ELISPOT assay (FIG. 3). Peptide-specific IFN-γ production was observed for Peptide 3 (SEQ ID NO: 3) and Peptide 11 (SEQ ID NO: 11) (FIG. 3a). On the other hand, no peptide-specific IFN-γ production was observed for Peptide 1 (SEQ ID NO: 1), Peptide 2 (SEQ ID NO: 2), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 12 (SEQ ID NO: 12), Peptide 13 (SEQ ID NO: 13), Peptide 14 (SEQ ID NO: 14) and Peptide 15 (SEQ ID NO: 15). As an example, the result of Peptide 4 (SEQ ID NO: 4) is shown (FIG. 3b). These results revealed that Peptide 3 (SEQ ID NO: 3) and Peptide 11 (SEQ ID NO: 11), which have the ability to induce HLA-A*02:01-restricted CTLs, also bind to HLA-A*24:02 and have the ability to induce HLA-A*24:02-restricted CTLs.

Establishment of HLA-A*24:02-Restricted CTL Lines Specific to Peptides Derived from SARS-CoV-2 Proteins

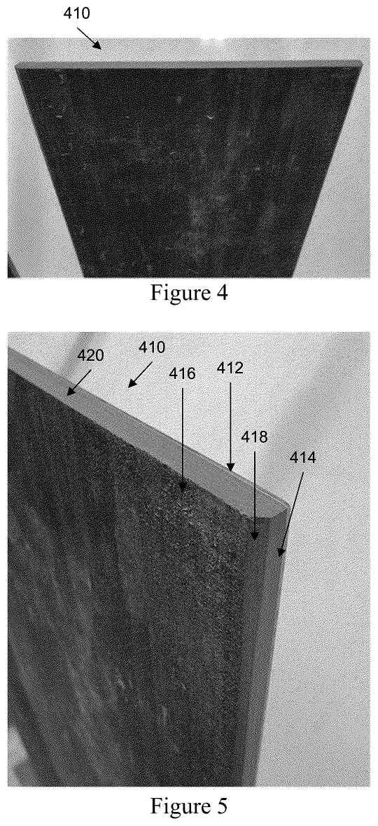

HLA-A*24:02-restricted CTL lines were established by proliferating the cells that showed IFN-γ production specific to Peptide 3 (SEQ ID NO: 3) or Peptide 11 (SEQ ID NO: 11) in the HLA-A*24:02 restricted-IFN-γ ELISPOT assay. As a result of measuring IFN-γ by ELISA, IFN-γ production by CTL lines for HLA-A*24:02-expressing target cells (TISI cells) pulsed with Peptide 3 (SEQ ID NO: 3) or Peptide 11 (SEQ ID NO: 11) was observed (FIG. 4). This clearly demonstrated that Peptide 3 (SEQ ID NO: 3) and Peptide 11 (SEQ ID NO: 11), which have the ability to induce HLA-A*02:01-restricted CTLs, also bind to HLA-A*24:02 and have the ability to induce HLA-A*24:02-restricted CTLs.

### Homology Analysis of Peptides

Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15) were confirmed to be able to induce CTLs that show peptide-specific IFN-γ production. Thus, homology analysis was performed using the BLAST algorithm (http://blast.ncbi.nlm.nih.gov/Blast.cgi) to check the homology between the amino acid sequences of Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15) and amino acid sequences derived from human proteins. As a result, the amino acid sequences of Peptide 1 (SEQ ID NO: 1), Peptide 2 (SEQ ID NO: 2), Peptide 3 (SEQ ID NO: 3), Peptide 4 (SEQ ID NO: 4), Peptide 5 (SEQ ID NO: 5), Peptide 7 (SEQ ID NO: 7), Peptide 9 (SEQ ID NO: 9), Peptide 10 (SEQ ID NO: 10), Peptide 11 (SEQ ID NO: 11), Peptide 12 (SEQ ID NO: 12), Peptide 13 (SEQ ID NO: 13), Peptide 14 (SEQ ID NO: 14) and Peptide 15 (SEQ ID NO: 15) were not found in human proteins. Accordingly, as far as the present inventors know, these peptides are derived from SARS-CoV-2 or SARS-CoV and are very unlikely to evoke an unintended immune response against human normal tissues. In conclusion, novel HLA-A*02:01-restricted epitope peptides derived from SARS-CoV-2 proteins were identified. Some of peptides were identified as HLA-A*24:02-restricted epitope peptides. It was also shown that these peptides are applicable to peptide vaccines against COVID-19.

### Example 2

### Materials and Methods

### Cell Lines

HEV0011 cells (HLA-A*02:06/−), EB virus-transformed B cell line, were purchased from Riken BioResource Research Center.

Selection of Peptides Derived from SARS-CoV-2

SARS-CoV-2 protein-derived 10 mer peptides that would be expected to bind to HLA-A*02:06 were determined using “NetMHC4.0” binding prediction server (http://www.cbs.dtu.dk/services/NetMHC/) (Nielsen M et al., Protein Sci 2003, 12(5): 1007-1017; Andreatta M et al., Bioinformatics 2016, 32(4): 511-517). Of these, peptides commonly found in SARS-CoV Tor2 (GenBank accession number AY274119), SARS-CoV BJ01 (GenBank accession number AY278488) and SARS-CoV GZO2 (GenBank accession number AY390556) were selected as candidate epitopes (Kiyotani K et al., J Hum Genet 2020, 65(7): 569-575).

### Peptide Synthesis

The peptides were synthesized by Cosmo Bio Co., Ltd. (Tokyo, Japan) according to solid synthesis method and purified by reversed-phase high-performance liquid chromatography (HPLC). The quality (purity: 90% or higher) of the peptides was guaranteed by HPLC and mass spectrometry analysis. Peptides were dissolved in dimethyl sulfoxide (final concentration: 20 mg/mL) and stored at −80° C.

### In Vitro CTL Induction

Monocyte-derived dendritic cells (DCs) were used as antigen-presenting cells to induce cytotoxic T cells (CTLs) specific to a peptide presented on a human leukocyte antigen (HLA). DCs were prepared in vitro as has already been reported in the literature (Nakahara S et al., Cancer Res 2003, 63(14): 4112-4118). Specifically, peripheral blood mononuclear cells (PBMCs) collected from healthy volunteers (HLA-A*02:06-positive) were seeded onto a tissue culture dish (Corning) to allow adhesion of monocytes in PBMCs to the dish. These cells were cultured in the presence of 1000 IU/mL granulocyte-macrophage colony-stimulating factor (R&D System) and 1000 IU/mL interleukin (IL)-4 (R&D System) for seven days. AIM-V medium (Invitrogen) containing inactivated Type AB serum (MP Biomedicals) (2% ABS/AIM-V medium) was used as a medium. DCs differentiated from monocytes by cytokine and autologous CD8-positive T cells obtained by using CD8-Positive Isolation Kit (Invitrogen) were mixed at 1:20 ratio (1.5×10⁴ DC cells and 3×10⁵ CD8-positive T cells) in a 48-well plate (Corning). Peptides were further added thereto (peptide final concentration: 20 μg/ml). Volume of 2%ABS/AIM-V medium per well was 0.5 ml, and IL-7 (R&D System) and IL-21 (Cell Genix) were added thereto (final concentrations: IL-7, 10 ng/ml; IL-21, 30 ng/ml). Three days after the start of culture, DCs and peptide were again added thereto (peptide final concentration: 20 μg/ml). DCs were prepared at time of use by the same way as described above. Seven days after the start of culture, IL-2 (Novartis), IL-7 and IL-15 (Novoprotein) were added thereto (final concentrations: IL-2, 48 IU/ml; IL-7, 5 ng/ml; and IL-15, 5 ng/ml) (Wolff M et al., Nat Protoc 2014, 9(4): 950-966).

### CTL Proliferation Procedure

CTLs were proliferated using a method similar to that reported by Riddell et al. (Walter E A et al., N Engl J Med 1995, 333(16): 1038-1044; Riddell S R et al., Nat Med 1996, 2(2): 216-223). In tissue culture flasks (FALCON), CTLs were cultured in 5% ABS/AIM-V medium (culture medium volume: 25 ml/flask) along with two human B lymphoblastoid cell lines (5×10⁶ cells each) treated with mitomycin C and an anti-CD3 antibody (BD biosciences, final concentration: 40 ng/ml). On the day following the start of the culture, IL-2 was added to the culture (final IL-2 concentration: 120 IU/ml). On days 5, 8, and 11, the medium was replaced with 5% ABS/AIM-V medium containing 60 IU/ml IL-2 (final IL-2 concentration: 30 IU/ml) (Yoshimura S et al., PLoS One 2014, 9(1): e85267).

### Confirmation of IFN-γ Production

To confirm peptide-specific IFN-γ production of CTLs induced using the peptide, IFN-γ ELISPOT assay and IFN-γ ELISA were performed. Peptide-pulsed HEV0011 cells were prepared as target cells. IFN-γ ELISPOT assay and IFN-γ ELISA were performed according to the procedure recommended by the assay kit manufacturer.

### Results

Selection of HLA-A*02:06-Binding Peptides Derived from SARS-CoV-2 Proteins

Table 3 shows the 10 mer peptides derived from SARS-CoV-2 proteins predicted to bind to HLA-A*02:06 by “NetMHC 4.0”. A total of 8 peptides were selected as candidate epitope peptides that may have the ability to bind to HLA-A*02:06.

SARS COV-2 Protein-derived Peptides
Predicted to Bind to HLA-A*02:06

| | | | | Amino Acid | Binding Affinity | |
|---|---|---|---|---|---|---|
| Peptide | Protein | Position | Length | Sequence | (nM) | SEQ ID NO |
| 2 | ORF1ab | 6077 | 10 | LMYKGLPWNV | 8 | 2 |
| 3 | ORF1ab | 6108 | 10 | FVLWAHGFEL | 13 | 3 |
| 4 | ORF1ab | 3654 | 10 | YMPASWVMRI | 103 | 4 |
| 7 | ORF1ab | 4089 | 10 | FTYASALWEI | 5 | 7 |
| 10 | ORF1ab | 5245 | 10 | TLMIERFVSL | 48 | 10 |
| 11 | ORF1ab | 6160 | 10 | YVYNPFMIDV | 15 | 11 |
| 12 | ORF1ab | 3466 | 10 | NVLAWLYAAV | 11 | 12 |
| 15 | ORF1ab | 6165 | 10 | FMIDVQQWGF | 29 | 15 |

The number in “Position” indicates the number of the first amino acid of the
peptide counted from the N terminus of the protein.
Binding affinity (nM) was calculated using “NetMHC4.0”.

Induction of HLA-A*02:06-Restricted CTLs by Peptides Derived from SARS-CoV-2 Proteins

Using HLA-A*02:06-positive PBMCs, CTLs specific to peptides derived from SARS-CoV-2 proteins were induced according to the protocol described in “Materials and Methods”. Peptide-specific IFN-γ production by the cells was confirmed by an ELISPOT assay (FIG. 5). Peptide-specific IFN-γ production was observed for Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) (FIG. 5a). On the other hand, no peptide-specific IFN-γ production was observed for the other peptides shown in Table 3. For example, peptide-specific IFN-γ production was not observed for Peptide 2 (SEQ ID NO: 2) (FIG. 5b). Although all the peptides had the possibility to bind to HLA-A*02:06, as a result, 7 peptides were identified that bind to HLA-A*02:06 and have CTL-inducing ability.

Establishment of HLA-A*02:06-Restricted CTL Lines Specific to Peptides Derived from SARS-CoV-2 Proteins

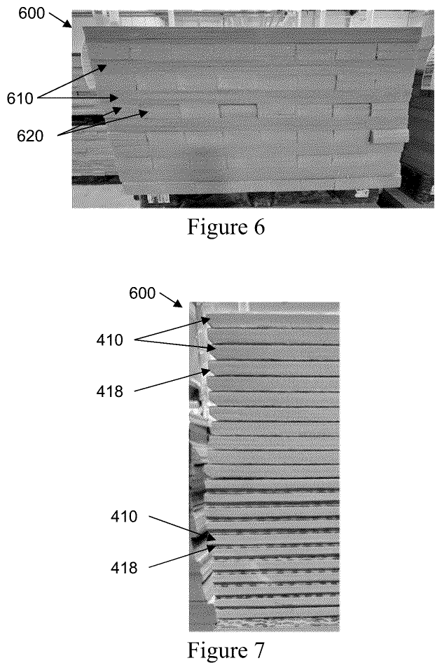

HLA-A*02:06-restricted CTL lines were established by proliferating the cells that showed IFN-γ production specific to Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15) in the HLA-A*02:06 restricted-IFN-γ ELISPOT assay. As a result of measuring IFN-γ using ELISA, IFN-γ production by CTL lines for HLA-A*02:06-expressing target cells (HEV0011 cells) pulsed with Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) or Peptide 15 (SEQ ID NO: 15) was observed (FIG. 6). This clearly demonstrated that Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) bind to HLA-A*02:06 and have CTL-inducing ability.

### Homology Analysis of Peptides

Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) were confirmed to be able to induce CTLs that show peptide-specific IFN-γ production. Thus, homology analysis was performed using the BLAST algorithm (http://blast.ncbi.nlm.nih.gov/Blast.cgi) to check the homology between the amino acid sequences of Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) and amino acid sequences derived from human proteins. As a result, the amino acid sequences of Peptide 3 (SEQ ID NO: 3), Peptide 4 (SEQ ID NO: 4), Peptide 7 (SEQ ID NO: 7), Peptide 10 (SEQ ID NO: 10), Peptide 11 (SEQ ID NO: 11), Peptide 12 (SEQ ID NO: 12) and Peptide 15 (SEQ ID NO: 15) were not found in human proteins. Accordingly, as far as the present inventors know, these peptides are derived from SARS-CoV-2 or SARS-CoV and are very unlikely to evoke an unintended immune response against human normal tissues. In conclusion, novel HLA-A*02:06-restricted epitope peptides derived from SARS-CoV-2 proteins were identified.

### Example 3

### Materials and Methods

### In Vitro CTL Induction

Monocyte-derived dendritic cells (DCs) were used as antigen-presenting cells to induce cytotoxic T cells (CTLs) specific to a peptide presented on a human leukocyte antigen (HLA). DCs were prepared in vitro as has already been reported in the literature (Nakahara S et al., Cancer Res 2003, 63(14): 4112-4118). Specifically, peripheral blood mononuclear cells (PBMCs) collected from healthy volunteers (HLA-A*02:01-positive) were seeded onto a tissue culture dish (Corning) to allow adhesion of monocytes in PBMCs to the dish. Monocytes were cultured in the presence of 1000 IU/mL granulocyte-macrophage colony-stimulating factor (R&D System) and 1000 IU/mL interleukin (IL)-4 (R&D System) for seven days. AIM-V medium (Invitrogen) containing inactivated Type AB serum (MP Biomedicals) (2% ABS/AIM-V medium) was used as a medium. DCs differentiated from monocytes by cytokine and autologous CD8-positive T cells obtained by using CD8-Positive Isolation Kit (Invitrogen) were mixed at 1:20 ratio (1.5×10⁴ DC cells and 3×10⁵ CD8-positive T cells) in a 48-well plate (Corning). Peptides were further added thereto (peptide final concentration: 20 μg/ml). Volume of 2% ABS/AIM-V medium per well was 0.5 ml, and IL-7 (R&D System) and IL-21 (Cell Genix) were added thereto (final concentrations: IL-7, 10 ng/ml; IL-21, 30 ng/ml). Three days after the start of culture, DCs and peptide were again added thereto (peptide final concentration: 20 μg/ml). DCs were prepared at time of use by the same way as described above. Seven days after the start of culture, IL-2 (Novartis), IL-7 and IL-15 (Novoprotein) were added thereto (final concentrations: IL-2, 48 IU/ml; IL-7, 5 ng/ml; and IL-15, 5 ng/ml) (Wolff M et al., Nat Protoc 2014, 9(4): 950-966).

### Establishment of CTL Clones (Limiting Dilution)

Cells after in vitro CTL induction were seeded at 1 cell/well in 96-well round-bottomed microplate (Corning). Cells were cultured with two kinds of human B-lymphoblastoid cell lines treated with Mitomycin C (each 1×10⁴ cells), anti-CD3 antibody (final concentration: 30 ng/ml), and IL-2 (final concentration: 150 IU/ml) (culture solution volume: 150 μl/well). AIM-V medium containing inactivated Type AB serum (SIGMA) (5% ABS/AIM-V medium) was used as medium. Ten days later, 50 μl of 5% ABS/AIM-V medium containing 600 IU/ml IL-2 was added to the culture (Uchida N et al., Clin Cancer Res 2004, 10(24): 8577-8586; Suda T et al., Cancer Sci 2006, 97(5): 411-419;Watanabe T et al., Cancer Sci 2005, 96(8): 498-506). On day 14 and thereafter, CTLs that showed peptide-specific IFN-γ production in ELISPOT assays were allowed to proliferate using the method described above (CTL proliferation procedure in Example 1). ELISA was performed to again verify peptide-specific IFN-γ production. The IFN-γ ELISPOT assays and IFN-γ ELISA were performed according to the procedures recommended by the assay kit manufacturers. T2 cells (HLA-A*02:01/−) (ATCC) were used as target cells.

### TCR Analysis

RNAs were extracted from CTL clones specific to a peptide derived from SARS-CoV-2 protein by RNeasy mini kit, and then cDNAs were synthesized. Sanger sequencing was conducted for determining nucleotide sequences of TCR α chain and TCR β chain. Nucleotide sequences of TCR a chain were determined after TA cloning using M13 forward primer (5′-35 TGTAAAACGACGGCCAGTG-3′, SEQ ID NO: 40) or 5′-CTGGCCGTCGTTTTAC-3′ (SEQ ID NO: 41) and M13 reverse primer (5′-CAGGAAACAGCTATGACCAT-3′, SEQ ID NO: 42) or 5′-CAGGAAACAGCTATGAC-3′ (SEQ ID NO: 43). For determining nucleotide sequences of TCR β chain, forward primer (5′-GTCTCGTGGGCTCGGAGATGTGTATAAGAGACAGTATCAACGCAGAGTGGCCAT-3′, SEQ ID NO: 44) and reverse primer (5′-TCGTCGGCAGCGTCAGATGTGTATAAGAGACAGDVHDVTCTGATGGCTCAAACACA GC-3′, SEQ ID NO: 45) were used. The nucleotide sequences of the TCRα and TCRβ genes registered in a database of TCRs, IMGT/GENE-DB (Giudicelli V et al., Nucleic Acids Res 2005, 33(Database issue):D256-261), were referred to.

### Results

### Establishment of Peptide-Specific CTL Clones

CTL clones that recognize a SARS-CoV-2 protein-derived peptide were established by a limiting dilution method. As a result of IFN-γ measurement by ELISA, the CTL clones showed IFN-γ production specific to Peptide 7 (SEQ ID NO: 7), Peptide 10 (SEQ ID NO: 10) or Peptide 12 (SEQ ID NO: 12) (FIG. 7). Thus, it was confirmed that the CTL clones recognized the SARS-CoV-2 protein-derived peptides presented on HLAs. Two clones were established as CTLs that recognize Peptide 12 (SEQ ID NO: 12).

### Identification of TCRs Expressed by Peptide-Specific CTL Clones

The CDR3 amino acid sequences of TCRs expressed by the CTL clones specific to the SARS-CoV-2 protein-derived peptide were identified by Sanger sequencing analysis (Table 4).

TABLE 4
TCRs Identified from CTL Clones Specific to
SARS-COV-2 Protein-derived Peptide

| TCR | | V Region | J Region | CDR3 Amino Acid Sequence | SEQ ID NO |
|---|---|---|---|---|---|
| Peptide 7- | alpha | TRAV21 | TRAJ48 | CAVRRTYFGNEKLTF | 32 |
| responsive TCR | beta | TRBV20-1 | TRBJ2-7 | CSARGSDSYEQYF | 33 |
| Peptide 10- | alpha | TRAV8-6 | TRAJ36 | CAITGANNLFF | 34 |
| responsive TCR | beta | TRBV20-1 | TRBJ1-5 | CSAEDRGSNSNQPQHF | 35 |
| Peptide 12- | alpha | TRAV1-2 | TRAJ20 | CAVRDPRNDYKLSF | 36 |
| responsive TCR-1 | beta | TRBV27 | TRBJ1-1 | CATQVNTEAFF | 37 |
| Peptide 12- | alpha | TRAV24 | TRAJ29 | CAFLSGNTPLVF | 38 |
| responsive TCR-2 | beta | TRBV27 | TRBJ1-6 | CASSPAAYSPLHF | 39 |

### Example 4

### Materials and Methods

### PBMCs

PBMCs derived from individuals not infected with SARS-CoV-2 (HLA-A*02:01-positive) which were collected before December 2019 were purchased from Cellular Technology Limited.

### Culture of PBMCs

The PBMCs collected from individuals not infected with SARS-CoV-2 were seeded into 48-well multiwell plates (Corning) at 5×10⁵ cells/well and then cultured for 12 days. A complete medium (a mixture of equal volume of RPMI1640 medium and AIM-V medium) containing inactivated fetal bovine serum (GIBCO) was used. Peptides were added on the start day of the culture and four days later (final concentration: 10 μg/ml). IL-2 was added 5, 7 and 10 days after the start of the culture (final concentration: 120 IU/ml). The PBMCs after the culture were used in tetramer assays.

### Production of Tetramers

QuickSwitch Quant HLA-A*02:01 Tetramer Kit-PE (MBL International Corporation) was used to produce tetramers according to the procedure recommended by the manufacturer. Fifty microliters of QuickSwitch™ Tetramer to which Exiting peptide has been bound was mixed with 1 μl of 1 mg/ml peptide solution. Furthermore, 1 μl of Peptide Exchange Factor was added and allowed to stand at room temperature for four hours or more, which resulted in obtaining tetramers in which Exiting peptide was replaced by Peptide 3 (SEQ ID NO: 3).

### Tetramer Assay

Tetramer assays were performed on the PBMCs derived from individuals not infected with SARS-CoV-2. Since T cells that recognize a peptide bind to a tetramer via TCR, the PBMCs were treated with 450 nM Dasatinib (Cayman Chemical) (37° C., 30 minutes) for maintaining TCR expression on the T cell surface (Lissina A et al., J Immunol Methods 2009, 340(1): 11-24). The PBMCs were stained with a tetramer, and then further stained with a FITC-labeled anti-CD8 antibody, an APC-labeled anti-CD3 antibody and a PE-Cy7-labeled anti-CD4 antibody (all BD Biosciences). Finally, the PBMCs were stained with 0.1 μg/ml DAPI solution (BD Biosciences) and analyzed with a flow cytometer (SH800 cell sorter, Sony). Tetramer-positive CD8-positive T cells were identified in DAPI-negative CD3-positive CD4-negative cell populations. A PE-labeled HIV tetramer (MEDICAL & BIOLOGICAL LABORATORIES CO., LTD.) was used as a negative control.

### Results

### Detection of Tetramer-Positive CD8-Positive T Cells

CD8-positive T cells recognizing Peptide 3 (SEQ ID NO: 3) were detected from PBMCs of individuals not infected with SARS-CoV-2 (FIG. 8). It was confirmed that Peptide 3 (SEQ ID NO: 3) can be recognized by some of CTLs (cross-reactive T cells) previously induced by foreign antigens. This result indicated that Peptide 3 (SEQ ID NO: 3) can be applied to a peptide vaccine against COVID-19 as antigens that stimulate T cells and elicit cellular immunity.

### Example 5

### Materials and Methods

### PBMCs

PBMCs are collected from the blood of a subject (HLA-A*02:01- or HLA-A*02:06-positive) whose previous history of SARS-CoV-2 infection is to be examined. Moreover, PBMCs derived from an individual not infected with SARS-CoV-2 (HLA-A*02:01- or HLA-A*02:06-positive) that were collected before December 2019 are purchased from Cellular Technology Limited.

### TCR Analysis

RNAs are extracted from the PBMCs using RNeasy mini kit, and then cDNAs are synthesized. Alternatively, genomic DNA (gDNA) is extracted from the PBMCs. TCR-α and TCR-β sequences are analyzed using a next-generation sequencer to perform TCR repertoire analysis. To determine the nucleotide sequences of CDR3, the following primers are used:

Forward primer (adaptor sequence common to TCR-α
and TCR-ß, SEQ ID NO: 44):
5′-GTCTCGTGGGCTCGGAGATGTGTATAAGAGACAGTATCAACGCAGA
GTGGCCAT-3′
Reverse primer (for TCR-α, SEQ ID NO: 46):
5′-TCGTCGGCAGCGTCAGATGTGTATAAGAGACAGDBDHHCAGGGTCA
GGGTTCTGGATA-3′
Reverse primer (for TCR-ß, SEQ ID NO: 45):
5′-TCGTCGGCAGCGTCAGATGTGTATAAGAGACAGDVHDVTCTGATGG
CTCAAACACAGC-3′

### Results

### TCR Repertoire Analysis of the Subject's PBMCs

The detection frequency of TCR sequences is analyzed, and when TCR comprising CDR3 of TCRa or TCRP shown in Table 4 is detected more frequently than in the comparative control (PBMCs derived from an individual not infected with SARS-CoV-2), it indicates that the subject has been previously infected with SARS-CoV-2.

### INDUSTRIAL APPLICABILITY

The present invention provides novel HLA-A24- or HLA-A02-restricted epitope peptides derived from SARS-CoV-2 proteins that induce a potent and specific immune response against coronavirus infection and thus can have applicability to a wide variety of coronavirus infectious diseases. The peptides, compositions, APCs, and CTLs of the present invention can be used as peptide vaccines against coronavirus infectious diseases, for example SARS-CoV-2, MERS-CoV, or SARS-CoV infectious diseases.

Furthermore, the sequences of TCR induced by the peptide of the present invention may be used for methods of detecting history of SARS-CoV-2 infectious diseases.

While the present invention is herein described in detail and with respect to specific embodiments thereof, it is to be understood that the foregoing description is exemplary and explanatory in nature and is intended to illustrate the present invention and its preferred embodiments. Through routine experimentation, one skilled in the art will readily recognize that various changes and modifications can be made therein without departing from the spirit and scope of the present invention, the metes and bounds of which are defined by the appended claims.

1. A peptide of less than 15 amino acids having cytotoxic T cell (CTL)-inducing ability, which comprises the amino acid sequence selected from the group below:
(a) the amino acid sequence selected from the group consisting of SEQ ID NOs: 1, 2, 3, 4, 5, 7, 9, 10, 11, 12, 13, 14 and 15; and
(b) the amino acid sequence in which one, two or several amino acids are substituted, deleted, inserted and/or added to the amino acid sequence selected from the group consisting of SEQ ID NOs: 1, 2, 3, 4, 5, 7, 9, 10, 11, 12, 13, 14 and 15.

2. The peptide of claim 1, which has either or both of features below to the amino acid sequence selected from the group consisting of SEQ ID NOs: 1, 2, 3, 4, 5, 7, 9, 10, 11, 12, 13, 14 and 15:
(a) the second amino acid from the N terminus is substituted with an amino acid selected from the group consisting of leucine and methionine; and
(b) the C-terminal amino acid is substituted with an amino acid selected from the group consisting of valine and leucine.

3. The peptide of claim 1, which has either or both of features below to the amino acid sequence selected from the group consisting of SEQ ID NOs: 3, 4, 7, 10, 11, 12 and 15:
(a) the second amino acid from the N terminus is substituted with an amino acid selected from the group consisting of valine and glutamine; and
(b) the C-terminal amino acid is substituted with an amino acid selected from the group consisting of valine and leucine.

4. The peptide of claim 1, which has either or both of features below to the amino acid sequence selected from the group consisting of SEQ ID NOs: 3 and 11:
(a) the second amino acid from the N terminus is substituted with an amino acid selected from the group consisting of phenylalanine, tyrosine, methionine and tryptophan; and
(b) the C-terminal amino acid is substituted with an amino acid selected from the group consisting of phenylalanine, leucine, isoleucine, tryptophan and methionine.

5. The peptide of claim 1, which consists of the amino acid sequence selected from the group consisting of SEQ ID NOs: 1, 2, 3, 4, 5, 7, 9, 10, 11, 12, 13, 14 and 15.

6. A polynucleotide, which encodes the peptide of any one of claims 1 to 5.

7. A composition comprising a pharmaceutically acceptable carrier and at least one active ingredient selected from the group consisting of (a) to (e) below:
(a) one or more types of peptides of any one of claims 1 to 5;
(b) one or more types of polynucleotides encoding the peptide(s) of any one of claims 1 to 5 in an expressible form;
(c) an antigen-presenting cell (APC) that presents on its cell surface a complex of the peptide of any one of claims 1 to 5 and an HLA antigen;
(d) an exosome that presents on its cell surface a complex of the peptide of any one of claims 1 to 5 and an HLA antigen; and
(e) a CTL that targets the peptide of any one of claims 1 to 5.

8. The composition of claim 7, which is a composition for inducing a CTL(s), wherein the active ingredient is at least one active ingredient selected from the group consisting of (a) to (d) below:
(a) one or more types of peptides of any one of claims 1 to 5;
(b) one or more types of polynucleotides encoding the peptide(s) of any one of claims 1 to 5 in an expressible form;
(c) an antigen-presenting cell (APC) that presents on its cell surface a complex of the peptide of any one of claims 1 to 5 and an HLA antigen; and
(d) an exosome that presents on its cell surface a complex of the peptide of any one of claims 1 to 5 and an HLA antigen.

9. The composition of claim 7, which is a pharmaceutical composition.

10. The composition of claim 9, which is a pharmaceutical composition for one or more uses selected from the group consisting of (i) treatment of coronavirus infectious disease, (ii) prevention (prophylaxis) of coronavirus infectious disease and (iii) suppression of aggravation of coronavirus infectious disease.

11. The composition of claim 9, which is for inducing an immune response against coronavirus infection.

12. The composition of claim 10 or 11, wherein coronavirus of the coronavirus infectious disease is selected from the group consisting of SARS-CoV-2, MERS-CoV and SARS-CoV.

13. The composition of any one of claims 7 to 12, which is formulated for administration to a subject positive for HLA-A24 or HLA-A02.

14. A method of inducing an APC(s) having CTL-inducing ability, which comprises a step selected from the group consisting of (a) and (b) below:
(a) contacting an APC(s) with the peptide of any one of claims 1 to 5 in vitro, ex vivo or in vivo; and
(b) introducing a polynucleotide encoding the peptide of any one of claims 1 to 5 into an APC(s).

15. A method of inducing a CTL(s), which comprises a step selected from the group consisting of (a) to (c) below:
(a) co-culturing a CD8-positive T cell(s) with an APC(s) that presents on its surface a complex of an HLA antigen and the peptide of any one of claims 1 to 5;
(b) co-culturing a CD8-positive T cell(s) with an exosome(s) that presents on its surface a complex of an HLA antigen and the peptide of any one of claims 1 to 5; and
(c) introducing into a CD8-positive T cell(s) a polynucleotide encoding each subunit of a T cell receptor (TCR) capable of binding to the peptide of any one of claims 1 to 5 presented by an HLA antigen on a cell surface.

16. An APC that presents on its surface a complex of an HLA antigen and the peptide of any one of claims 1 to 5.

17. The APC of claim 16, which is induced by the method of claim 14.

18. A CTL that targets the peptide of any one of claims 1 to 5.

19. The CTL of claim 18, which is induced by the method of claim 15.

20. A method of inducing an immune response against coronavirus infection, which comprises administering to a subject at least one active ingredient selected from the group consisting of (a) to (e) below:
(a) one or more types of peptides of any one of claims 1 to 5;
(b) one or more types of polynucleotides encoding the peptide(s) of any one of claims 1 to 5 in an expressible form;
(c) an antigen-presenting cell (APC) that presents on its cell surface a complex of the peptide of any one of claims 1 to 5 and an HLA antigen;
(d) an exosome that presents on its cell surface a complex of the peptide of any one of claims 1 to 5 and an HLA antigen; and
(e) a CTL that targets the peptide of any one of claims 1 to 5.

21. A method of treating and/or preventing coronavirus infectious disease, and/or suppressing aggravation of coronavirus infectious disease, which comprises administering to a subject at least one active ingredient selected from the group consisting of (a) to (e) below:
(a) one or more types of peptides of any one of claims 1 to 5;
(b) one or more types of polynucleotides encoding the peptide(s) of any one of claims 1 to 5 in an expressible form;
(c) an antigen-presenting cell (APC) that presents on its cell surface a complex of the peptide of any one of claims 1 to 5 and an HLA antigen;
(d) an exosome that presents on its cell surface a complex of the peptide of any one of claims 1 to 5 and an HLA antigen; and
(e) a CTL that targets the peptide of any one of claims 1 to 5.

22. An antibody that binds to the peptide of any one of claims 1 to 5.

23. A method of screening for a peptide having CTL-inducing ability, which comprises the steps of:
(a) generating candidate sequences consisting of an amino acid sequence in which one, two or several amino acid residues are substituted, deleted, inserted and/or added to an original amino acid sequence consisting of the amino acid sequence selected from among SEQ ID NOs: 1, 2, 3, 4, 5, 7, 9, 10, 11, 12, 13, 14 and 15;
(b) selecting from among the candidate sequences generated in (a), a candidate sequence that does not have significant homology (sequence identity) with any known human gene product;
(c) contacting an APC(s) with a peptide consisting of the candidate sequence selected in (b);
(d) contacting the APC(s) of (c) with a CD8-positive T cell(s); and
(e) selecting a peptide having an equal to or higher CTL-inducing ability than that of a peptide consisting of the original amino acid sequence.

24. An emulsion comprising one or more types of peptides of any one of claims 1 to 5, a water-soluble carrier and an oil adjuvant.

25. A kit comprising a container that houses the composition of any one of claims 7 to 13 and a container that houses an adjuvant.

26. A T cell receptor α chain comprising CDR3 specified by any amino acid sequence selected from the group consisting of SEQ ID NOs: 32, 34, 36 and 38, or CDR3 functionally equivalent thereto.

27. A T cell receptor β chain comprising CDR3 specified by any amino acid sequence selected from the group consisting of SEQ ID NOs: 33, 35, 37 and 39, or CDR3 functionally equivalent thereto.

28. A T cell receptor consisting of a combination of any one of the T cell receptor α chain of claim 26 and any one of the T cell receptor β chain of claim 27.

29. The T cell receptor of claim 28, wherein the amino acid sequences of CDR3 of the T cell receptor α chain and β chain are any one of the following combinations:
CDR3 of T cell receptor α chain CDR3 of T cell receptor β chain
SEQ ID NO: 32 SEQ ID NO: 33;
SEQ ID NO: 34 SEQ ID NO: 35;
SEQ ID NO: 36 SEQ ID NO: 37; and
SEQ ID NO: 38 SEQ ID NO: 39.

30. A polynucleotide encoding any one of the T cell receptor α chain of claim 26 and any one of the T cell receptor β chain of claim 27.

31. A TCR which recognizes the peptide any one of claims 1-5 presented on an APC by an HLA antigen.

32. A method of determining a history of SARS-CoV-2 infection, the method comprising the steps of:
(a) extracting gDNA from PBMC derived from a subject or synthesizing cDNAs using RNAs extracted from the PBMC as templates;
(b) determining TCR repertoire by comprehensive decoding of TCRα gene sequences and TCRβ gene sequences from the gDNA or cDNAs with a next-generation sequencer (NGS); and
(c) evaluating the presence of SARS-CoV-2-specific T cell induced by the infection by profiling the TCR repertoire using a TCR responsive to a SARS-CoV-2-derived peptide as an index.

33. The method of claim 32, wherein the TCR responsive to a SARS-CoV-2-derived peptide is the TCR of claim 31.

34. The method of claim 33, wherein the TCR comprises the amino acid sequence selected from the group consisting of SEQ ID NOs: 32-39.
